(19) United States
(12) Patent Application Publication
(10) Pub. No.: US 2024/0261388 A1
(43) Pub. Date: Aug. 8, 2024

(54) Extreme Polyvalency Induces Potent Cross-Clade Cellular and Humoral Responses in Rabbits and Non-human Primates

(71) Applicants: THE TRUSTEES OF THE UNIVERSITY OF PENNSYLVANIA, Philadelphia, PA (US); THE WISTAR INSTITUTE OF ANATOMY AND BIOLOGY, Philadelphia, PA (US)

(72) Inventors: David Weiner, Merion, PA (US); Megan Wise, Raleigh, NC (US)

(21) Appl. No.: 18/407,958

(22) Filed: Jan. 9, 2024

Related U.S. Application Data
(63) Continuation of application No. 17/093,927, filed on Nov. 10, 2020, now Pat. No. 11,883,484.
Application No. 17/093,927 is a continuation of application No. 15/705,549, filed on Sep. 15, 2017, now Pat. No. 10,828,363.
(60) Provisional application No. 62/395,803, filed on Sep. 16, 2016.

(51) Int. Cl. A61K 39/21; A61K 39/00; A61K 39/02; C07K 14/005

(52) U.S. Cl. CPC A61K 39/21; A61K 39/02; C07K 14/005; A61K 2039/53; A61K 2039/54; A61K 2039/545; C12N 2740/16134; C12N 2740/16171

(57) ABSTRACT

The present invention relates to compositions comprising two or more DNA plasmids encoding consensus and transmitted founder HIV envelope glycoproteins which expressed and induce a potent immune response.

### CROSS-REFERENCE TO RELATED APPLICATIONS

This application is a continuation of U.S. application Ser. No. 17/093,927, filed Nov. 10, 2020, which is a continuation of U.S. patent application Ser. No. 15/705,549, filed Sep. 15, 2017, which is entitled to priority under 35 U.S.0 § 119(e) to U.S. Provisional Patent Application Ser. No. 62/395,803, filed Sep. 16, 2016, the entire content of which is incorporated herein by reference.

### REFERENCE TO A “SEQUENCE LISTING” SUBMITTED AS AN XML FILE

The Sequence Listing written in the XML file: “206108-0072-02US_Sequence_Listing.xml”; created on Jan. 8, 2024, and 186,466 bytes in size, is hereby incorporated by reference.

### FIELD OF THE INVENTION

The present invention relates to treating and preventing symptoms of an HIV associated infection using a priming vaccine containing a DNA encoding the antigen, and a second vaccine for boosting the response to the first vaccine using the same or different antigen than the first vaccine.

### BACKGROUND OF THE INVENTION

A major obstacle for vaccine development is the diversity of HIV and creating an immunogen that is able to produce responses which will be broad enough to encompass the global or even regional diversity of the virus. Consensus immunogens have displayed considerable potential in driving T cell responses which exhibit cross Glade reactivity when compared to wild-type HIV immunogens (Muthumani et al., 2013, PLoS One 8:e84234; Yan et al., 2011, Vaccine 29:7173-81; Wise et al., 2015, J Virol 89:9154-66; Liao et al., 2006, Virology 353:268-82; Weaver et al., 2006, J Virol 80:6745-56; Santra et al., 2008, PNAS 105:10489-94). However, this coverage is limited to cellular responses and fails to induce a potent and broad neutralizing antibody response. Recently, it has been reported that guinea pigs vaccinated with transmitted founder gp140 Envelope proteins are able to induce low but broad neutralizing antibodies to both tier 1 and tier 2 viruses (Liao et al., 2013, J Virol 87:4185-201). This general induction of coverage may be ideal for a priming immunization, establishing a response which is able to be boosted with the addition of either chronic or consensus Envelopes.

Given the above requirement, DNA vaccination may be the optimal platform for a successful HIV vaccine. Advances in technology including codon and RNA optimization as well as electroporation, can induce anti-HIV cellular responses comparable with viral vectors (Hirao et al., 2010, Mol Ther 18:1568-76). In addition, this platform would allow for the expression of full length gp160 protein and could allow for the presentation of the native trimer to the immune system. Cryo-EM structures of Envelopes have highlighted the differences between gp120 and gp140 structures and the potential for off target effects if the proper immunogen is not provided (Lee et al., 2016, Science 351:1043-8; Mao et al., 2013, PNAS 110:12438-43; Munro and Mothes, 2015, J Virol 89:5752-5). DNA vaccination also allows for multiple difference plasmids to be delivered simultaneously, increasing the coverage of the immunization. However, while DNA vaccines against HIV are able to induce potent cellular immunity, antibody titers have remained low, and they are limited in functional antibody titers, and usually require a boost.

There is a need in the art for DNA vaccines which induce both binding and neutralizing antibodies. The present invention addresses this unmet need in the art.

### SUMMARY OF THE INVENTION

In one embodiment, the invention provides a composition comprising two or more nucleic acid molecules encoding an HIV immunogen, wherein each nucleic acid has a sequence independently selected from the group consisting of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a fragment of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a sequence that is 90% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a fragment of a sequence that is 90% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, one of SEQ ID NOs: SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61 linked to a nucleic acid encoding an IgE signal peptide, a fragment of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61 linked to a nucleic acid encoding an IgE signal peptide, a sequence that is 90% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61 linked to a nucleic acid encoding an IgE signal peptide, and a fragment of a sequence that is 90% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61 linked to a nucleic acid encoding an IgE signal peptide.

An aspect of the invention provides various immunogenic antigens of HIV selected from one or more of: Env Clade A, Env Clade B, or Env Clade C. In some embodiments the Env proteins can be selected from the following: SEQ ID NOs: 2, 4, 6, 8, 10, 12, 14, 16, 18, 20, 22, 24, 26, 28, 30, 32, 34, 36, 38, 40, 42, 44, 46, 52, 54, 56, 58, 60, or 62. In some embodiments, the vaccination of a subject can further include a HIV pol antigen, for example SEQ ID NO:48, or fragments thereof.

In one aspect, provided are various encoding nucleotide sequences that encode Env selected from one or more of: encoding sequences of Env Clade A, encoding sequences of Env Clade B, or encoding sequences of Env Clade C. The encoding sequences of Env can be selected from the following: SEQ ID NOs:1, 3, 5, 7, 9, 11, 13, 15, 17, 19, 21, 23, 25, 27, 29, 31, 33, 35, 37, 39, 41, 43, 45, 51, 53, 55, or 57; or nucleotide sequences that encode SEQ ID NOs: 2, 4, 6, 8, 10, 12, 14, 16, 18, 20, 22, 24, 26, 28, 30, 32, 34, 36, 38, 40, 42, 44, 46, 52, 54, 56, 58, 60, or 62.

In one embodiment, each nucleic acid has a sequence independently selected from the group consisting of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a fragment of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a sequence that is 95% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a fragment of a sequence that is 95% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61 linked to a nucleic acid encoding an IgE signal peptide, a fragment of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61 linked to a nucleic acid encoding an IgE signal peptide, a sequence that is 95% homologous to one of S SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61 linked to a nucleic acid encoding an IgE signal peptide, and a fragment of a sequence that is 95% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61 linked to a nucleic acid encoding an IgE signal peptide.

In one embodiment, each nucleic acid has a sequence independently selected from the group consisting of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a fragment of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a sequence that is 99% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a fragment of a sequence that is 99% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61,one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61 linked to a nucleic acid encoding an IgE signal peptide, a fragment of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61 linked to a nucleic acid encoding an IgE signal peptide, a sequence that is 99% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61 linked to a nucleic acid encoding an IgE signal peptide, and a fragment of a sequence that is 99% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61 linked to a nucleic acid encoding an IgE signal peptide.

In one embodiment, each nucleic acid has a sequence independently selected from the group consisting of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a fragment of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61 linked to a nucleic acid encoding an IgE signal peptide, and a fragment of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61 linked to a nucleic acid encoding an IgE signal peptide.

In one embodiment, the composition comprises 3 or more nucleic acid molecules. In one embodiment, the composition comprises 6 or more nucleic acid molecules. In one embodiment, the composition comprises 10 or more nucleic acid molecules. In one embodiment, the composition comprises 14 or more nucleic acid molecules.

In one embodiment, the composition comprises two or more plasmids, wherein each plasmid comprises only one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61.

In one embodiment, the composition of the invention is formulated for delivery to a subject using electroporation.

In another aspect, the invention provides a method of immunizing a subject in need thereof against HIV, the method comprising administering a first vaccine comprising one or more nucleic acid having a sequence independently selected from SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a variant thereof or a fragment thereof.

In one embodiment, the method comprises administering a second vaccine comprising one or more nucleic acid having a sequence independently selected from SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a variant thereof or a fragment thereof.

In one embodiment, the method comprises administering a third vaccine comprising one or more nucleic acid having a sequence independently selected from SEQ ID NOs: 1, 3, 5, 7, 9, 11, 13, 15, 17, 19, 21, 23, 25, 27, 29, 31, 33, 35, 37, 39, 41, 43, 45, 51, 53, 55, 57,orR 59-60, a variant thereof or a fragment thereof.

In one embodiment, the first vaccine is administered intradermally. In one embodiment, the second vaccine is administered intradermally. In one embodiment, the third vaccine is administered intramuscularly.

In one embodiment, the first vaccine is administered twice. In one embodiment, the second vaccine is administered twice. In one embodiment, the third vaccine is administered twice.

In another aspect, the invention provides a method of preventing HIV infection in an individual comprising administering a prophylactically effective amount of the composition of the invention.

### BRIEF DESCRIPTION OF THE DRAWINGS

FIG. 1, comprising FIGS. 1A and 1B, is a series of images depicting example flow plots for in vitro expression. HEK 293T cells were transfected with each plasmid. After 48 hours cells were harvested and surface expression was determined using 2G12 anti-envelope antibody followed by anti-human antibody conjugated to PE. pVax served as the negative control. All plasmid expressed in vitro as seen in FIG. 1B.

FIG. 2, comprising FIGS. 2A and 2B, is a series of images demonstrating that immunization of guinea pigs with plasmids containing primary isolate gp160 induce binding titers to consensus Glade A gp120. (FIG. 2A) Guinea pigs were immunized with 25 μg of each plasmid ID followed by electroportation. (FIG. 2B) Binding titers against consensus Glade A gp120. Individual titers are denoted in the shapes and geometric mean titers by the horizontal bar.

FIG. 3, comprising FIGS. 3A and 3B, is a series of images demonstrating that immunization of rabbits with sequential envelope plasmids induces binding titers to primary Glade A, B, and C gp120s. (FIG. 3A) Rabbits were immunized with 600 μg of each envelope construct ID followed by electroporation every three weeks for a total of 6 immunizations. (FIG. 3B) Binding titers against Glade A (92RW020), Glade B (SF162) and Glade C (ZM197) primary gp120s. Individual titers are denoted in the shapes and geometric mean titers by the horizontal bar.

FIG. 4, comprising FIGS. 4A through 4C, is a series of images demonstrating that rabbits immunized with mixed envelopes more rapidly induce humoral responses compared to separate immunization. (FIG. 4A) Rabbits were immunized with the same six Glade A envelopes as in the previous experiment but all plasmids were delivered at the same time. In order to determine if formulating the plasmids together would affect the vaccine induced responses, two separate studies were performed: one in which each plasmid was delivered at a different site and one in which all plasmids were formulated together. In both experiments, all rabbits received the same number of plasmid and amount of DNA (100 μg per plasmid for 600 μg total). All vaccinations were performed ID followed by electroporation. (FIG. 4B) Binding titers against Glade A (92RW020), Glade B (SF162) and Glade C (ZM197) primary gp120s. Individual titers are denoted in the shapes and geometric mean titers by the horizontal bar. (FIG. 4C) Neutralization titers after final immunization were determined for a set of tier 1 viruses.

FIG. 5, comprising FIGS. 5A through 5C, is a series of images demonstrating that rabbits immunized with mixed Glade B and C envelopes are able to induce strong humoral responses. (FIG. 5A) Rabbits were immunized with the either six Glade B envelopes or six Glade C envelopes. All envelope plasmids were formulated together (100 μg of each plasmid, 600 μg total) and delivered ID followed by electroporation. Binding titers against Glade A (92RW020), Glade B (SF162) and Glade C (ZM197) primary gp120s for Glade B immunized rabbits (FIG. 5B) or Glade C immunized rabbits (FIG. 5C). Individual titers are denoted in the shapes and geometric mean titers by the horizontal bar.

FIG. 6, comprising FIGS. 6A through 6C, is a series of images demonstrating that increasing the diversity of envelopes increases humoral responses. (FIG. 6A) Rabbits were immunized with two separate combinations of two Glade A, two Glade B, and two Glade C at weeks 0, 3, 6 and 9. All envelope plasmids were formulated together (100 μg of each plasmid, 600 μg total) and delivered ID followed by electroporation. (FIG. 6B) Binding titers against Glade A (92RW020), Glade B (SF162) and Glade C (ZM197) primary gp120s. Individual titers are denoted in the shapes and geometric mean titers by the horizontal bar. (FIG. 6C) Neutralization titers after final immunization were determined for a set of tier 1 viruses.

FIG. 7, comprising FIGS. 7A through 7C, is a series of images demonstrating that decreasing the percent of intra “cloud” diversity induces stronger humoral responses. (FIG. 7A) Rabbits were immunized with different combinations of Glade A, Glade B, and Glade C “clouds”. All envelope plasmids were formulated together (100 μg of each plasmid, 500 μg-600 μg total) and delivered ID followed by electroporation. (FIG. 7B) Binding titers against Glade A (92RW020), Glade B (SF162) and Glade C (ZM197) primary gp120s. Individual titers are denoted in the shapes and geometric mean titers by the horizontal bar. (FIG. 7C) Neutralization titers after final immunization were determined for a set of tier 1 viruses.

FIG. 8, comprising FIGS. 8A through 8C, is a series of images demonstrating that priming twice with the same cloud increases vaccine induced functional antibody titers. (FIG. 8A) Rabbits were immunized with different combinations of Glade A and B “clouds”. All envelope plasmids were formulated together (100 μg of each plasmid, 500 μg-600 μg total) and delivered ID followed by electroporation. (FIG. 8B) Binding titers against Glade A (92RW020), Glade B (SF162) and Glade C (ZM197) primary gp120s. Individual titers are denoted in the shapes and geometric mean titers by the horizontal bar. (FIG. 8C) Neutralization titers after final immunization were determined for a set of tier 1 viruses.

FIG. 9, comprising FIGS. 9A and 9B, is a series of images demonstrating that rhesus macaques immunized with primary envelopes from clades A and B induce robust cellular responses against consensus Glade A and B peptides. (FIG. 9A) Eight rhesus macaques were immunized with the same envelopes as in the previous study. All envelopes (1 mg per construct) were formulated together and delivered to 4-6 sites for the first 4 ID immunization. For the two IM boosting immunizations all 15 envelopes were formulated together and delivered to 1 site IM followed by electroporation. (FIG. 9B) Interferon-γ responses were determined two weeks after each vaccination and during memory period using interferon-γ ELISpots. Cells were stimulated with consensus Glade A and B peptides.

FIG. 10 is an image demonstrating that RhMs immunized with “cloud” immunizations develop broad cross-Glade binding titers. Endpoint binding titers were determined against Glade A (92RW020), Glade B (SF162) and Glade C (ZM197) primary gp120s. Individual titers are denoted in the shapes and geometric mean titers by the horizontal bar.

FIG. 11 is an image demonstrating that RhMs immunized with “cloud” immunizations develop neutralization titers which are expanded upon IM boost. Neutralization titers were determined after final ID immunization, post 1^{st} or 2^{nd} IM boost against tier 1 viruses as well as IMC for SF163P4 and SF162P3.

FIG. 12 displays graphs of binding titers. Mixing of plasmids together drives binding titers against peptides from the V3 region of gp160. Linear 15mer amino acid peptides overlapping by 11 amino acids representing the entire protein consensus sequence of HIV-1 Glade C were used to create pools for the variable regions of gp120 as well as gp41. Serum from weeks 0 and 12 were used in a binding ELISA to determine the footprint of the antibodies. There is increased induction of binding antibodies to the V3 region of gp120 in groups 3, 4, 5, and 6 and increased binding to the V1/V2 region in group2.

FIG. 13 depicts in vitro expression of primary HIV Env plasmids. 293T cells were transfected with each plasmid. Forty-eight hours later, cell lysate was harvested and western blot was performed to determine expression levels. All plasmid express Env detected by the neutralizing antibody 2G12 and the expected length.

FIG. 14, comprising FIG. 14A through FIG. 14D, depicts experimental results demonstrating the immunogenicity of each plasmid in mice. FIG. 14A depicts cellular responses post final vaccination as measured by IFN-γ spot forming units (SFU) after ex vivo stimulation of splenocytes with consensus Glade A, B or C depending on the Glade of the insert. FIG. 14B depicts humoral antibody responses as assessed by binding to consensus Glade A, B, or C gp120. FIG. 14C depicts humoral antibody responses as assessed by binding to consensus Glade A, B, or C gp140. FIG. 14D depicts binding to gp41. The dotted line represents background binding level.

FIG. 15, comprising FIG. 15A through FIG. 15D, depicts experimental results demonstrating guinea pigs immunized with mixed Envelopes induce stronger and quick humoral responses compared to separate immunization. FIG. 15A depicts the immunization scheme for guinea pig vaccination with two different groups: one where all of the plasmids were mixed and formulated together and another where each plasmid was delivered into a separate site. FIG. 15B depicts binding titers against Glade A (92RW020), Glade B (SF162) and Glade C (ZM197) primary gp120s over time. FIG. 15C depicts the avidity index of binding to 92RW020, SF162, and ZM197 at week 12. FIG. 15D depicts neutralization titers for week 12 serum were determined for a set of tier 1 viruses.

FIG. 16, comprising FIG. 16A and FIG. 16B, depicts experimental results demonstrating the expression of multiple constructs in skin. Guinea pigs were vaccinated intradermally with three constructs expressing a tagged HIV Env construct. After 24 hours, skin was biopsied and stained for expression of the tags. FIG. 16A demonstrates that expression of each individual constructs can be detected. FIG. 16B depicts an overlay of each construct demonstrating multiple constructs can be expressed form a single cell.

FIG. 17, comprising FIG. 17A through FIG. 17D, depicts experimental results demonstrating rabbits immunized with mixed Glade A, B or C Envelopes are able to induce strong humoral responses. FIG. 17A depicts rabbits were immunized with six Glade A, B or Glade C Env plasmids. All plasmids were formulated together (100 μg of each plasmid, 600 μg total) and delivered ID followed by electroporation. FIG. 17B depicts binding titers of Group 1 immunized rabbits against Glade A (92RW020), Glade B (SF162) and Glade C (ZM197) primary gp120s. FIG. 17C depicts binding titers of Group 2 immunized rabbits against Glade A (92RW020), Glade B (SF162) and Glade C (ZM197) primary gp120s. FIG. 17D depicts binding titers of Group 3 immunized rabbits against Glade A (92RW020), Glade B (SF162) and Glade C (ZM197) primary gp120s. Individual titers are denoted in the shapes, geometric mean titers by the horizontal bar and standard error by the bracket.

FIG. 18, comprising FIG. 18A through FIG. 18E depicts experimental results demonstrating clouds of Envelope plasmids increases functional humoral responses. FIG. 18A depicts the experimental design. Rabbits were immunized with 3-6 Envelope plasmids formulated together and delivered intradermally followed by EP. FIG. 18B depicts endpoint binding titers over time against 92RW020(clade A), SF162 (Glade B) and ZM197 (Glade C) for group 4 immunized rabbits. FIG. 18C depicts endpoint binding titers over time against 92RW020(clade A), SF162 (Glade B) and ZM197 (Glade C) for group 5 immunized rabbits. FIG. 18D depicts endpoint binding titers over time against 92RW020(clade A), SF162 (Glade B) and ZM197 (Glade C) for group 6 immunized rabbits. FIG. 18E depicts neutralization titers against tier 1 viruses across time for each immunization group.

FIG. 19, comprising FIG. 19A through FIG. 19E, depicts experimental results demonstrating cellular responses induced by clouds of primary HIV Env plasmids in non-human primates. FIG. 19A depicts the experimental design. Four Indian Rhesus Macaques were immunized with a combination of 14 different plasmids expressing primary HIV Envelopes following a similar immunization protocol as in rabbit group 6. FIG. 19B depicts IFN-γ ELISpot responses in peripheral blood mononuclear cells (PBMCs) after overnight stimulation with consensus Glade A and B peptides after ID immunizations. FIG. 19C depicts IFN-γ ELISpot responses in peripheral blood mononuclear cells (PBMCs) after overnight stimulation with consensus Glade A and B peptides after memory and IM boost. Cellular responses were also assessed for intracellular cytokine production of IFN-γ, IL-2 and TNF-α after stimulation with consensus Glade A, B or C peptides. FIG. 19D depicts cytokine production over the time course of immunizations for CD8 subset of CD3 T cells. FIG. 19E depicts cytokine production over the time course of immunizations for CD4 subset of CD3 T cells.

FIG. 20, comprising FIG. 20A and FIG. 20B, depicts experimental results demonstrating individual ELISpot responses over time. FIG. 20A depicts IFN-γ ELISpot responses over time for each individual NHP after ID immunizations. FIG. 20B depicts IFN-γ ELISpot responses over time for each individual NHP after memory and IM boost. NHP 4 died due to unrelated causes on week 80.

FIG. 21, comprising FIG. 21A through FIG. 21D, depicts experimental results demonstrating strong humoral binding responses induced by clouds of plasmids expressing primary HIV Envs. FIG. 21A depicts endpoint binding titers over time against 92RW020, SF162 and ZM197. FIG. 21B depicts avidity index against 92RW020, SF162 and ZM197 after the second, third, fourth ID immunization and each of the IM boost. FIG. 21C depicts binding to consensus and primary gp120/gp140 Envs as assessed by binding antibody multiplex assay (BAMA). FIG. 21D depicts antibody binding responses to multiple scaffolded (gp70) V1/V2 after final ID immunization and after each IM boosts.

FIG. 22, comprising FIG. 22A through FIG. 22D, depicts experimental results demonstrating DNA immunization alone induced functional antibody titers. In order to further understand the vaccine induced humoral response induced by the cloud DNA vaccination, both neutralization titers as well as ADCC activity were assess over the time course of immunizations. FIG. 22A depicts neutralization titers against a panel of tier 1 viruses across time. FIG. 22B depicts week 83 serum (two week post final immunization) was assessed for neutralization capacity against two infectious molecular clones: SF162P4 (tier 1) and SF162P3 (tier 2). FIG. 22C depicts antibody dependent cellular cytotoxicity (ADCC) titers were determine against targets coated with gp140 (1086c) or gp120 (WITO, JR-FL, and 92MG037.1) for serum from weeks 20 (post final ID), week 46 (post 1^{st} IM) and 83 (post 2^{nd} IM). FIG. 22D depicts a strong correlation between binding to 1086c gp140 as assessed by BAMA and ADCC titers against 1086c gp140.

FIG. 23 depicts experimental results demonstrating no correlation between BAMA binding and ADCC titers for WITO, JR-FL and 93MG037.1. Contrary to the correlation observed with 1086c, there was no correlation between BAMA binding and ADCC titers for the other three gp120s which were assessed in both assays.

FIG. 24 depicts the characteristics of acute/early primary Envs. For ease, each plasmid is denoted by the Glade letter followed by a number throughout the paper. All inserts were RNA and codon optimized and encoded for the full gp160 Env protein.

FIG. 25 depicts serum neutralization titers against a panel of tier 2 viruses from the top two rabbits from groups 4, 5, and 6. The two rabbits with the strongest binding titers were tested for neutralization against a panel of Tier 2 viruses. Colors represent the strength of neutralization with green between baseline to 100, yellow 100-200, red 200-500 and deep red great than 500.

### DETAILED DESCRIPTION

The present invention is based in part upon the surprising discovery that delivery of multiple nucleic acid vaccines is able to induce potent antibody dependent cellular cytotoxicity against multiple HIV gp120 and gp140 coated targets. Therefore, the present invention provides compositions and methods for inducing an immune response against HIV. The nucleic acid vaccines described herein can be optimized using the following plasmid-enhancement techniques: codon optimization, RNA optimization, leader sequence addition. The nucleic acid prime can be followed by a protein boost with recombinant HIV gp120.

Groupings or “clouds” of plasmids expressing primary isolate HIV-1 envelopes are able to produce potent anti-envelope antibodies. In addition, priming with a “cloud” expressing primarily transmitted founder envelopes is able to increase the breadth of these responses. In some embodiments, immunized with two primings of the transmitted found envelope “cloud” followed by “clouds” of diverse chronic isolates develop both tier 1 and tier 2 neutralization antibodies which span multiple clades. This robust induction of antibodies has yet to be seen using other platforms and could lend itself well to being further expanded by boosting with other modalities like protein. Many successful anti-viral vaccines have the ability to induce neutralizing antibodies.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

“Adjuvant” as used herein may mean any molecule added to the DNA plasmid vaccines described herein to enhance antigenicity of the one or more immunogens encoded by the DNA plasmids and encoding nucleic acid sequences described hereinafter.

“Antibody” may mean an antibody of classes IgG, IgM, IgA, IgD or IgE, or fragments, fragments or derivatives thereof, including Fab, F(ab′)2, Fd, and single chain antibodies, diabodies, bispecific antibodies, bifunctional antibodies and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Coding sequence” or “encoding nucleic acid” as used herein may mean refers to the nucleic acid (RNA or DNA molecule) that comprise a nucleotide sequence which encodes a protein. The coding sequence may further include initiation and termination signals operably linked to regulatory elements including a promoter and polyadenylation signal capable of directing expression in the cells of an individual or mammal to whom the nucleic acid is administered. In some embodiments, the coding sequence may optionally further comprise a start codon that encodes an N terminal methionine or a signal peptide such as an IgE or IgG signal peptide.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Consensus” or “Consensus Sequence” as used herein may mean a synthetic nucleic acid sequence, or corresponding polypeptide sequence, constructed based on analysis of an alignment of multiple subtypes of a particular antigen. The sequence may be used to induce broad immunity against multiple subtypes or serotypes of a particular antigen. Synthetic antigens, such as fusion proteins, may be manipulated to consensus sequences (or consensus antigens).

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throμghout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drμgs, ions, and water to pass from one side of the cellular membrane to the other.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” or “immunogenic fragment” may mean a polypeptide fragment of an HIV immunogen that is capable of eliciting an immune response in a mammal against HIV by recognizing the particular HIV antigen. The fragment may be capable of eliciting an immune response in a mammal that cross reacts with a full length endogenous antigen. The HIV envelope glycoprotein immunogen may optionally include a signal peptides and/or a methionine at position 1, proteins 98% or more homologous to the consensus sequences set forth herein, proteins 99% or more homologous to the consensus sequences set forth herein, and proteins 100% identical to the sequences set forth herein, in each case with or without signal peptides and/or a methionine at position 1. A fragment may or may not for example comprise a fragment of an HIV immunogen linked to a signal peptide such as an immunoglobulin signal peptide for example IgE signal peptide or IgG signal peptide. Fragments of consensus proteins can comprise at least 10%, at least 20%, at least 30%, at least 40%, at least 50%, at least 60%, at least 70%, at least 80%, at least 90% or at least 95% of a consensus protein. In some embodiments, fragments of consensus proteins can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more, at least 190 amino acids or more, at least 200 amino acids or more, at least 210 amino acids or more, at least 220 amino acids or more, at least 230 amino acids or more, or at least 240 amino acids or more of a consensus protein.

As used herein, the term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acid molecules or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more filovirus consensus antigen via the provided DNA plasmid vaccines. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid molecule may be used for the same purpose as a given nucleic acid molecule. Thus, a nucleic acid molecule also encompasses substantially identical nucleic acids and complements thereof. A single strand provides a probe that may hybridize to a target sequence under stringent hybridization conditions. Thus, a nucleic acid molecule also encompasses a probe that hybridizes under stringent hybridization conditions.

Nucleic acid molecules may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acid molecules may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Signal peptide” as used herein may refer to a short peptide sequence, typically less than 50 amino acids long, which directs the transport of the protein in which it is incorporated. Signal peptides typically are linked to a protein at the N terminus and coding sequences encoding the signal peptide often include the initiation codon that encodes the N terminal methionine encoded by the initiation codon. Signal peptides target the protein for transport within the cell and are involved in the secretory pathway in which the presence of the signal peptide on a protein targets the protein for transport though the secretory pathway such that the protein is secreted by the cell or otherwise targeted for release by the cell into the extracellular environment. In some embodiments, the signal peptide is an immunoglobulin signal peptide such as an IgG or IgE signal peptide. The addition of a coding sequence of a signal peptide to the coding sequences of a protein generally refers to the insertion of the coding sequence of a signal peptide including an initiation codon in place of the initiation codon of the coding sequence of the protein. That is, the addition of the coding sequence of a signal peptide to the coding sequence of the protein involves the removal of the initiation codon of the coding sequence of the protein and the insertion of the coding sequence of a signal peptide including an initiation codon. Thus, in the single peptide plus protein encoded thereby, the methionine at position 1 of the amino acid sequence of the original protein sequence is replaced by the amino acid sequence of the signal peptide which has a methionine at position 1.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5 to 10° C. lower than the thermal melting point (Tm) for the specific sequence at a defined ionic strength pH. The Tm may be the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

A “peptide” or “polypeptide” is a linked sequence of amino acids and can be natural, synthetic, or a modification or combination of natural and synthetic.

“Treatment” or “treating,” when referring to protection of an animal from a disease, means preventing, suppressing, repressing, or completely eliminating the disease. Preventing the disease involves administering a composition of the present invention to an animal prior to onset of the disease. Suppressing the disease involves administering a composition of the present invention to an animal after induction of the disease but before its clinical appearance. Repressing the disease involves administering a composition of the present invention to an animal after clinical appearance of the disease.

“Substantially complementary” as used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” can mean that a first and second amino acid sequence are at least 60%, 65%, 70%, 75%, 80%, 85%, 90%, 95%, 96%, 97%, 98%,or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 nucleotides or amino acids, or with respect to nucleic acids, if the first sequence is substantially complementary to the complement of the second sequence.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Variant” with respect to a nucleic acid sequence that encodes the same specific amino acid sequence differs in nucleotide sequence by use of different codons.

“Vector” used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

“Cloud” is used herein to refer to a formulation of antigens, preferably nucleotide sequences encoding HIV envelope proteins, that can be used to vaccinate a subject. Preferably, each cloud or cloud vaccine is comprised of at least 4 HIV envelope antigens, and more preferably at least 6 HIV envelope antigens. In some embodiments, each cloud is comprised of 6 HIV envelope antigens.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Compositions

Provided herein are HIV immunogens that can be used to induce broad immunity against multiple subtypes or serotypes of a particular HIV antigen. HIV antigens may include sequences of any HIV glycoprotein immunogen. In one embodiment, the immunogen includes a gp160 immunogen. In one embodiment, the immunogen includes a gp120 immunogen. In one embodiment, the immunogen includes a gp41 immunogen. In one embodiment, the immunogen includes Clade A HIV glycoprotein immunogens, Clade B HIV glycoprotein immunogens, or Clade C HIV glycoprotein immunogens.

The immunogens include HIV gp160, HIV gp140, HIV gp120, HIV gp41, and variants thereof, optionally including a signal peptide such as for example an IgE or IgG signal peptide.

In some embodiments, the Env proteins can comprise an amino acid sequence selected from the following list: SEQ ID NOs: 2, 4, 6, 8, 10, 12, 14, 16, 18, 20, 22, 24, 26, 28, 30, 32, 34, 36, 38, 40, 42, 44, 46, 50, 52, 54, 56, or 58.

In some embodiments, the Env proteins can comprise an amino acid sequence that is at least 90% homologous to at least one of SEQ ID NOs: 2, 4, 6, 8, 10, 12, 14, 16, 18, 20, 22, 24, 26, 28, 30, 32, 34, 36, 38, 40, 42, 44, 46, 50, 52, 54, 56, or 58.

In some embodiments, the Env proteins can comprise a fragment of an amino acid sequence selected from the following list: SEQ ID NOs: 2, 4, 6, 8, 10, 12, 14, 16, 18, 20, 22, 24, 26, 28, 30, 32, 34, 36, 38, 40, 42, 44, 46, 50, 52, 54, 56, 58, 60 or 62.

In some embodiments, the Env proteins can comprise a fragment of an amino acid sequence that is at least 90% homologous to at least one of SEQ ID NOs: 2, 4, 6, 8, 10, 12, 14, 16, 18, 20, 22, 24, 26, 28, 30, 32, 34, 36, 38, 40, 42, 44, 46, 50, 52, 54, 56, 58, 60 or 62.

In some embodiments, the vaccination of a subject can further include a HIV pol antigen, for example a HIV pol antigen comprising the amino acid sequence of SEQ ID NO: 48, an amino acid sequence at least 90% homologous to SEQ ID NO: 48, or fragments thereof.

Also provided herein is a composition comprising two or more nucleic acid molecules encoding an HIV immunogen. In one embodiment, the nucleic acid may encode a full length HIV immunogen, a fragment of an HIV immunogen, a protein homologous to an HIV immunogen, or a protein homologous to a fragment of an HIV immunogen. Nucleic acid sequence may optionally comprise coding sequences that encode a signal peptide such as for example an IgE or IgG signal peptide.

In one embodiment, the nucleic acid comprises a sequence selected from SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a fragment of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a sequence that is 90% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, or a fragment of a sequence that is 90% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61.

In one embodiment, the nucleic acid comprises a sequence selected from SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a fragment of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a sequence that is 95% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, or a fragment of a sequence that is 95% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61.

In one embodiment, the nucleic acid comprises a sequence selected from SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a fragment of one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, a sequence that is 99% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61, or a fragment of a sequence that is 99% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59,or 61.

In one embodiment, the nucleic acid sequence comprises a sequence that encodes SEQ ID NOs: 2, 4, 6, 8, 10, 12, 14, 16, 18, 20, 22, 24, 26, 28, 30, 32, 34, 36, 38, 40, 42, 44, 46, 52, 54, 56, 58, 60 or 62.

In one embodiment, the nucleic acid sequence comprises a sequence that encodes a sequence at least 90% homologous to SEQ ID NOs: 2, 4, 6, 8, 10, 12, 14, 16, 18, 20, 22, 24, 26, 28, 30, 32, 34, 36, 38, 40, 42, 44, 46, 52, 54, 56, 58, 60 or 62.

In one embodiment, the nucleic acid comprises a sequence encoding a transmitted founder HIV immunogen. In one embodiment, the nucleic acid comprises a sequence encoding a consensus HIV immunogen. Consensus HIV immunogens are described in PCT Patent Application No. WO2008/014521, the contents of which is fully incorporated by reference.

Compositions are provided which comprise nucleic acid molecules. The compositions may comprise a plurality of copies of a single nucleic acid molecule such a single plasmid, a plurality of copies of two or more different nucleic acid molecules such as two or more different plasmids. For example a composition may comprise plurality of two, three, four, five, six, seven, eight, nine or ten or more different nucleic acid molecules. Such compositions may comprise plurality of two, three, four, five, six, or more different plasmids.

Compositions may comprise nucleic acid molecules, such as plasmids, that collectively contain coding sequence for a single HIV immunogen selected from the group consisting of one or more of a HIV gp160 envelope glycoprotein immunogen, one or more of a HIV gp120 envelope glycoprotein immunogen, one or more of a HIV gp140 envelope glycoprotein immunogen, and one or more of a HIV gp41 envelope glycoprotein immunogen.

Compositions comprise nucleic acid sequence that encode the combination of: one or more of a HIV gp160 envelope glycoprotein immunogen, one or more of a HIV gp120 envelope glycoprotein immunogen, one or more of a HIV gp140 envelope glycoprotein immunogen, and one or more of a HIV gp41 envelope glycoprotein immunogen.

Each coding sequence for each HIV immunogens is preferably included on a separate nucleic acid molecule.

In one embodiment, the composition comprises a plurality of nucleic acid sequences described herein. In one embodiment, the composition comprises 3 or more nucleic acid sequences. In one embodiment, the composition comprises 6 or more nucleic acid sequences. In one embodiment, the composition comprises 10 or more nucleic acid sequences. In one embodiment, the composition comprises 14 or more nucleic acid sequences. In one embodiment, the composition comprises 20 or more nucleic acid sequences. In one embodiment, the composition comprises 25 or more nucleic acid sequences. In one embodiment, the composition comprises 30 or more nucleic acid sequences. In one embodiment, the composition comprises 35 or more nucleic acid sequences. In one embodiment, the composition comprises 40 or more nucleic acid sequences. In one embodiment, the composition comprises two or more nucleic acid molecules, wherein each nucleic acid molecule comprises only one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, or 61.

In one embodiment, the composition comprises 3 or more nucleic acid sequences, where the 3 or more nucleic acid sequences may be on a single nucleic acid molecule or on two nucleic acid molecules in any permutation, but are preferably on three separate nucleic acid molecules (e.g., three separate plasmids).

In one embodiment, the composition comprises 6 or more nucleic acid molecules, where the 6 or more nucleic acid molecules may be on a single plasmid or on two plasmids in any permutation, or on three plasmids in any permutation or on four plasmids in any permutation or on five plasmids in any permutation or, but are preferably on six separate plasmids.

In one embodiment, the composition comprises 10 or more nucleic acid molecules, where the 10 or more nucleic acid molecules be on a single plasmid or on two plasmids in any permutation, or on three plasmids in any permutation or on four plasmids in any permutation or on five plasmids in any permutation or on six plasmids in any permutation, on seven plasmids in any permutation, on eight plasmids in any permutation, on nine plasmids in any permutation, but are preferably on ten separate plasmids.

In one embodiment, the composition comprises 14 or more nucleic acid molecules, where the 14 or more nucleic acid molecules be on a single plasmid or on two plasmids in any permutation, or on three plasmids in any permutation or on four plasmids in any permutation or on five plasmids in any permutation or on six plasmids in any permutation, on seven plasmids in any permutation, on eight plasmids in any permutation, on nine plasmids in any permutation, on ten plasmids in any permutation, on eleven plasmids in any permutation, on twelve plasmids in any permutation, on thirteen plasmids in any permutation, on fourteen plasmids in any permutation, but are preferably on one plasmid or on fourteen plasmids in any permutation .

The compositions can induce potent antibody dependent cellular cytotoxicity (ADCC) against multiple gp120 and gp140 coated targets. The combination of two or more nucleic acid molecules efficiently induces cellular and humoral responses better than one nucleic acid alone.

a. Antigen

The composition may comprise an antigen. The antigen is encoded by a nucleic acid sequence. The nucleic acid sequence may be DNA or RNA. The nucleic acid may encode an antigen or a variant thereof. The antigen can be an antigen isolated from human immunodeficiency virus (HIV). The HIV antigens can include modified consensus sequences for immunogens. Genetic modifications including codon optimization, RNA optimization, and the addition of a high efficient immunoglobin leader sequence to increase the immunogenicity of constructs can be included in the modified consensus sequences. The novel immunogens can be designed to elicit stronger and broader cellular immune responses than a corresponding codon optimized immunogens.

In one embodiment, the antigen encoded by an optimized consensus sequence is capable of eliciting an immune response in a mammal. In one embodiment, the antigen encoded by an optimized consensus sequence can comprise an epitope(s) that makes it particularly effective as an immunogen against which an immune response can be induced.

The optimized consensus sequence can be a consensus sequence derived from two or more native HIV proteins or two or more HIV subtypes. The optimized consensus sequence can comprise a consensus sequence and/or modification(s) for improved expression. Modification can include codon optimization, RNA optimization, addition of a kozak sequence for increased translation initiation, and/or the addition of an immunoglobulin leader sequence to increase immunogenicity. The HIV antigen encoded by the optimized consensus sequence can comprise a signal peptide such as an immunoglobulin signal peptide, for example, but not limited to, an immunoglobulin E (IgE) or immunoglobulin (IgG) signal peptide. In some embodiments, the antigen encoded by the optimized consensus sequence can comprise a hemagglutinin (HA) tag. The HIV antigen encoded by the optimized consensus sequence can be designed to elicit stronger cellular and/or humoral immune responses than a corresponding native antigen.

The antigen of the first vaccine may be the same antigen across different subtypes of HIV. The composition may comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more nucleic acid sequences encoding a particular protein sequence isolated from HIV subtypes A, B, C, D, or other HIV subtypes, or a combination or variant thereof.

In some embodiments, the HIV antigen can be a subtype A consensus envelope DNA sequence construct, an IgE leader sequence linked to a consensus sequence for Subtype A envelope protein, or a subtype A consensus Envelope protein sequence.

In other embodiments, the HIV antigen can be a subtype B consensus envelope DNA sequence construct, an IgE leader sequence linked to a consensus sequence for Subtype B envelope protein, or a subtype B consensus Envelope protein sequence.

In still other embodiments, the HIV antigen can be a subtype C consensus envelope DNA sequence construct, an IgE leader sequence linked to a consensus sequence for subtype C envelope protein, or a subtype C consensus envelope protein sequence.

In further embodiments, the HIV antigen can be a subtype D consensus envelope DNA sequence construct, an IgE leader sequence linked to a consensus sequence for Subtype D envelope protein, or a subtype D consensus envelope protein sequence.

In some embodiments, the HIV antigen can be a subtype A Nef-Rev consensus envelope DNA sequence construct, an IgE leader sequence linked to a consensus sequence for Subtype A Nef-Rev protein, or a Subtype A Nef-Rev consensus protein sequence.

In some embodiments, the HIV antigen can be a subtype B Nef-Rev consensus envelope DNA sequence construct, an IgE leader sequence linked to a consensus sequence for Subtype B Nef-Rev protein, or a Subtype B Nef-Rev consensus protein sequence.

In some embodiments, the HIV antigen can be a subtype C Nef-Rev consensus envelope DNA sequence construct, an IgE leader sequence linked to a consensus sequence for Subtype C Nef-Rev protein, or a Subtype C Nef-Rev consensus protein sequence.

In some embodiments, the HIV antigen can be a subtype D Nef-Rev consensus envelope DNA sequence construct, an IgE leader sequence linked to a consensus sequence for Subtype D Nef-Rev protein, or a Subtype D Nef-Rev consensus protein sequence.

In other embodiments, the HIV antigen can be a Gag consensus DNA sequence of subtype A, B, C and D DNA sequence construct, an IgE leader sequence linked to a consensus sequence for Gag consensus subtype A, B, C and D protein, or a consensus Gag subtype A, B, C and D protein sequence.

In still other embodiments, the HIV antigen can be a MPol DNA sequence or a MPol protein sequence. The HIV antigen can be nucleic acid or amino acid sequences of Env A, Env B, Env C, Env D, B Nef-Rev, Gag, or any combination thereof.

In other embodiments, the HIV antigen may be a DNA sequence or consensus sequence of subtype A, B, C, or D encoding gp140 or consensus gp140 protein. In other embodiments, the HIV antigen may be a DNA sequence or consensus sequence of subtype A, B, C, or D encoding gp140 or consensus gp120 protein. In other embodiments, the HIV antigen gp140 peptide sequence or gp140 consensus peptide sequence of subtype A, B, C, or D. In other embodiments, the HIV antigen gp120 peptide sequence or gp140 consensus peptide sequence of subtype A, B, C, or D. In some embodiments, the HIV antigen gp160 peptide sequence or gp160 consensus peptide sequence of subtype A, B, C, or D.

The antigen can affect a mammal, which can be a human, chimpanzee, dog, cat, horse, cow, mouse, or rat. The antigen can be contained in a protein from a mammal, which can be a human, chimpanzee, dog, cat, horse, cow, pig, sheep, mouse, or rat.

b. DNA

The composition may comprise DNA. Also provided herein is a DNA that encodes the antigen as described above. The DNA can include an encoding sequence that encodes the antigen. The DNA can also include additional sequences that encode linker or tag sequences that are linked to the antigen by a peptide bond.

c. RNA

The composition may comprise RNA. Also provided herein is a RNA that encodes the antigen as described above. The RNA can include an encoding sequence that encodes the antigen. The RNA can also include additional sequences that encode linker or tag sequences that are linked to the antigen by a peptide bond.

d. Vector

The composition may comprise a vector. Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome

The vector can be capable of expressing the antigen. The vector may be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors may have expression signals such as a strong promoter, a strong termination codon, adjustment of the distance between the promoter and the cloned gene, and the insertion of a transcription termination sequence and a PTIS (portable translation initiation sequence).

i. Expression vectors

The vector may be circular plasmid or a linear nucleic acid vaccine. The circular plasmid and linear nucleic acid are capable of directing expression of a particular nucleotide sequence in an appropriate subject cell. The vector may have a promoter operably linked to the antigen-encoding nucleotide sequence, which may be operably linked to termination signals. The vector may also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development.

ii. RNA Vectors

In one embodiment, the nucleic acid is an RNA molecule. Accordingly, in one embodiment, the invention provides an RNA molecule encoding one or more HIV antigens. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

iii. Circular and Linear Vectors

The vector may be circular plasmid, which may transform a target cell by integration into the cellular genome or exist extrachromosomally (e.g. autonomous replicating plasmid with an origin of replication).

The vector can be pVAX, pcDNA3.0, or provax, or any other expression vector capable of expressing the DNA and enabling a cell to translate the sequence to a antigen that is recognized by the immune system. The vector can be combined with antigen at a mass ratio of between 5:1 and 1:5, or of between 1:1 and 2:1.

Plasmid may comprise a nucleic acid sequence that encodes one or more of the various immunogens disclosed above including coding sequences that encode synthetic, consensus antigen capable of eliciting an immune response against HIV immunogens.

A single plasmid may contain coding sequence for a single HIV immunogen, coding sequence for two HIV immunogens, coding sequence for three HIV immunogens, coding sequence for four HIV immunogens, coding sequence for five HIV immunogens or coding sequence for six HIV immunogens. A single plasmid may contain a coding sequence for a single HIV immunogen which can be formulated together. In some embodiments, a plasmid may comprise coding sequence that encodes IL-12, IL-15 and/or IL-28.

The plasmid may further comprise an initiation codon, which may be upstream of the coding sequence, and a stop codon, which may be downstream of the coding sequence. The initiation and termination codon may be in frame with the coding sequence.

The plasmid may also comprise a promoter that is operably linked to the coding sequence The promoter operably linked to the coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, or human metalothionein. The promoter may also be a tissue specific promoter, such as a muscle or skin specific promoter, natural or synthetic. Examples of such promoters are described in US patent application publication no. US20040175727, the contents of which are incorporated herein in its entirety.

The plasmid may also comprise a polyadenylation signal, which may be downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

The plasmid may also comprise an enhancer upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may also comprise a regulatory sequence, which may be well suited for gene expression in a cell into which the plasmid is administered. The coding sequence may comprise a codon that may allow more efficient transcription of the coding sequence in the host cell.

The coding sequence may also comprise an Ig leader sequence. The leader sequence may be 5′ of the coding sequence. The consensus antigens encoded by this sequence may comprise an N-terminal Ig leader followed by a consensus antigen protein. The N-terminal Ig leader may be IgE or IgG.

The plasmid may be pSE420 (Invitrogen, San Diego, Calif.), which may be used for protein production in Escherichia coli (E. coli). The plasmid may also be pYES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNA I or pcDNA3 (Invitrogen, San Diego, Calif.), which may be used for protein production in mammalian cells such as Chinese hamster ovary (CHO) cells.

Also provided herein is a linear nucleic acid vaccine, or linear expression cassette (“LEC”), that is capable of being efficiently delivered to a subject via electroporation and expressing one or more desired antigens. The LEC may be any linear DNA devoid of any phosphate backbone. The DNA may encode one or more antigens. The LEC may contain a promoter, an intron, a stop codon, a polyadenylation signal. The expression of the antigen may be controlled by the promoter. The LEC may not contain any antibiotic resistance genes and/or a phosphate backbone. The LEC may not contain other nucleic acid sequences unrelated to the desired antigen gene expression.

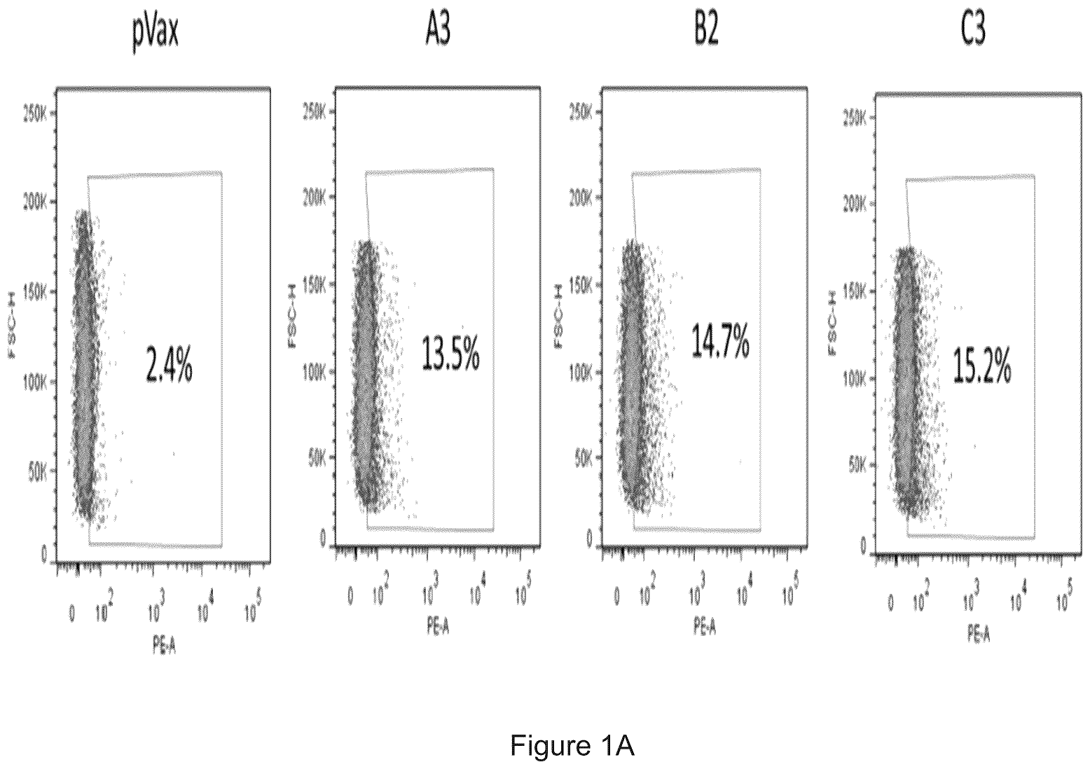

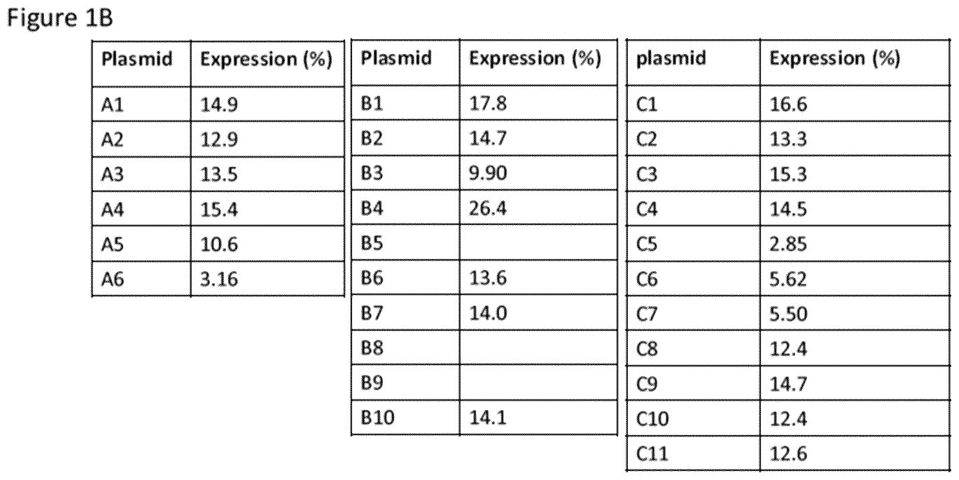

The LEC may be derived from any plasmid capable of being linearized. The plasmid may be capable of expressing the antigen. The plasmid may be pNP (Puerto Rico/34) or pM2 (New Caledonia/99). See FIG. 1. The plasmid may be pVAX, pcDNA3.0, or provax, or any other expression vector capable of expressing the DNA and enabling a cell to translate the sequence to a antigen that is recognized by the immune system.

The LEC may be pcrM2. The LEC may be pcrNP. pcrNP and pcrMR may be derived from pNP (Puerto Rico/34) and pM2 (New Caledonia/99), respectively. The LEC may be combined with antigen at a mass ratio of between 5:1 and 1:5, or of between 1:1 to 2:1.

iv. Promoter, Intron, Stop Codon, and Polyadenylation Signal

The vector may have a promoter. A promoter may be any promoter that is capable of driving gene expression and regulating expression of the isolated nucleic acid. Such a promoter is a cis-acting sequence element required for transcription via a DNA dependent RNA polymerase, which transcribes the antigen sequence described herein. Selection of the promoter used to direct expression of a heterologous nucleic acid depends on the particular application. The promoter may be positioned about the same distance from the transcription start in the vector as it is from the transcription start site in its natural setting. However, variation in this distance may be accommodated without loss of promoter function.

The promoter may be operably linked to the nucleotide sequence encoding the antigen and signals required for efficient polyadenylation of the transcript, ribosome binding sites, and translation termination. The promoter may be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

The vector may include an enhancer and an intron with functional splice donor and acceptor sites. The vector may contain a transcription termination region downstream of the structural gene to provide for efficient termination. The termination region may be obtained from the same gene as the promoter sequence or may be obtained from different genes.

e. Vaccines

Provided herein is a vaccine capable of generating in a mammal an immune response against HIV. The vaccine may comprise each plasmid as discussed above. The vaccine may comprise a plurality of the plasmids, or combinations thereof. The vaccine may be provided to induce a therapeutic or prophylactic immune response.

Vaccines may be used to deliver nucleic acid molecules that encode consensus HIV envelope glycoprotein immunogens. Vaccines may be used to deliver nucleic acid molecules that encode transmitted founder HIV envelope glycoprotein immunogens. Vaccines may be used to deliver nucleic acid molecules that encode consensus Clade A, Clade B, Clade C, or Clade D HIV envelope glycoprotein immunogens. Vaccines may be used to deliver nucleic acid molecules that encode transmitted founder Clade A, Clade B, Clade C, or Clade D HIV envelope glycoprotein immunogens. Vaccines may be used to deliver nucleic acid molecules that encode consensus HIV gp160 envelope glycoprotein immunogens. Vaccines may be used to deliver nucleic acid molecules that encode transmitted founder HIV gp160 envelope glycoprotein immunogens. Vaccines may be used to deliver nucleic acid molecules that encode consensus HIV gp140 envelope glycoprotein immunogens. Vaccines may be used to deliver nucleic acid molecules that encode transmitted founder HIV gp140 envelope glycoprotein immunogens. Vaccines may be used to deliver nucleic acid molecules that encode consensus HIV gp120 envelope glycoprotein immunogens. Vaccines may be used to deliver nucleic acid molecules that encode transmitted founder HIV gp120 envelope glycoprotein immunogens. Vaccines may be used to deliver nucleic acid molecules that encode consensus HIV gp41 envelope glycoprotein immunogens. Vaccines may be used to deliver nucleic acid molecules that encode transmitted founder HIV gp41 envelope glycoprotein immunogens.

The vaccine may comprise the antigens and plasmids at quantities of from about 1 nanogram to 100 milligrams; about 1 microgram to about 10 milligrams; or preferably about 0.1 microgram to about 10 milligrams; or more preferably about 1 milligram to about 2 milligram. In some preferred embodiments, pharmaceutical compositions according to the present invention comprise about 5 nanogram to about 1000 micrograms of DNA. In some preferred embodiments, the pharmaceutical compositions contain about 10 nanograms to about 800 micrograms of DNA. In some preferred embodiments, the pharmaceutical compositions contain about 0.1 to about 500 micrograms of DNA. In some preferred embodiments, the pharmaceutical compositions contain about 1 to about 350 micrograms of DNA. In some preferred embodiments, the pharmaceutical compositions contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of the consensus antigen or plasmid thereof.

f. Other Components of Vaccine-Adjuvants, Excipients

The composition may further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate is may be present in the vaccine at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. The DNA plasmid vaccines may also include a transfection facilitating agent such as lipids, liposomes, including lecithin liposomes or other liposomes known in the art, as a DNA-liposome mixture (see for example W09324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in alternative plasmid or are delivered as proteins in combination with the plasmid above in the vaccine. The adjuvant may be selected from the group consisting of: α-interferon(IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80,CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes that can be useful adjuvants include those encoding: MCP-1, MIP-la, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The composition may further comprise a genetic vaccine facilitator agent as described in U.S. Ser. No. 021,579 filed Apr. 1, 1994, which is fully incorporated by reference.

The composition can be formulated according to the mode of administration to be used. An injectable composition pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polyanions or polyanions.

### 2. Method of Vaccination

Provided herein is a method for delivering the vaccine for providing genetic constructs and proteins of the antigen which comprise epitopes that make them particular effective against immunogens of HIV, against which an immune response can be induced. The method of delivering the vaccine or vaccination may be provided to induce a therapeutic and prophylactic immune response. The vaccination process may generate in the mammal an immune response against HIV. The vaccine may be delivered to an individual to modulate the activity of the mammal's immune system and enhance the immune response. The delivery of the vaccine may be the transfection of the antigen as a nucleic acid molecule that is expressed in the cell and delivered to the surface of the cell upon which the immune system recognized and induces a cellular, humoral, or cellular and humoral response. The delivery of the vaccine may be used to induce or elicit and immune response in mammals against HIV by administering to the mammals the vaccine as discussed above.

Upon delivery of the vaccine and plasmid into the cells of the mammal, the transfected cells will express and secrete consensus antigens for each of the plasmids injected from the vaccine. These proteins will be recognized as foreign by the immune system and antibodies will be made against them. These antibodies will be maintained by the immune system and allow for an effective response to subsequent infections by HIV.

Also provided herein is a method of immunizing a subject against HIV to treat or prevent HIV infection using the composition. The method of immunizing a subject comprises administering a first composition comprising one or more nucleic acid molecules encoding a sequence at least 90% homologous to SEQ ID NOs: 2, 4, 6, 8, 10, 12, 14, 16, 18, 20, 22, 24, 26, 28, 30, 32, 34, 36, 38, 40, 42, 44, 46, 52, 54, 56, 58, 60, or 62, a variant thereof or a fragment thereof. In one embodiment, the first composition comprises one or more nucleic acid molecules having a sequence at least 90% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, or 61. The first composition may be given in multiple doses. In one embodiment, the first composition is administered twice. The first composition can be administered a second time within 2 days, 5 days, or 7 days of the first administration of the first composition. In one embodiment, the first composition is administered intradermally. The first composition can efficiently deliver antigen to a subject in need thereof for immune stimulation via a priming dose.

In one embodiment, the method further comprises administering a second composition comprising one or more nucleic acid molecules encoding a sequence at least 90% homologous to SEQ ID NOs: 2, 4, 6, 8, 10, 12, 14, 16, 18, 20, 22, 24, 26, 28, 30, 32, 34, 36, 38, 40, 42, 44, 46, 52, 54, 56, 58, 60, or 62, a variant thereof or a fragment thereof. In one embodiment, the second composition comprises one or more nucleic acid molecules having a sequence at least 90% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, or 61. In one embodiment, the second composition comprises nucleic acid molecules different than the nucleic acid molecules comprised within the first composition. In one embodiment, the second composition is administered at least 3 or more, 6 or more, or 12 or more weeks after the first composition is administered. The second composition may be given in multiple doses. In one embodiment, the second composition is administered twice. The second composition can be administered a second time within 1 week, 2 weeks, 4 weeks or 6 weeks of the first administration of the composition. In one embodiment the second composition is administered intradermally.

In one embodiment, the method further comprises administering a third composition comprising one more nucleic acid molecules encoding a sequence at least 90% homologous to SEQ ID NOs: 2, 4, 6, 8, 10, 12, 14, 16, 18, 20, 22, 24, 26, 28, 30, 32, 34, 36, 38, 40, 42, 44, 46, 52, 54, 56, 58, 60, or 62, a variant thereof or a fragment thereof. In one embodiment, the third composition comprises one or more nucleic acid molecules having a sequence at least 90% homologous to one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, or 61 In one embodiment, the third composition comprises each nucleic acid comprised in the first composition and the second composition. In one embodiment, the third composition is administered at least 10 or more, 15 or more, 20 or more or 25 or more weeks after the second composition is administered. The third composition may be given in multiple doses. In one embodiment, the third composition is administered twice. The third composition can be administered a second time within 25 weeks, 30 weeks, or 40 weeks of the first administration of the third composition. In one embodiment, the second composition is administered intramuscularly.

The number of composition doses for effective treatment can be 1, 2, 3, 4, 5, 6, 7, 8, 9, or 10.

The composition may be administered to a mammal to elicit an immune response in a mammal. The mammal may be human, primate, non-human primate, cow, cattle, sheep, goat, antelope, bison, water buffalo, bison, bovids, deer, hedgehogs, elephants, llama, alpaca, mice, rats, and chicken.

a. Immune Response

The composition can induce an immune response in the subject administered the composition. The induced immune response can be specific for a native antigen. The induced immune response can be reactive with a native antigen related to the optimized consensus-encoded antigen. In various embodiments, related antigens include antigens having amino acid sequences having at least 90%, at least 91%, at least 92%, at least 93%, at least 94%, at least 95%, at least 96%, at least 97%, at least 98%, at least 99%, or 100% homology to the amino acid sequence of the optimized consensus-encoded antigen. In various embodiments, related antigens include antigens encoded by nucleotide sequences having at least 90%, at least 91%, at least 92%, at least 93%, at least 94%, at least 95%, at least 96%, at least 97%, at least 98%, at least 99%, or 100% homology to the optimized consensus nucleotide sequences disclosed herein.

The immunogenic composition can induce a humoral immune response in the subject administered the immunogenic composition. The induced humoral immune response can be specific for a native antigen. The induced humoral immune response can be reactive with the native antigen related to the optimized consensus-encoded antigen. The humoral immune response can be induced in the subject administered the immunogenic composition by about 1.5-fold to about 16-fold, about 2-fold to about 12-fold, or about 3-fold to about 10-fold. The humoral immune response can be induced in the subject administered the immunogenic composition by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0- fold as compared to a subject not administered the immunogenic composition or a subject administered a non-optimized antigen.

The humoral immune response induced by the immunogenic composition can include an increased level of neutralizing antibodies associated with the subject administered the immunogenic composition as compared to a subject not administered the immunogenic composition. The neutralizing antibodies can be specific for a native antigen related to the optimized consensus-encoded antigen. The neutralizing antibodies can be reactive with the native antigen genetically related to the optimized consensus antigen. The neutralizing antibodies can provide protection against and/or treatment of tumor growth, metastasis or tumor associated pathologies in the subject administered the immunogenic composition.

The humoral immune response induced by the immunogenic composition can include an increased level of IgG antibodies associated with the subject administered the immunogenic composition as compared to a subject not administered the immunogenic composition. These IgG antibodies can be specific for the native antigen genetically related to the optimized consensus antigen. These IgG antibodies can be reactive with the native antigen genetically related to the optimized consensus antigen. The level of IgG antibody associated with the subject administered the immunogenic composition can be increased by about 1.5-fold to about 16-fold, about 2-fold to about 12-fold, or about 3-fold to about 10-fold as compared to the subject not administered the immunogenic composition. The level of IgG antibody associated with the subject administered the immunogenic composition can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to a subject not administered the immunogenic composition or a subject administered a non-optimized antigen.

The immunogenic composition can induce a cellular immune response in the subject administered the immunogenic composition. The induced cellular immune response can be specific for a native antigen related to the optimized consensus-encoded antigen. The induced cellular immune response can be reactive to the native antigen related to the optimized consensus-encoded antigen. The induced cellular immune response can include eliciting a CD8⁺ T cell response. The elicited CD8⁺ T cell response can be reactive with the native antigen genetically related to the optimized consensus antigen. The elicited CD8⁺ T cell response can be polyfunctional. The induced cellular immune response can include eliciting a CD8⁺ T cell response, in which the CD8⁺ T cells produce interferon-gamma (IFN-γ), tumor necrosis factor alpha (TNF-α), interleukin-2 (IL-2), or a combination of IFN-γ and TNF-α.

The induced cellular immune response can include an increased CD8⁺ T cell response associated with the subject administered the immunogenic composition as compared to the subject not administered the immunogenic composition. The CD8⁺ T cell response associated with the subject administered the immunogenic composition can be increased by about 2-fold to about 30-fold, about 3-fold to about 25-fold, or about 4-fold to about 20-fold as compared to the subject not administered the immunogenic composition. The CD8⁺T cell response associated with the subject administered the immunogenic composition can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to a subject not administered the immunogenic composition or a subject administered a non-optimized antigen.

The induced cellular immune response can include an increased frequency of CD107a/IFNγ/T-bet triple-positive CD8 T cells that are reactive against the native antigen. The frequency of CD107a/IFNγ/T-bet triple-positive CD8 T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to a subject not administered the immunogenic composition or a subject administered a non-optimized antigen.

The induced cellular immune response can include an increased frequency of CD107a/IFNγ double-positive CD8 T cells that are reactive against the native antigen. The frequency of CD107a/IFNγ double-positive CD8 T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, or 14-fold as compared to a subject not administered the immunogenic composition or a subject administered a non-optimized antigen.

The cellular immune response induced by the immunogenic composition can include eliciting a CD4⁺ T cell response. The elicited CD4⁺ T cell response can be reactive with the native antigen genetically related to the optimized consensus antigen. The elicited CD4⁺ T cell response can be polyfunctional. The induced cellular immune response can include eliciting a CD4⁺ T cell response, in which the CD4⁺ T cells produce IFN-γ, TNF-α, IL-2, or a combination of IFN-γ and TNF-α.

The induced cellular immune response can include an increased frequency of CD4⁺ T cells that produce IFN-γ. The frequency of CD4⁺IFN-γ⁺ T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to a subject not administered the immunogenic composition or a subject administered a non-optimized antigen.

The induced cellular immune response can include an increased frequency of CD4⁺ T cells that produce TNF-α. The frequency of CD4⁺TNF-α⁺ T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, or 22-fold as compared to a subject not administered the immunogenic composition or a subject administered a non-optimized antigen.

The induced cellular immune response can include an increased frequency of CD4⁺ T cells that produce both IFN-γ and TNF-α. The frequency of CD4⁺IFN-γ⁺TNF-α⁺ associated with the subject administered the immunogenic composition can be increased by at least about 2-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 22-fold, 23-fold 24-fold, 25-fold, 26-fold, 27-fold, 28-fold, 29-fold, 30-fold, 31-fold, 32-fold, 33-fold, 34-fold, or 35-fold as compared to a subject not administered the immunogenic composition or a subject administered a non-optimized antigen.

The immunogenic composition of the present invention can have features required of effective vaccines such as being safe so the vaccine itself does not cause illness or death; is protective against illness resulting from exposure to live pathogens such as viruses or bacteria; induces neutralizing antibody to prevent invention of cells; induces protective T cells against intracellular pathogens; and provides ease of administration, few side effects, biological stability, and low cost per dose.

The immunogenic composition can further induce an immune response when administered to different tissues such as the muscle or skin. The immunogenic composition can further induce an immune response when administered via electroporation, or injection, or subcutaneously, or intramuscularly.

b. Cloud Vaccines

The cloud vaccines can include HIV antigens, and preferably Env and more preferably Env of Clade A, Clade B, or Clade C. It is preferable to have a cloud vaccine comprised of the nucleotide sequences encoding an Env protein described herein.

Could vaccines can be comprised of one of more of the Env encoding nucleotide sequences, and can comprise at least 4, at least 5, at least 6, at least 7, at least 8, at least 9, at least 10, at least 11, or at least 12 Env.

The cloud vaccines can be one of the following groups of antigens in each cloud (referring to table 1a and 1b, below, for abbreviations):
a. A1, A2, A3, A4, A5, and A6
b. B3, B4, B8, and B9
c. B1, B2, B5, B6, and B7
d. C1, C3, C5, C6, and C7
e. A1, A2, A3, and A4
f. A2, A3, A4, and A5
g. A3, A4, A5, and A6
h. A1, A2, A3, A4, and A5
i. A2, A3, A4, A5, and A6
j. B1, B2, B3, B4, B5, and B6
k. B2, B3, B4, B5, B6 and B7
l. B3, B4, B, B5, B6, B7, and B8
m. B4, B5, B6, B7, B8, and B9
n. B5, B6, B7, B8, B9, and B10
o. B1, B2, B3, B4, B5, B6, and B7
p. B1, B2, B3, B4, B5, B6, B7, and B8
q. B1, B2, B3, B4, B5, B6, B7, B8, and B9
r. B1, B2, B3, B4, B5, B6, B7, B8, B9, and B10
s. B2, B3, B4, B5, B6, B7, B8, and B9
t. B2, B3, B4, B5, B6, B7, B8, B9, and B10
u. B3, B4, B5, B6, B7, B8, and B9
v. B3, B4, B5, B6, B7, B8, B9, and B10
w. C1, C2, C3, C4, C5, and C6
x. C2, C3, C4, C5, C6, and C7
y. C3, C4, C5, C6, C7, and C8
z. C4, C5, C6, C7, C8, and C9
aa. C5, C6, C7, C8, C9, and C10
bb. C6, C7, C8, C9, C10, and C11
cc. C1, C2, C3, C4, C5, C6, and C7
dd. C1, C2, C3, C4, C5, C6, C7, and C8
ee. C1, C2, C3, C4, C5, C6, C7, C8 and C9
ff. C1, C2, C3, C4, C5, C6, C7, C8, C9, and C10
gg. C1, C2, C3, C4, C5, C6, C7, C8, C9, C10, and C11
hh. C2, C3, C4, C5, C6, C7, and C8
ii. C2, C3, C4, C5, C6, C7, C8 and C9
jj. C2, C3, C4, C5, C6, C7, C8, C9, and C10
kk. C2, C3, C4, C5, C6, C7, C8, C9, C10, and C11
ll. C3, C4, C5, C6, C7, C8, and C9
mm. C3, C4, C5, C6, C7, C8, C9, and C10
nn. C3, C4, C5, C6, C7, C8, C9, C10, and C11
oo. C4, C5, C6, C7, C8, C9 and C10
pp. C4, C5, C6, C7, C8, C9, C10 and C11
qq. C5, C6, C7, C8, C9, C10, and C11
rr. C6, C7, C8, C9, C10, and C11
and other subcombinations of the groups, above.

The clouds will have Env antigens that are closely related, and preferably of the same Glade. Preferably, within each cloud the diversity between Env antigens (intra-cloud diversity) is between 10% and 20%, preferably 12% and 18%; more preferably between 12% and 17%; between 12% and 16%; between 13% and 18%; between 13% and 17%; between 13% and 16%; between 14% and 18%; or between 14% and 17%.

Preferably, the diversity between Env antigens between clouds (inter-cloud diversity) between 12% and 25%, preferably 13% and 25%; more preferably between 14% and 25%; between 14% and 24%; between 14% and 23%; between 14% and 22%; between 14% and 21%; between 15% and 22%; or between 15% and 20%.

In some embodiments, the intracloud diversity (within each cloud) ranged from 10-20%, preferably 12.4-16.4% and intercloud diversity (between clouds) was consistently around 20%. The intracloud diversity ranged from 12-16%, preferably 13.3-14.3% and the intercloud diversity between 12-20%, preferably 14-17.6%.

Preferably, the cloud vaccines comprise groups a., b., and c., above. Most preferred is cloud vaccine of group a, above, which is comprised of SEQ ID NOs: 1, 3, 5, 7, 45, and 9.

Cloud Vaccines, above, the vaccination schedule for administering to a mammal can be chosen from the following (Table A):

TABLE A
Vaccination dosing schedules. This table is not meant to be exhaustive.
Other combinations using the clouds described above, are contemplated
using various prime and dose combinations.

| | Priming | | Boost | |
|---|---|---|---|---|
| Vaccination | Number | Cloud | Number | Cloud |
| schedule no. | of dose | No. | Dose | No. |
| i. | 2x | a | 1x; 1x | b; c |
| ii. | 2x | a | 2x | b |
| iii. | 2x | a | 2x | c |
| iv. | 2x | a | 2x | d |
| v. | 2x | a | 2x | e |
| vi. | 2x | a | 2x | f. |
| vii. | 2x | b. | 1x; 1x | a; c |
| viii. | 2x | b. | 2x | a |
| ix. | 2x | b. | 2x | c |
| x. | 2x | b. | 2x | d |
| xi. | 2x | b. | 2x | e. |
| xii. | 2x | c. | 1x; 1x | a; b. |
| xiii. | 2x | c. | 2x | a. |
| xiv. | 2x | c. | 2x | b. |
| xv. | 2x | c. | 2x | d |
| xvi. | 2x | c. | 2x | e. |
| xvii. | 1x; 1x | a; b | 1x; 1x | c.; d. |
| xviii. | 1x; 1x | a; b | 2x | c |
| xix. | 1x; 1x | a; b | 2x | d. |
| xx. | 1x; 1x | a; b | 2x | e |

Preferably, vaccination schedule i. can be used to treat a subject infected with HIV virus.

c. Combination Treatments

The composition may be administered in combination with other proteins and/or genes encoding CCL20, α-interferon, γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15 including IL-15 having the signal sequence deleted and optionally including the different signal peptide such as the IgE signal peptide, MHC, CD80, CD86, IL-28, IL-1, IL-2, IL-4, IL-5, IL-6, IL-10, IL-18, MCP-1, MIP-lα, MIP-1β, IL-8, RANTES, L-selectin, P-selectin, E-selectin, CD34, GlyCAM-1, MadCAM-1, LFA-1, VLA-1, Mac-1, pl50.95, PECAM, ICAM-1, ICAM-2, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof or combinations thereof. In some embodiments, the vaccine is administered in combination with one or more of the following nucleic acid molecules and/or proteins: nucleic acid molecules selected from the group consisting of nucleic acid molecules comprising coding sequence that encode one or more of CCL20, IL-12, IL-15, IL-28, CTACK, TECK, MEC and RANTES or functional fragments thereof, and proteins selected from the group consisting of: CCL02, IL-12 protein, IL-15 protein, IL-28 protein, CTACK protein, TECK protein, MEC protein or RANTES protein or functional fragments thereof.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal, intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The vaccine may be administered by traditional syringes, needleless injection devices, “microprojectile bombardment gone guns”, or other physical methods such as electroporation (“EP”), “hydrodynamic method”, or ultrasound.

d. Administration

The composition can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The composition can be administered prophylactically or therapeutically. In prophylactic administration, the vaccines can be administered in an amount sufficient to induce iTreg responses. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The composition can be administered by methods well known in the art as described in Donnelly et al. (Ann. Rev. Immunol. 15:617-648 (1997)); Feigner et al. (U.S. Pat. No. 5,580,859, issued Dec. 3, 1996); Feigner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The DNA of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The composition can be delivered via a variety of routes. Typical delivery routes include parenteral administration, e.g., intradermal, intramuscular or subcutaneous delivery. Other routes include oral administration, intranasal, and intravaginal routes. For the DNA of the vaccine in particular, the vaccine can be delivered to the interstitial spaces of tissues of an individual (Feigner et al., U.S. Pat. Nos. 5,580,859 and 5,703,055, the contents of all of which are incorporated herein by reference in their entirety). The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The composition can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The composition can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The composition can be incorporated into liposomes, microspheres or other polymer matrices (Feigner et al., U.S. Pat. No. 5,703,055; Gregoriadis, Liposome Technology, Vols. Ito III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The composition can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

The minimally invasive electroporation device (“MID”) may be an apparatus for injecting the vaccine described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the composition into tissue without the use of a needle. The MID may inject the vaccine as a small stream or jet with such force that the vaccine pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. Nos. 6,520,950; 7,171,264; 6,208,893; 6,009,347; 6,120,493; 7,245,963; 7,328,064; and 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired composition in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle vaccine injectors that deliver the vaccine and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA (Inovio Pharmaceuticals, Blue Bell PA) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference.

The MID may be an Elgen 1000 system (Inovio Pharmaceuticals). The Elgen 1000 system may comprise device that provides a hollow needle; and fluid delivery means, wherein the apparatus is adapted to actuate the fluid delivery means in use so as to concurrently (for example automatically) inject fluid, the described vaccine herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present invention include tumor tissue, skin or liver tissue but may be muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so user's have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

e. Method of Preparing DNA Plasmids

Provided herein is methods for preparing the DNA plasmids that comprise the DNA vaccines discussed herein. The DNA plasmids, after the final subcloning step into the mammalian expression plasmid, can be used to inoculate a cell culture in a large scale fermentation tank, using known methods in the art.

The DNA plasmids for use with the EP devices of the present invention can be formulated or manufactured using a combination of known devices and techniques, but preferably they are manufactured using an optimized plasmid manufacturing technique that is described in a licensed, co-pending U.S. application Ser. No. 12/126,611, which was filed on May 23, 2008. In some examples, the DNA plasmids used in these studies can be formulated at concentrations greater than or equal to 10 mg/mL. The manufacturing techniques also include or incorporate various devices and protocols that are commonly known to those of ordinary skill in the art, in addition to those described in U.S. application Ser. No. 12/126,611, including those described in a licensed patent, U.S. Pat. No. 7,238,522, which issued on Jul. 3, 2007. The above-referenced application and patent, U.S. application Ser. No. 12/126,611 and U.S. Pat. No. 7,238,522, respectively, are hereby incorporated in their entirety

The present invention has multiple aspects, illustrated by the following non-limiting examples.

### EXAMPLES

The present invention is further illustrated in the following Example. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

### Materials and Methods

### Envelope Immunogens (Env)

Plasmids expressing codon and RNA optimized HIV-1 envelope glycoproteins (gp160) were made synthetically using OptimumGene Codon optimization analysis (GenScript). Inserts were then cloned into the pVAX (Invitrogen) backbone using either BamHI/XhoI or BamHI/EcoRI cloning sites. Each insert was under the control of the cytomegalovirus immediate-early promoter.

### Expression of Plasmids

Each plasmid was tested in vitro for proper expression. Briefly, HEK 293T cells (ACTC) were cultured in Dulbecco's Modified Eagle Medium (Life Technologies) supplemented with 10% fetal bovin serum and 1% penicillin and streptomycin. Twenty four hours before transfection, 7.5×10⁵ cells were plated in 1.5 mls of media in a 6 well dish. Each plasmid was used in a separate transfection with pVax empty backbone serving as a negative control. Transfection was performed using NeoFectin transfection reagent (NeoBio Labs) following manufactures protocol. Fourty-eight hours after transfection, cells were collected and washed with PBS. Cells were then incubated with 2G12 (Immune Tech) at a 1:100 dilution in Facs buffer (1% FBS in PBS) for 1 hour at room temperature. After washing the cells with PBS, mouse anti-human phycoerythrin linked antibody was added at a 1:5000 dilution for 1 hour at room temperature. Cells were then washed and fixed with 3% paraformaldehyde and run on a modified LSR II (BD Biosciences). Analysis was performed using FlowJo software (FlowJo Enterprise).

### Immunization of Guinea Pigs

Female Hartley guinea pigs (300-350 grams) were immunized with 25 μg of DNA intradermal every 3 weeks with in vivo electroporation using the CELLECTA adaptive constant current electroporation device (Inovio Pharmaceuticals, Blue Bell, PA). Square-wave pulses were delivered with a triangular electrode array consisting of 3 26-gauge solid stainless steel electrodes. Two constant current pulses of 0.2 Amps were delivered with a 3 second delay and 52 ms length. Blood was collected for analysis before every vaccination.

### Immunization of Rabbits

Female New Zealand white rabbits (1900 grams) were immunized using between 100 μg −200 μg/plasmid of DNA intradermal every 3 weeks with in vivo electroporation using the CELLECTA adaptive constant current electroporation device (Inovio Pharmaceuticals, Blue Bell, PA). Group 1 rabbits received 200 μg total of each plasmid delivered to two sites. Group 2 rabbits received 100 μg of each DNA plasmid injected into 6 separate sites followed by electroporation. Groups 3-6 received a mixture of 100 μg/plasmid injected into multiple sites (4-6 depending on the number of plasmids) followed by electroporation. Each site received 100 μg of mixed DNA. Blood was collected for analysis before every vaccination.

### Immunization of Non-Human Primates

Eight Indian rhesus macaques were house at Bioqual (Rockville MD) according to the standards to the American Association for Accreditation of Laboratory Animal Care and all animal protocols were IACUC approved. All animals received six vaccinations: the first four were administered intradermally, and the last two were administered intramuscularly. The first and second vaccination on weeks 0 and 6 were a combination of five Glade A primary envelopes (1.0 mgs each), formulated together and delivered to 5 separate sites. The third immunization delivered on week 12 was a combination of four Glade B envelopes (1.0 mgs each), formulated together and administered to four different sites. The four immunization delivered on week 18 was a combination of six Glade B envelopes (1.0 mgs each), formulated together and administered to six different sites. The fifth and six vaccination were given on weeks 44 and 81, composed of all 15 envelopes (1.0 mgs each) formulated together and delivered to a single site. All DNA deliveries were followed by in vivo EP with the constant current CELLECTRA® device (Inovio Pharmaceuticals, Plymouth Meeting, PA) with 3 pulses at 0.5A constant current, a 52 ms pulse length and 1s rest between pulses.

### Blood Collection

Animals were bled 2 weeks following each immunization. Blood (15 ml at each time point) was collected in EDTA tubes and peripheral blood mononuclear cells (PBMCs) were isolated using standard Ficoll-Hypaque procedure with Accuspin tubes (Sigma-Aldrich, St. Louis MO). An additional 10 ml was collected into clot tubes for serum collection.

Rhesus IFN-Gamma Enzyme-Linked Immunospot Assay (ELISpot). To determine cellular responses, interferon-gamma (IFN-γ) ELISpots (MabTech, Stockholm Sweden) were performed following manufactures protocols. Isolated PBMCs were stimulated overnight in the presence of either specific peptide antigens (Consensus Glade A and B envelope peptides (NIH AIDS Research & Reagent Program, Germantown, MD), R10 (negative control), or anti-CD3 (positive control). All samples were run in triplicate.

### Endpoint Binding Titer ELISA

The measurement of anti-HIV gp120 specific antibodies was determined by ELISA (enzyme linked immunosorbent assay). Nunc-Immuno Plates (Nalge Nunc Internaltional) were coated with 1 μg/ml of either consensus Glade A, 92RW020, SF162, or ZM197M soluble gp120 (Immune Technology Corp) and incubated overnight at 4 deg C. After washing, plates were block with 10% fetal bovine serum (FBS) in lx phosphate-buffered saline (PBS) for 1 hour at room temperature. Plates were then washed again and incubated with specific guinea pig or rabbit sera diluted with 1% FBS in 1×PBS+0.02% Tween-20 for 1 hour at room temperature. After washing, plates were incubated with 1/2,000 or 1/5,000 dilution of horseradish peroxidase-conjugated goat anti-guinea pig or donkey anti-rabbit IgG respectively (Santa Cruz Biotech) for 1 hour at room temperature. The reaction was developed using the SigmaFast OPD tablets and stopped with 100 μ of 2N sulfuric acid/well. Plates were read on Promega Globmax Multi detection system at an OD of 450nm. Endpoint titers were determined as previously reported (Frey et al 1998). Briefly, the upper prediction limit of Envelope specific IgG antibodies was calculated using the Student t distribution. The upper prediction limit was defined as the standard deviation multiplied by a factor based on the number of naive controls and a 95% confidence interval. Endpoint titer was the lowest dilution that remained above the upper prediction limit.

### Epitope Mapping ELISA

Consensus Glade C linear 15-mer peptides with 11 amino acid overlap (NIH AIDS Research and Reference Reagent Program) were used to make pools of the variable regions of gp120 and gp41. Peptides were resuspended in 1×PBS at a concentration of 1 mg/ml of each peptide. Plates were coated with 1 μg/ml of pooled peptides and ELISA was performed as described above. Sera from groups 2, 3, 4, 5 and 6 weeks 0 and 12 were diluted 1/50.

### Neutralization Assay

HIV-1 envelope pseudovirus production and titration was performed as previously described (Seaman et al., 2010, J Virol 84:1439-52). Briefly, single round infectious HIV-1 env pseudoviruess were produced by co-transfection of 293T cells with 2 μg of an HIV-1 env/rev expressing plasmid and 12 μg of HIV-1 Δenv backbone plasmid (pSG3ΔEnv) using Lipofectamine transfection reagent (Invitrogen). After 24 hours, virus containing supernatant was harvested, spun and filtered over a 0.45 μm filter. The 50% tissue culture infectious dose was determined using TZM.bl cells as previously described (Li M et al 2005 J. Virol 79(16):10108-25). Aliquoted pseudotyped virus was stored at −80° C. TZM.bl cells were used to determine the amount of sera neutralization by measuring the reduction in luciferase reporter gene expression following a single round of infection.

### Results

Construction and Design of Primary Isolate HIV-1 Envelopes A panel of plasmids expressing HIV-1 gp160 envelopes from Glade A, B, and C were constructed using the pVAX backbone (Invitogen). All sequences were obtained from GenBank using the accession numbers listed in Table 1. Inserts were RNA and codon optimized to increased expression and cloned into pVAX using either BamHI/XhoI or BamHI/EcoRI. Inserts were isolated from patients that ranged in disease progress from acute/early transmitted isolates to Fiebig stage VI. To confirm the expression of each plasmid, 293T cells were transfected with individual plasmids and flow cytometry was performed using anti-HIV-1 envelope antibody 2G12. Cells were gated on live singles and expression levels were compared to pVax empty vector control. All constructs expressed on the surface of the cells (FIG. 1A and FIG. 1B).

TABLE 1a
Description of inserts used in the study

| Name | Insert | Clade | Tier | Genbank # | Transmission | Stage |
|---|---|---|---|---|---|---|
| A1 | Q769ENVd22 | A | 2 | AF407158 | F-M | acute early |
| A2 | Q168ENVe2 | A | 2 | AF407148 | F-M | acute early |
| A3 | Q842ENVd12 | A | 2 | AF407160 | F-M | acute early |
| A4 | Q461ENVe2 | A | 2 | AF407156 | F-M | acute early |
| A5 | Q23ENV17 | A | 2 | AF004885 | F-M | Fiebig IV |
| A6 | Q259d2.17 | A | 2 | AF407152 | F-M | acute early |
| B1 | WITO4160.33 | B | 2 | AY835451 | F-M | Fiebig II |
| B2 | TRJO4551.58 | B | 3 | AY835450 | M-M | Fiebig II |
| B3 | PVO.4 | B | 3 | AY83544 | M-M | Fiebig III |
| B4 | TRO.11 | B | 2 | AY835445 | M-M | Fiebig III |
| B5 | AC10.0.29 | B | 2 | AY835446 | M-M | Fiebig III |
| B6 | REJO4541.67 | B | 2 | AY835449 | F-M | Fiebig II |
| B7 | RHPA4259.7 | B | 2 | AY835447 | | Fiebig < V |
| B8 | NL43 | B | 1B | AF324493 | | |
| B9 | QHO692.42 | B | 2 | AY835439 | F-M | Fiebig V |
| B10 | CAAN5342.A2 | B | 2 | AY835452 | M-M | |
| C1 | Du123.6 | C | 2 | DQ411850 | FSW | Fiebig VI |
| C2 | ZM53M.PB12 | C | 2 | AY423984 | F-M | |
| C3 | Du422.1 | C | 2 | DQ411854 | FSW | Fiebig V |
| C4 | Cap210.2.00.E8 | C | 2 | DQ435683 | FSW | |
| C5 | Du151.2 | C | 2 | DQ411851 | FSW | Fiebig V |
| C6 | Du156.12 | C | 2 | DQ411852 | FSW | Fiebig < IV |
| C7 | Du172.17 | C | 2 | DQ411853 | FSW | Fiebig VI |
| C8 | Cap45.2.00.G3 | C | 2 | DQ435682 | FSW | |
| C9 | ZM233M.PB6 | C | 2 | DQ388517 | F-M | |
| C10 | ZM249M.PL1 | C | 2 | DQ388514 | F-M | |
| C11 | ZM214M.PL15 | C | 2 | DQ388516 | F-M | |

TABLE 1b
Showing the relationship between the insert and SEQ ID NOs.

| | | Nucleotide | Encoded aa |
|---|---|---|---|
| Name | Insert | SEQ ID NO | SEQ ID NO |
| A1 | Q769ENVd22 | 1 | 2 |
| A2 | Q168ENVe2 | 3 | 4 |
| A3 | Q842ENVd12 | 5 | 6 |
| A4 | Q461ENVe2 | 7 | 8 |
| A5 | Q23ENV17 | 45 | 46 |
| A6 | Q259d2.17 | 9 | 10 |
| B1 | WITO4160.33 | 11 | 12 |
| B2 | TRJO4551.58 | 13 | 14 |
| B3 | PVO.4 | 15 | 16 |
| B4 | TRO.11 | 17 | 18 |
| B5 | AC10.0.29 | 53 | 54 |
| B6 | REJO4541.67 | 19 | 20 |
| B7 | RHPA4259.7 | 21 | 22 |
| B8 | NL43 | 51 | 52 |
| B9 | QHO692.42 | 55 | 56 |
| B10 | CAAN5342.A2 | 57 | 58 |
| C1 | Du123.6 | 23 | 24 |
| C2 | ZM53M.PB12 | 25 | 26 |
| C3 | Du422.1 | 27 | 28 |
| C4 | Cap210.2.00.E8 | 29 | 30 |
| C5 | Du151.2 | 31 | 32 |
| C6 | Du156.12 | 33 | 34 |
| C7 | Du172.17 | 35 | 36 |
| C8 | Cap45.2.00.G3 | 37 | 38 |
| C9 | ZM233M.PB6 | 39 | 40 |
| C10 | ZM249M.PL1 | 41 | 42 |
| C11 | ZM214M.PL15 | 43 | 44 |

Each insert was cloned into the pVAX backbone (Invitrogen) under the control of the cytomegalovirus immediate-early promoter using either BamH1/Xho1 or BamH1/EcoR1. The insert was full length gp160 and was codon optimized to increase protein expression. All sequences were obtained from Genbank using the accession number listed.

### Expression of Plasmids

To confirm the expression of each plasmid, 293T cells were transfected with individual plasmids and fluorescent immunohistochemistry was performed using anti-HIV-1 envelope antibody 2G12. Analysis using gel electrophoresis and staining, showed expression of the encoded protein.

Immunization of a Single Plasmid Expressing Primary Isolate gp160 Produces Limited Binding Titers Against a Consensus gp120 Protein

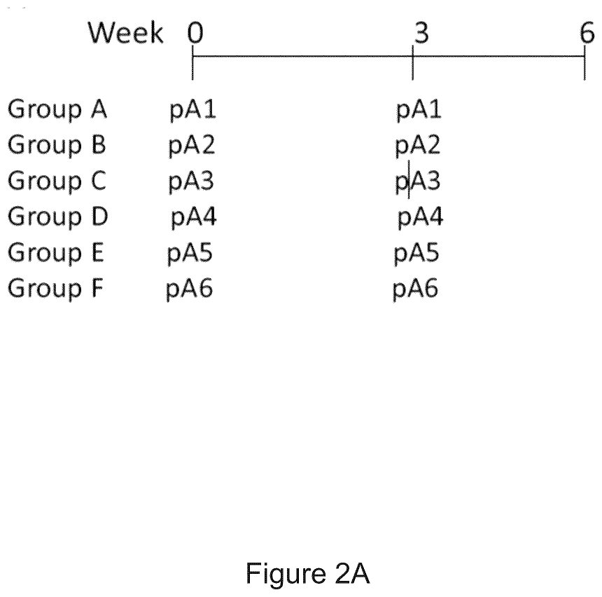

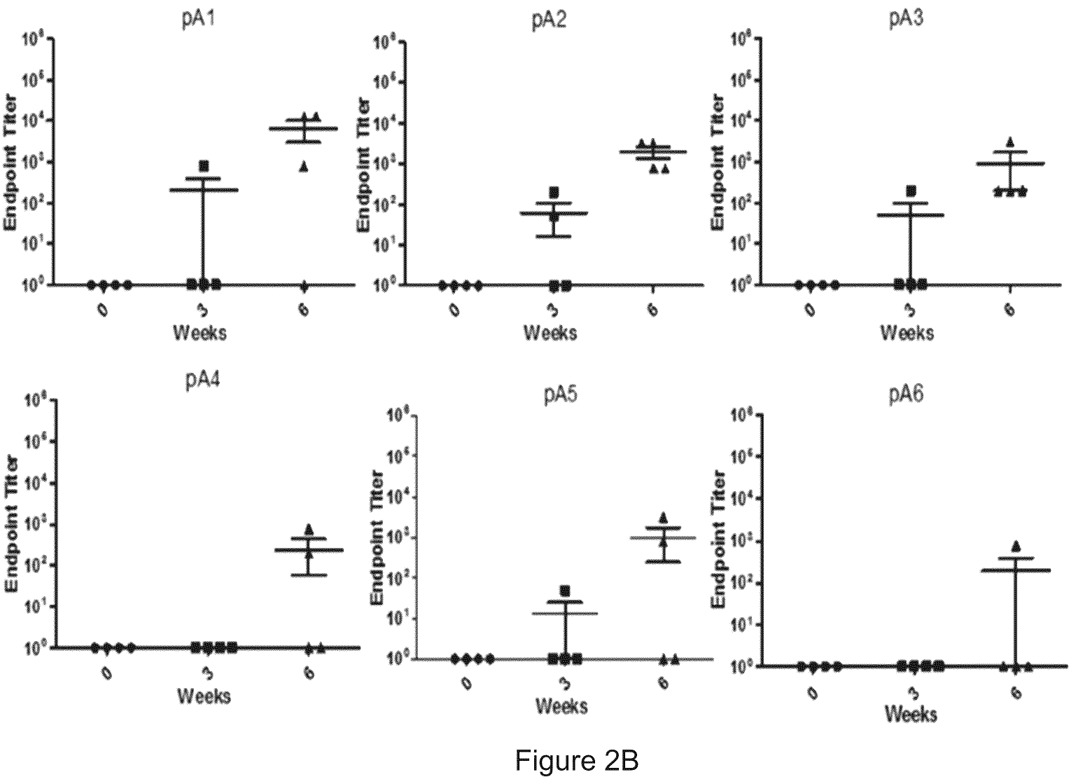

Previous experiments have shown that guinea pigs immunized with plasmids expressing consensus envelope immunogens are able to produce robust binding titers within two immunizations. To determine if plasmids expressing primary isolate envelopes could also induce binding titer responses, groups of four guinea pigs were immunized intradermal with 25 μg of plasmids A1-A6 tri-weekly followed by electroporation (FIG. 2A). Plasmids A1-A5 were able to induce anti-gp120 binding titers after two immunizations (FIG. 2B). However, this response was inconsistent as not all guinea pigs seroconverted after two immunizations. In addition, the level of binding titers was much lower than seen with consensus envelope immunogens.

### Sequential Immunization of Plasmids Do Not Increase Antibody Responses

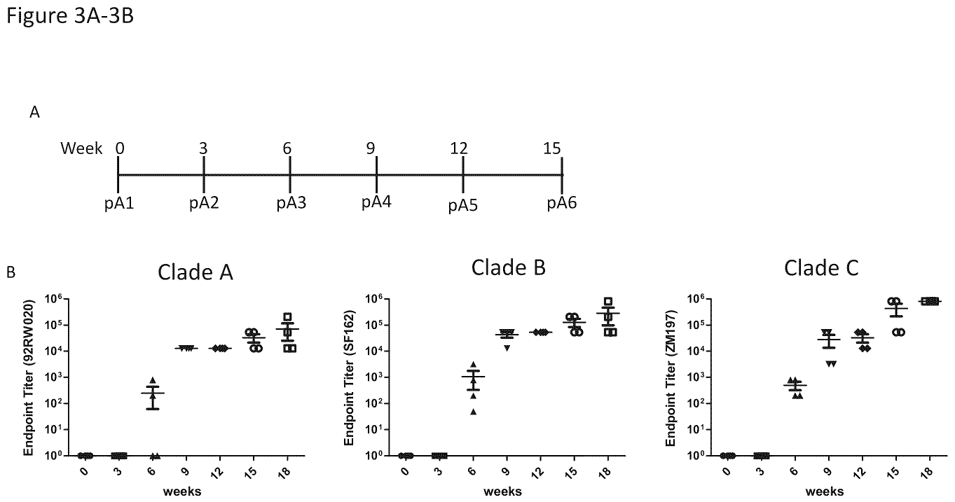

In order to investigate if sequential immunization of plasmids expressing different but related gp160 inserts, four rabbits were immunized with 600 μg of plasmids A1-A6 in a stepwise fashion (FIG. 3A). After three immunization, binding antibodies are detected against primary gp120 envelopes from clades A, B, and C (FIG. 3B). For clades A and B (92RW020 and SF162 respectively), binding titers were only moderately increased after the 4^{th} immunization. However, binding titers to Glade C gp120 (ZM197) continue to increase through the final immunization. Thus, rabbits immunized with multiple different DNA constructs expressing primary gp160 envelopes are able to induce a potent binding humoral response which could induce functional antibodies.

### Formulation of Plasmids Affect the Strength of the Response

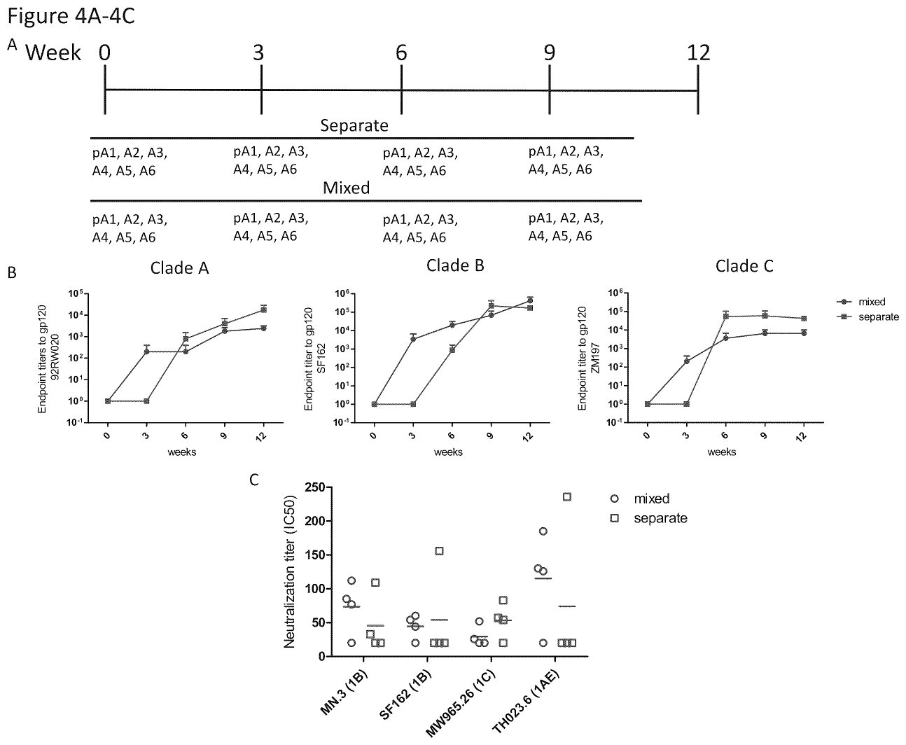

Sequential immunization may be difficult to perform in the field due to different immunizations needed to be given at each visit. Thus we wanted to determine if these same envelopes formulated together in a vaccine could induce a similar humoral responses as seen in the sequential immunization. However, questions arose as to if there would be antigen competition between the groups of envelopes and thus, two vaccinations were performed: one where all of the plasmids were formulated together and another were each plasmid was given in a separate site. Rabbits were immunized four times with 100 μg of each plasmid ID followed by electroporation (FIG. 4A). The total amount of DNA for each immunization was the same across both groups (600 μg total−100 μg/plasmid) and the route and electroporation protocol were the same. The only difference was whether or not the plasmids were immunized separately or mixed together. In both cases, the binding titer response is similar to that induced in the sequential immunizations. Endpoint binding titers to the same primary gp120s were used to determine the induction of humoral responses. Though at the end of the vaccination (week 12) binding titers between the mixed vs separate are similar, the induction of humoral responses is quicker in the mixed group than in the separate group (FIG. 4B). In addition, post final vaccination neutralization titers were slightly, though not significantly, higher in the mix vs separate group for three different tier 1 viruses (MN.3, SF162, and THO23.6) (FIG. 4C). This data suggest that mixing the envelopes together does not dampen the humoral responses but instead, increases the initial seroconversion rate and could induce more superior functional antibody titers. Due to this and the ease mixed formulation provides for vaccine administration, all further studies were performed in this fashion.

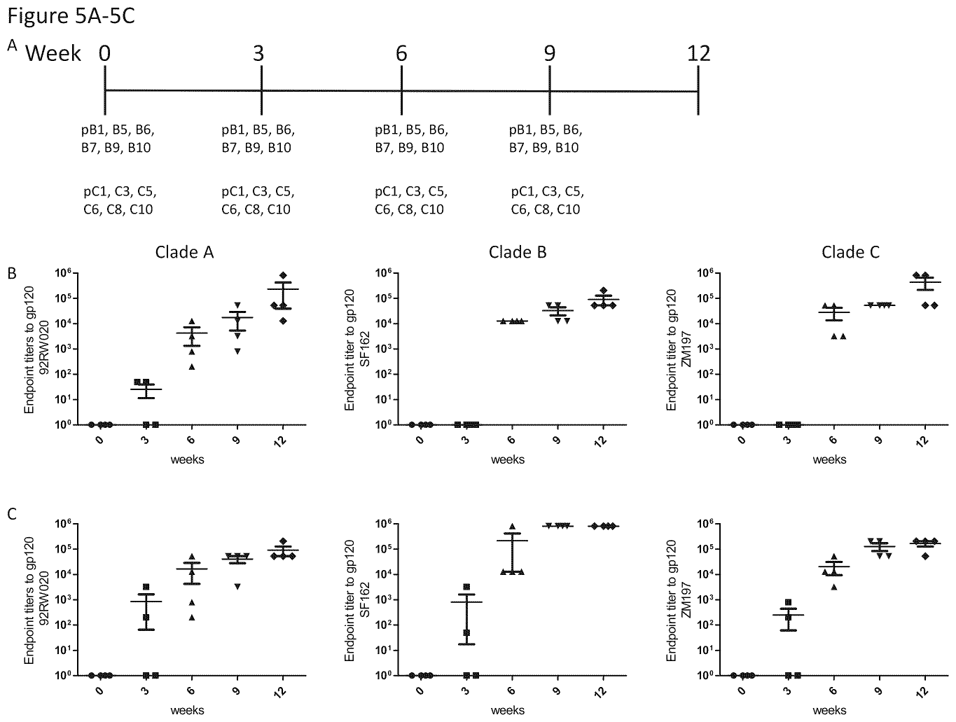

To further investigate the use of small groups of primary envelopes, additional rabbits were immunized with six plasmids expressing either Glade B or Glade C envelopes (FIG. 5A). All envelopes (100 μg/plasmid) were formulated together and delivered to six sites ID followed by electroporation. After two immunizations, half of the animals developed humoral responses in both the Glade B and Glade C regimen (FIG. 5B-FIG. 5C). Even though the animals are immunized with only a single Glade, all rabbits induce strong cross-Glade binding titers which was also seen in the Glade A immunized rabbits. In fact, the Glade C immunized rabbits had the highest binding titer responses to the Glade B (SF162) gp120 protein. Overall, formulating multiple primary transmitter founder or acute envelopes together in a single formulation induces strong cross-Glade binding titers and a limited neutralization profile.

Increasing Diversity within Group Expands Antibody Responses

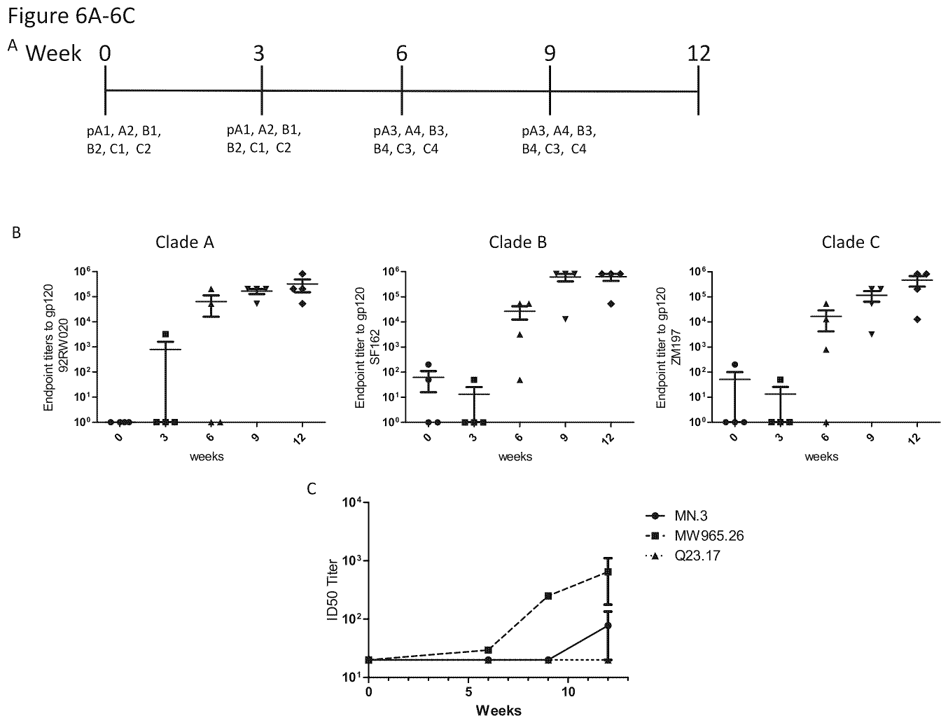

To investigate whether the results seen in the single Glade immunizations could be further expanded upon, two different groups of plasmids were used each containing two Glade A, B, and C primary gp160 envelopes. Four rabbits were immunized with combination 1 (pA1, A2, B1, B2, C1, C2) twice followed by combination 2 (pA3, A4, B3, B4, C3, C4) (FIG. 6A). The plasmids were all formulated together per different combination with 100 μg (600 μg total) of DNA construct used per immunization, delivered ID followed by electroporation. The mean diversity within the groups was 22.0% and 21.0% respectively. The mean diversity intergroup was 20.6%. Once again after two immunization, there is potent induction of binding titers against primary Glade A, B, and C gp120s (FIG. 6B). Neutralization of tier 1 Glade B viruses is induced after immunization of combination 2 and continues to increase after the final immunization at week 9 (FIG. 6C). However, the neutralization profile of sera is still limited in breadth and there is limited neutralization of tier 2 virus in the A3R5.7 cells and no neutralization of tier 2 viruses in the TZM.bl cells (Table 2 group 5). The combination of plasmids expressing two Glade A, B, and C envelope gp160s does appear to induce potent binding titers but limited neutralization breadth.

TABLE 3
Neutralization profile of serum from groups 5, 6, and 7

| Group | Group 5 | | | | Group 6 | | | | Group 7 | | | |
|---|---|---|---|---|---|---|---|---|---|---|---|---|
| Animal | 1 | | 2 | | 1 | | 2 | | 1 | | 2 | |
| Bleed Week | Wk 0 | Wk 12 | Wk 0 | Wk 12 | Wk 0 | Wk 12 | Wk 0 | Wk 12 | Wk 0 | Wk 12 | Wk 0 | Wk 12 |
| MN.3 | <20 | 135 | <20 | <20 | <20 | 226 | <20 | 90 | <20 | 248 | <20 | 524 |
| Tier 1 | | | | | | | | | | | | |
| Clade B | | | | | | | | | | | | |
| MW 965.26 | <20 | 1113 | <20 | 177 | <20 | 530 | <20 | 630 | <20 | 862 | <20 | 287 |
| Tier 1 | | | | | | | | | | | | |
| Clade C | | | | | | | | | | | | |
| Q23.17 | <20 | <20 | <20 | <20 | <20 | 109 | <20 | 39 | <20 | 124 | <20 | 220 |
| Tier 1 | | | | | | | | | | | | |
| Clade A | | | | | | | | | | | | |
| RHPA4258.7 | <20 | <20 | <20 | <20 | <20 | 154 | <20 | 47 | <20 | 214 | <20 | 310 |
| Tier 2 | | | | | | | | | | | | |
| Clade B | | | | | | | | | | | | |
| TRO.11 | <20 | <20 | <20 | <20 | <20 | 36 | <20 | <20 | <20 | 54 | <20 | 57 |
| Tier 2 | | | | | | | | | | | | |
| Clade B | | | | | | | | | | | | |
| Ce1176_A3 | <20 | <20 | <20 | <20 | <20 | <20 | <20 | <20 | <20 | 21 | <20 | 26 |
| Tier 2 | | | | | | | | | | | | |
| Clade C | | | | | | | | | | | | |
| BF1266.431 | <20 | <20 | <20 | <20 | <20 | 143 | <20 | 50 | <20 | 228 | <20 | 364 |
| a Tier 2 | | | | | | | | | | | | |
| Clade C | | | | | | | | | | | | |
| Q842.d12 | <20 | <20 | <20 | <20 | <20 | 288 | <20 | 100 | <20 | 387 | <20 | 716 |
| Tier 2 | | | | | | | | | | | | |
| Clade A | | | | | | | | | | | | |
| C2101.c01 | <20 | <20 | <20 | <20 | <20 | 45 | <20 | <20 | <20 | 84 | <20 | 109 |
| Tier 2 | | | | | | | | | | | | |
| Clade AE | | | | | | | | | | | | |
| RHPA | 23 | 363 | <20 | 435 | 40 | 139 | 31 | 109 | 24 | 404 | 31 | 270 |
| Tier 2 | | | | | | | | | | | | |
| Clade B | | | | | | | | | | | | |
| REJO | <20 | <20 | <20 | <20 | <20 | 438 | <20 | 83 | <20 | 749 | <20 | 329 |
| Tier 2 | | | | | | | | | | | | |
| Clade B | | | | | | | | | | | | |
| CM234-2 | 44 | 34 | 36 | 97 | 62 | 294 | 47 | 110 | 74 | 915 | <20 | 389 |
| Tier 2 | | | | | | | | | | | | |
| Clade AE | | | | | | | | | | | | |

Neutralization was determined using tier 1 and 2 envelopes from clades A, B, C, and AE. In addition, neutralization was determine for a selection of tier 2 isolates in the A3R5.7 cell line.

Creating “Clouds” with Limited Diversity Expand the Neutralization Breadth of Sera

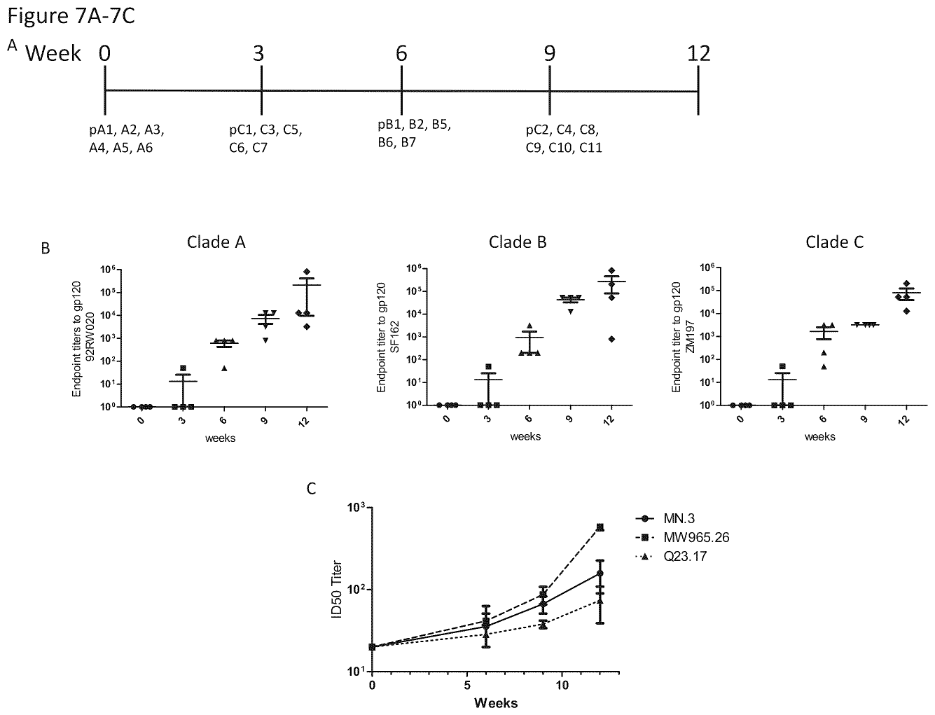

We next wanted to investigate if too much diversity within the vaccinated “cloud” could inhibit responses. Using the same primarily transmitted founder group (pA1-A6) as a priming dose, four rabbits were immunized with additional “clouds” or groups of plasmid which were more limited in diversity and stayed within clades (FIG. 7). The intra-cloud diversity ranged from 12.4-16.4% and inter-cloud was consistently around 20%. Each immunization was between 500 μg -600 μg of total DNA (100 μg of each plasmid) mixed together and administered ID to five or six separate sites followed by electroporation. Using this limited intra-cloud diversity regiment did not disrupt the ability to induce potent cross-Glade binding tiers against the three primary isolate gp120 (FIG. 7B). There is a consistent boosting of titers after every immunization with the highest binding titers obtained after the final immunization at week 9. Although at a low level, as early as week 6 (post two immunizations), sera is able to neutralize tier 1 viruses from clades A, B, and C (FIG. 7C). This neutralization ability continues to rise after every immunization with final IC50 titers as high as 1/630 (Table 2 group 6). The limited diversity cloud vaccination is able to induce a more potent neutralization profile as sera is able to neutralize tier 2 viruses in A3R5.7 cells and even low but consentient neutralization of tier 2 virus in TZM.bl cells for the two rabbits tested. The ability to induce this robust of a response by DNA alone has yet to be seen and could lend itself well to further expansion by boosting with a different platform.

Highest Induction of Robust Antibody Responses in Rabbits Primed Twice with Transmitted Founder “Cloud”

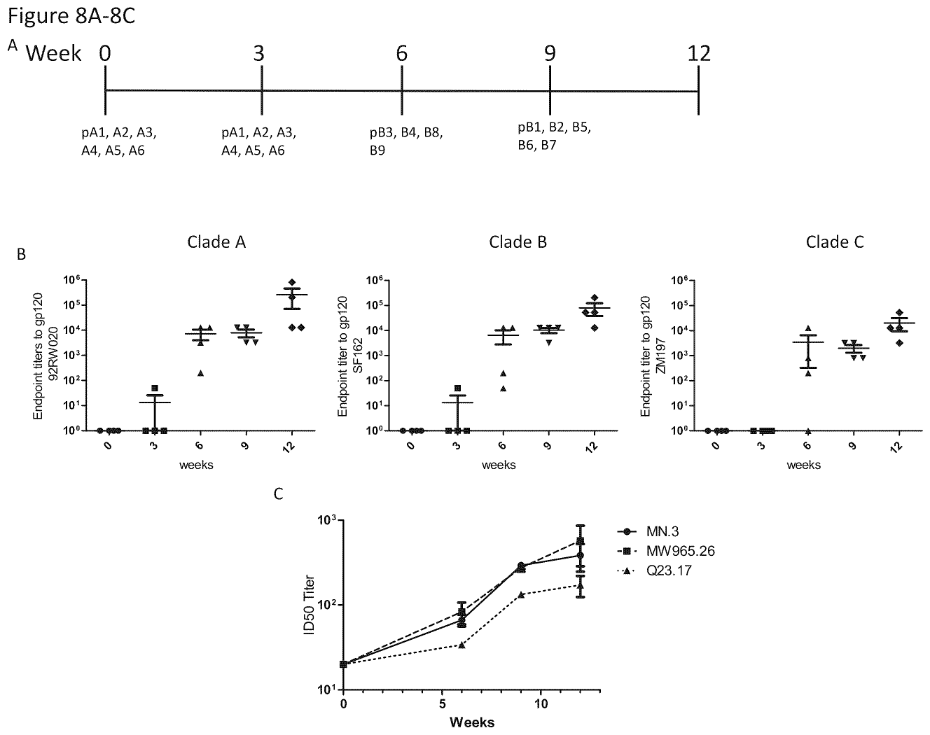

The final group of rabbits looked to determine if these responses would increase by priming with the same group twice. This would allow for the immune system to potentially honing in on specific epitopes which would later be expanded by boosting with additional clouds. Rabbits were immunized twice with the transmitted founder plasmid cloud and boosted with primarily Glade B immunogens (FIG. 8A). The intra-cloud diversity ranged from 13.3-14.3% and the inter-cloud diversity between 14-17.6%. Thus this regiment has the lowest diversity between the clouds compared to the other two combinations. This low intra-cloud diversity does not limit the responses as potent binding titers are induced in all animals after 3 immunizations (FIG. 8B). The highest and quickest induction of neutralization is seen for this group, with the most powerful response happening after the final immunization (FIG. 8C). In addition, sera from two rabbits were able to neutralize more isolates at higher IC50 concentrations than groups 4 and 5 (Table 2 group 7). This includes hard to neutralize tier 2 viruses where only one virus (Ce1176_A3) is not able to be neutralized. This neutralization capacity holds against Glade C and Glade AE viruses, both of which the rabbits never saw any isolates from either Glade. Thus, priming rabbits with two immunizations of plasmids expressing primarily transmitted founder immunogens seems to focus the immune system in a way that allows for effective induction of broadly binding and neutralizing antibodies.

Non-Human Primates Immunized with “Clouds” of Primary Envelopes Induce Potent Cellular and Humoral Responses

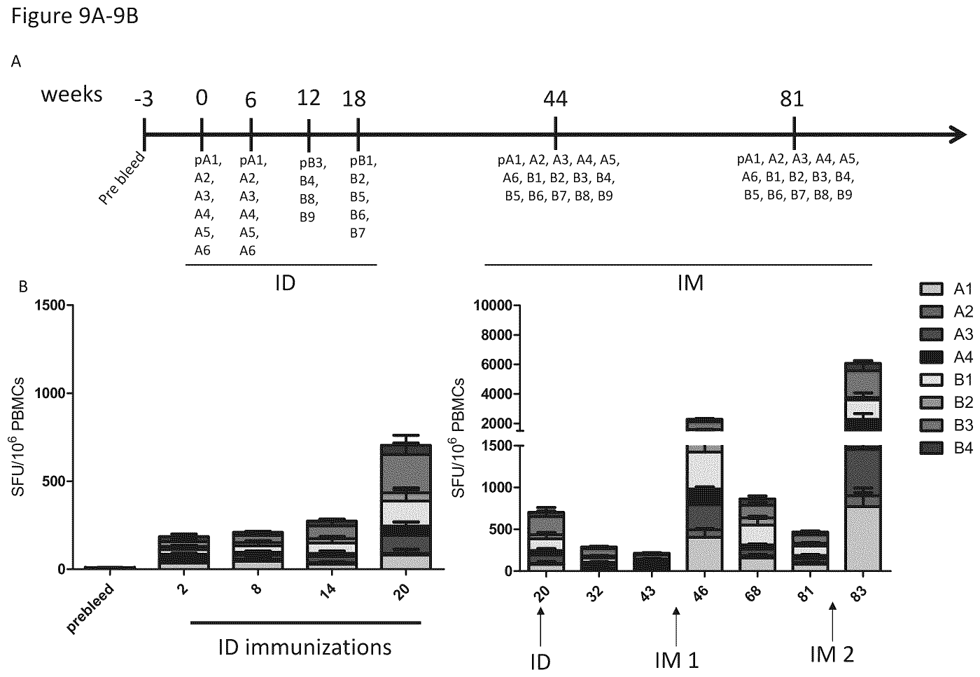

To further characterize the vaccine induced responses produced by the most potent regiment (FIG. 8A), eight rhesus macaques (RhMs) were immunized with a similar vaccine regiment. On weeks 0, 6, 12 and 18, the NHP received a mixture of different envelopes (1 mg/plasmid) formulated together and delivered ID followed by electroporation (FIG. 9A). To further expand the vaccine induced responses, at weeks 44 and 81 post first vaccination, all animals received all of the envelopes from vaccination 1-4 (1 mg/plasmid) delivered IM at a single site followed by electroporation. Cellular and humoral responses were followed two weeks after each vaccination. After only a single immunization, IFN-γ spot forming units (SFU) are detected against consensus clades A and B peptides (FIG. 9B). These responses are not boosted with the second immunization of the priming cloud but are expanded upon after the third and fourth immunization. After the final ID immunization, the average total IFN-γ SFU is around 800. Though there is contraction into the memory phase, cellular responses can still be detected against consensus Glade A and B almost 6 months after final ID immunization. After the first IM boosting immunization at week 44, cellular responses expand greatly to levels over double the amount seen after final ID immunization. Over eight months after IM immunization, cellular responses have contracted but remain around the levels seen after final ID immunization. Upon second IM boost, cellular responses again expand above those seen after the previous IM immunization with IFN-γ SFU averaging around 7000. These responses are extremely high, especially since they are against unmatched peptides. In addition, since consensus peptides are used, this suggest that these small “clouds” of immunogens are able to induce potent cellular responses against conserved regions within the envelope. This could be important for the induction of cytotoxic T cells against envelope as well as providing broad CD4 T cell help.

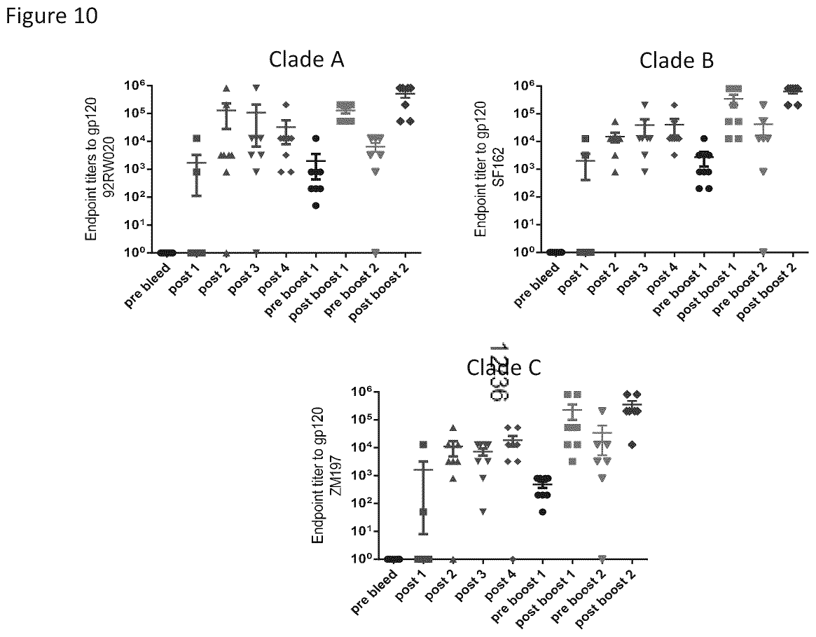

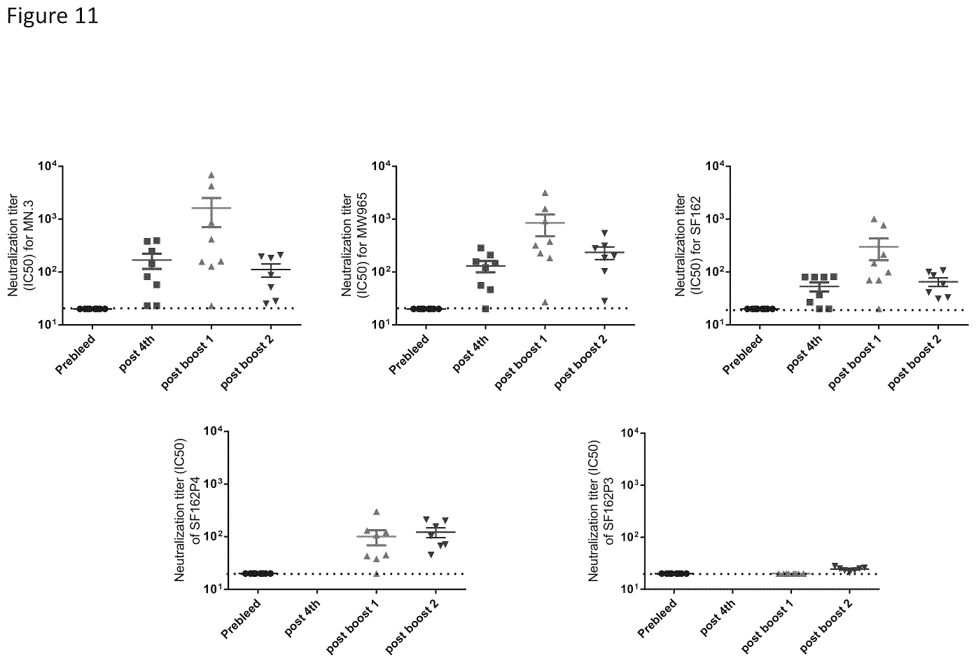

The primary envelope cloud immunization also induces potent humoral responses. After a single immunization, two out of eight RhMs seroconvert to Glade A, B and C primary gp120 proteins. After the final ID immunization, all animals have strong endpoint binding titers against the primary envelopes averaging above 10⁴ (FIG. 10). These responses also contract down in the memory phase but remain high (average above 10³) six month post last ID immunization. Similar to cellular responses, after the IM boost, binding titers reach levels higher than after ID immunization with the average binding titer above 10⁵. These responses are also slightly boosted after a second IM immunization to levels reaching 10⁶ binding titers. In addition to binding titers, the vaccination regimen also induces functional antibodies. Using only DNA vaccination we are able to get cross Glade neutralization titers against a diversity of tier 1 viruses (FIG. 11). After ID immunization, neutralization titers for MN.3, MW965 and SF162 average above or around 10². After the first IM boost, levels are increased to above 10³ for MN.3 and MW965 and just below 10³ for SF162. Additionally after the first IM boost, neutralization titers are detected against infectious molecular clone (IMC) of SF162P4 virus. These average above 10². After the second IM boost we do not see levels increase above those observed after the initial IM boost. In fact, for MN.3, MW965 and SF162, the levels were lower and usually averaged around the same titers as those seen after the ID immunizations. However, levels against SF162P4 IMC were maintained and importantly, there were limited but low neutralization titers induced against the tier 2 virus SF163P3. These data supports the use of primary transmitter founder envelopes deliver in small “cloud” immunizations for the induction of potent cellular and humoral responses.

### The Mixed Clouds Induce Primarily 173 Binding Antibodies

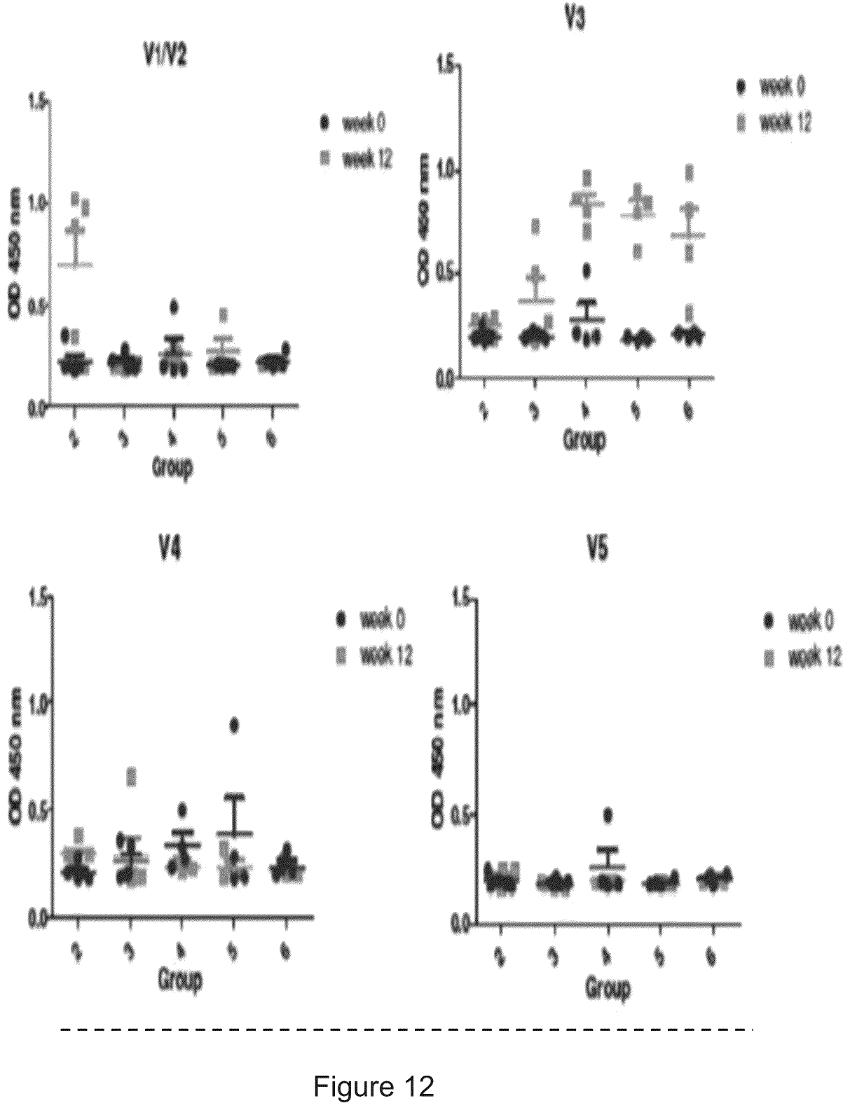

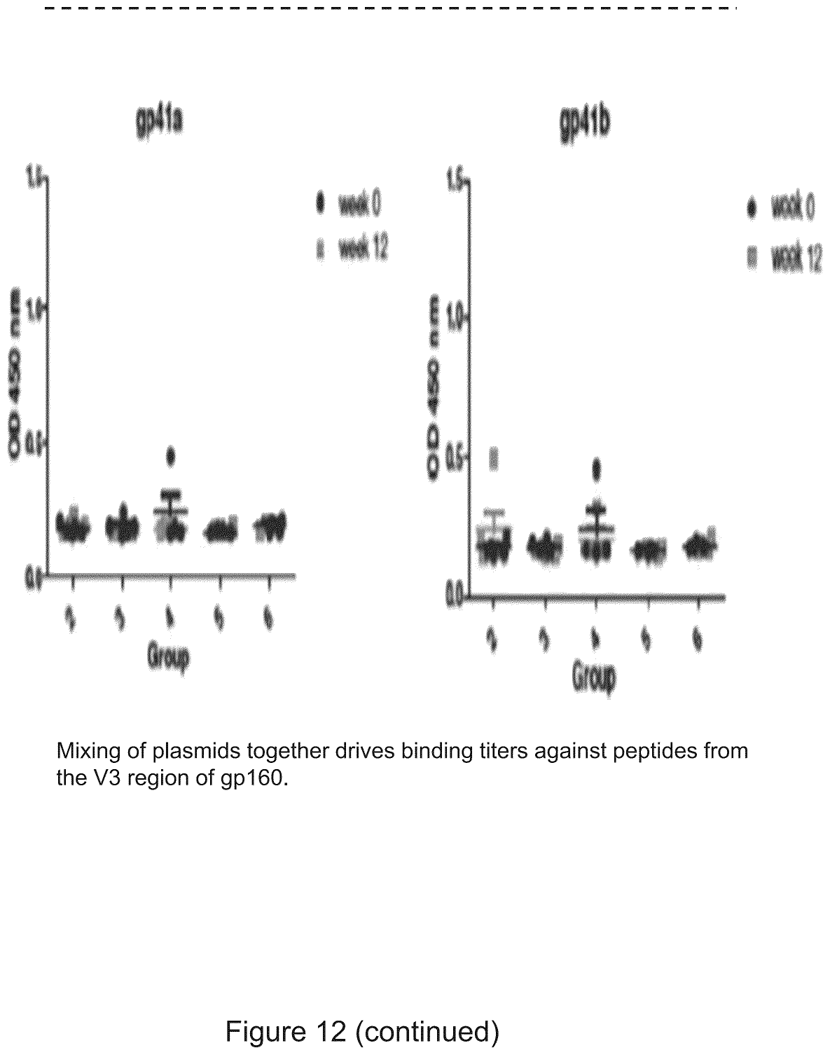

In order to determine the binding epitope of sera antibodies, linear 15mer peptides will 11 amino acid overlap consisting of the entire consensus Glade C gp160 (NIH AIDS Reagents and Reference program) were used to create pools of variable regions of gp120 as well as two pools for gp41. Binding ELISAs were performed using each pool and sera from week 0 and week 12 for the groups which induced binding titers (groups 2-6). All groups except for group 2 induced a high amount of binding to the V3 peptide pool (FIG. 12). Group 2 which consisted of the same DNA as group 3 but each plasmid was immunized to a separate site seemed to drive binding titers to the V1/V2 pool. Both the V1/V2 and the V3 have classes of broadly neutralizing antibodies associated with them (PG and the PGT family respectfully) (reference). However, this binding epitope analysis was not expansive as it did not cover any of the constant regions and relied on linear epitopes. Many potent broadly neutralizing antibodies, including the PG's and the CD4 bs antibodies rely on conformational or quaternary epitope binding. Thus additional test should be performed to determine the exact epitope the vaccination is able to induce.

### Example 2—Extreme Polyvalency Induces Potent Cross-Glade Cellular and Humoral Responses in Rabbits and Non-Human Primates

As described herein, over 40 different DNA plasmids have been developed which express consensus as well as primary HIV Envs. All of these optimized plasmids are able to induce both cellular and humoral responses in mice. Different combinations of Envs were tested in rabbits to further characterize the humoral responses and explore neutralization. Rabbits immunized with clusters of Glade A transmitted founder (TF) gp160 DNA induced cross-Glade binding titers with limited neutralization. Including TF Envs from different clades increased binding titers as well as neutralization breadth and potency. Formulating the gp160s to be administered to the same site induced faster seroconversion than delivering the Envs at separate sites. The most potent combination was moved forward into non-human primates, which were immunized with clusters of gp160 DNAs (14 different Envs in total) at weeks 0, 4, 8, 12 and boosted at weeks 48 and 85. The vaccine induced cross-Glade cellular and humoral responses after two immunizations. These responses increased after each immunization and were maintained into memory. In addition to binding, the vaccine also induced tier 1A and 1B neutralization titers and antibody dependent cellular cytotoxicity against both homologous and heterologous targets. Boosting at week 48 and 85 further increased both responses.

It is shown herein that DNA plasmids encoding consensus and TF Envs are expressed and induce a potent immune response. It is observed herein for the first time that exposure of the immune system to multiple Envs at one time can dramatically change the immune phenotype by inducing broader breadth of responses which has significant implications for HIV vaccine development.

### Methods

### Envelope Immunogens

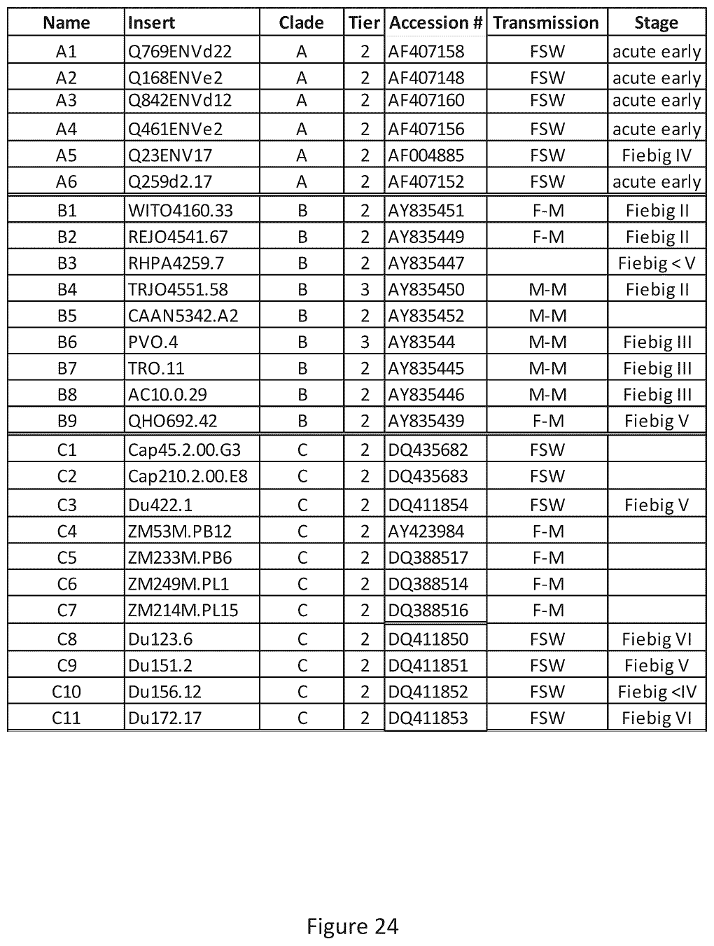

Plasmids expressing codon and RNA optimized HIV Envelope glycoproteins (gp160) were made synthetically using OptimumGene® Codon optimization analysis (GenScript, Piscataway, NJ). Inserts were then cloned into the pVAX (Invitrogen, Carlsbad, CA) backbone using either BamHI/XhoI or BamHI/EcoRI cloning sites. Each insert was under the control of the cytomegalovirus immediate-early promoter. A description of each of the inserts can be found in FIG. 24.

### Expression of Plasmids

Each plasmid was tested in vitro for proper expression. Briefly, HEK 293T cells (ATCC, Manassas, VA) were cultured in Dulbecco's Modified Eagle Medium (Thermo Fisher Scientific, Carlsbad, CA) supplemented with 10% fetal bovine serum (Atlas, Ft. Collins, CO) and 1% penicillin and streptomycin (Thermo Fisher Scientific). Twenty four hours before transfection, 7.5×10⁵ cells were plated in 1.5 mls of media in a 6 well dish. Each plasmid was used in a separate transfection with pVax empty backbone serving as a negative control. Transfection was performed using NeoFectin transfection reagent (NeoScientific, Cambridge, MA) following manufactures protocol. Forty-eight hours after transfection, cells were collected and washed with PBS and lysed using Cell Signaling lysis buffer (Cell Signaling, Danvers, MA) modified with EDTA-free protease inhibitor (Roche, Basel, Switzerland). Bradford assay was used to quantify protein concentration of lysate following manufactures protocol (BioRad, Hercules, CA). Normalized lysate was then run on a NuPAGE® 12% Tris-Acetate gel and transferred to a PVDF membrane following manufactures protocol (Thermo Fisher Scientific). After 1 hour blocking with LI-COR Odyssey blocking buffer (LI-COR, Lincoln, Nebraska), membranes were probed overnight with a 1:1000 dilution of human 2G12 antibody (ImmuneTechnologies Corp, New York, NY) and 1:5000 dilution of mouse-anti human β-actin (Sigma Aldrich, St. Louis, MO) as a loading control. After washing with PBS-Tween, 1:10,000 dilution of secondary goat anti-human IRdye 680 and goat anti-mouse IRdye 800CW (LI-COR) antibodies were added in blocking buffer supplemented with 0.1% Tween and 0.01% SDS (Sigma Aldrich). Membranes were probed for 1 hour at room temperature followed by washing with PBS-Tween and PBS. Membranes were then scanned using LI-COR Odyssey CXL.

### Immunization of Mice

To test for immunogenicity, 6-8 week old C57Bl/6 mice (Jackson Laboratories, Bar Harbor ME) were immunized with 25 μg of each plasmid followed by in vivo electroporation (EP) using the CELLECTA® 3P adaptive constant current electroporation device (Inovio Pharmaceuticals, Plymouth Meeting, PA) as previously described (Muthumani et al., 2013, PLoS One 8:e84234). Mice were immunized 3 times at 2 week intervals and sacrificed one week after final vaccination to assess vaccine induced immune responses.

### Immunization of Guinea Pigs for Formulation Study

Female Hartley guinea pigs (300-350 grams) were immunized with 100 μg of DNA intradermal mantoux injection every 3 weeks with in vivo EP as described above. Six Glade A plasmids were delivered to six separate sites or formulated together and spread across six different sites. Each guinea pig received the same total amount of DNA, volume of injection and sites of immunization. Blood was collected for analysis before every vaccination.

### Immunization of Guinea Pigs for In Vivo Analysis

In order to differentiate each of the Envelopes, three tags were added via plasmid mutagenesis (Genscript): pQ168ENVe2-his, pQ23ENV17-flag, pDu151.2-cMyc. All tags were added to the C-terminus of the protein. Two female Hartley guinea pigs (300-350 grams) were injected with 16.5 μg of each plasmid (50 μg of total DNA) formulated together and injected ID using a mantoux injection. The area was then immediately electroporated using the ELGEN-SEP 4×4 array (3 pulses at 25V, pulse length 100 msec, pulse delay 200 msec). Guinea pigs were then euthanized 24 hours after treatment and the vaccinated skin was harvested. The skin biopsies were fixed by immersion in 4% paraformaldehyde (Sigma Aldrich) for 12 hr at 4° C. After washing with PBS, biopsies were immersed in 15% sucrose solution followed by immersion in 30% sucrose. The biopsies were then embedded in O.C.T compound (Fisher Scientific) and snap frozen. The skin was then sectioned in cryostat at a thickness of 15 μm, placed on a glass slide and stored at −80° C. Sections were then incubated with BSA-Histology buffer (0.5% (v/v) Triton X, 3% (w/v) BSA in 1×PBS) for 30 min at room temp. Primary antibodies were then added to each section and incubated for 2 hours at room temp. Primary antibodies include: Goat anti-FLAG (1:1000 QED Bioscience, San Diego, CA); mouse anti-HIS (1:200 Abcam, Cambridge, UK) and rabbit anti-myc (1:100, Abcam). After washing with PBS, the first round of secondary antibodies were added in BSA-Histology buffer. Following washing with PBS, sections were incubated with a second round of secondary antibodies. Round one included: donkey anti goat IgG-AF488 (1:200 Abcam) and donkey anti-rabbit IgG-AF55 (1:200 LifeTechnologies). The second round included goat anti-mouse-AF647 (1:200 Invitrogen). Sections were washed again and mounted with DAPI-Fluoromount (Fisher Scientific) and covered with a coverslip. Sections were imaged with Olympus BX51 Fluorescent Microscope, QImaging Retiga3000 camera and QImaging software.

### Immunization of Rabbits

Female New Zealand white rabbits (1900 grams) were immunized using 100 μg/plasmid of DNA intradermal every 3 weeks with in vivo EP as described above. All plasmids were formulated together and injected into multiple sites (3-6 depending on the number of plasmids). Each site received 100 μg of mixed DNA in a 100 μl mantoux injection. Blood was collected for analysis before every vaccination.

### Immunization of Non-Human Primates

Four Indian rhesus macaques received six vaccinations: the first four were administered intradermally and the last two were administered intramuscularly. The first and second vaccination on weeks 0 and 6 were a combination of six Glade A primary Envelopes (1.0 mgs each), formulated together and delivered to 6 separate sites. The third immunization delivered on week 12 was a combination of three Glade B Envelopes (1.0 mgs each), formulated together and administered to three different sites. The four immunization delivered on week 18 was a combination of five Glade B Envelopes (1.0 mgs each), formulated together and administered to five different sites. The fifth and six vaccination were given on weeks 44 and 81, composed of all 14 Envelopes (1.0 mgs each) formulated together and delivered to a single site. All DNA deliveries were followed by in vivo EP with the constant current CELLECTRA® device (Inovio Pharmaceuticals, Plymouth Meeting, PA) with 3 pulses at 0.5A constant current, a 52 ms pulse length and 1s rest between pulses.

### Blood Collection

Animals were bled 2 weeks following each immunization (weeks 2, 8, 14, 20, 46, 83) and at memory time points (weeks 32, 43, 68, 81). Blood (15 ml at each time point) was collected in EDTA tubes and peripheral blood mononuclear cells (PBMCs) were isolated using standard Ficoll-Hypaque procedure with Accuspin tubes (Sigma-Aldrich). An additional 10 ml was collected into clot tubes for serum collection.

### Mouse IFN-Gamma Enzyme-Linked Immunospot Assay (ELISpot)

Ninety-six well filter plates (Millipore, Billerica, MA) were coated with anti-IFN-γ capture antibody (R&D, Minneapolis, MN) overnight at 4° C. Spleens were isolated from mice one week after final immunization. After processing the spleens as previously described (Muthumani et al., 2013, PLoS One 8:e84234), 2×10⁵ cells were added to the blocked plates. Cells were stimulated with overlapping 15mer peptide pools for consensus Glade A, B, or C gp160 (5 μg/ml per peptide). Media alone and concacavalin A (Sigma Aldrich) were used as negative and positive controls respectively. After 18 hrs of stimulation, the plates were washed and secondary detection antibody (R&D) was added for 24 hrs at 4° C. Plates were then washed and developed using the ELISpot Blue Color Module (Millipore) per the manufactures protocol. Plater were then scanned and counted using CTL-ImmunoSpot® S6 FluoroSpot plate reader (CTL, Shaker Heights, OH).

### Mouse Serum Binding Using Enzyme Linked Immunosorbent Assay (ELISA)

Before sacrificing, serum from mice was collected to determine the vaccine induced humoral responses. Maxisorp 96 well plates (Thermo Fisher Scientific) were coated with 1 μg/ml of consensus Glade A, B, or C gp120; consensus Glade A, B, or C gp140; or HXBC2 gp41 (Glade B) (Immune Technology Corp.) in PBS and stored at 4° C. overnight. After blocking with 10% fetal bovine serum (FBS) in PBS for 1 hour, mouse serum was diluted 1:50 in 1% FBS in PBST (0.1% Tween). After 1 hour at room temperature and washing, secondary goat anti-mouse HRP-labeled antibody (Santa Cruz Biotechnology, Dallas, TX) was used at a 1:5000 dilution. Plates were washed and developed for 5 minutes using SimgaFast OPD tablets (Sigma Aldrich) and stopped with 100 μl of 2N sulfuric acid (Sigma Aldrich). The OD450 nm was determined using the Promega GloMax plate reader (Promega, Madison, WI).

### Endpoint Binding Titer ELISA

Maxisorp 96 well plates (Thermo Fisher Scientific) were coated with 1 μg/ml of 92RW020, SF162, or ZM197M (Immune Technology Corp) and incubated overnight at 4° C. Plates were blocked as described above for 1 hour at room temperature. Plates were then washed again and incubated with specific guinea pig, rabbit or NHP sera diluted with 1% FBS in 1×PBS+0.02% Tween-20 for 1 hour at room temperature. Dilutions started at 1:50 and then a four-fold dilution was performed. After washing, plates were incubated with dilutions of horseradish peroxidase-conjμgated goat anti-guinea pig (1:2000) or donkey anti-rabbit (1:5000) IgG (Santa Cruz Biotech) or goat anti-NHP (1:5000) (Southern Biotech, Birmingham, AL) for 1 hour at room temperature. The plates were developed and read as described above. Endpoint titers were determined as previously reported (Frey et al 1998). Briefly, the upper prediction limit of Envelope specific IgG antibodies was calculated using the Student t distribution. The upper prediction limit was defined as the standard deviation multiplied by a factor based on the number of naive controls and a 95% confidence interval. Endpoint titer was the lowest dilution that remained above the upper prediction limit.

### Avidity Index ELISA

Plates were coated with 1 μg/ml of either 92RW020 (clade A), Sf162 (clade B) and ZM197 (clade C) gp120 (Immune Technology, New York, NY) in PBS. After blocking, guinea pig or NHP serum was diluted 1:100 or 1:500 (respectively) in 1% FBS in PBS-T. Each sample was run in quadruplicate where half of the wells were treated and half were untreated. After 1 hour incubation, plates were washed 5 times with PBS-T. Half of the wells for each sample were incubated with denaturing reagent, 8M urea, for 5 minutes while the others were incubated with PBS. Plates were washed and incubated with goat anti-guinea pig IgG HRP (1:2000) (Sana Cruz Biotech) or mouse anti-NHP IgG HRP (1:5000) (Southern Biotech, Birmingham, AL) in 1% FBS in PBS-T. Plates were then developed as described above and OD450 values were obtained. The avidity index was determined by dividing the OD450 values of the treated by the untreated and multiplying by 100.

### Neutralization

Neutralization was determined using the previously described TZM-bl based assay (Seaman et al., 2010, J Virol 84:1439-52). The 50% inhibitory dose (ID₅₀) titer was determined as the serum dilution that caused a 50% reduction in the RLU compared to the level in the virus control after subtraction of the cell control background.

### Rhesus IFN-Gamma ELISpot

To determine cellular responses, interferon-gamma (IFN-γ) ELISpots (MabTech, Stockholm Sweden) were performed following manufactures protocols. Isolated PBMCs were stimulated overnight in the presence of either specific peptide antigens (Consensus Glade A and B Envelope peptides (NIH AIDS Research & Reagent Program, Germantown, MD), R10 (negative control), or anti-CD3 (positive control). All samples were run in triplicate. Spot-forming units were determined using the CTL-ImmunoSpot® S6 FluoroSpot plate reader.

### Intracellular Staining of PBMCs

Intracellular staining of PBMCs was performed as previously described (Hutnick et al., 2012, Hum Gene Ther 23:943-50). Briefly, after isolation, PBMCs (1-2×10⁶) were stimulated with pools of either consensus Glade A, B or C peptides for 6 hours in a 96 well U-bottom plate. Each peptide pool contained approximately 1 μg of each peptide. Media only (R10) and PMA (0.1 μg/ml) and ionomycin (0.5 μg/ml) (BD Bioscience, San Jose, CA) were used as negative and positive controls respectively. All stimulations were performed in the presence of Golgi stop/Golgi plμg™ (1:500 dilution BD Biosciences) and anti-CD107a (PE cy7 clone H4A3 BD Bioscience). After stimulation, cells were washed with PBS and stained with violet amine-reactive dye Live/Dead stain (Life Technologies, Carlsbad, CA) for 5 minutes followed by surface staining for 30 minutes at room temperature. Surface stain included CD4 (PECy5.5 clone S3.5 Invitrogen), CD8 (BV650 clone SK1 Biolegend, San Diego), CD95 (PE cy 5 clone DX2, Biolegend), CD28 (BV510 clone CD28.2 Biolegend) and dump channel antibodies CD14 (Pacific Blue clone M5E2 Biolegend) and CD16 (Pacific Blue clone 3G8 Biolegend). Cells were washed with PBS and fixed/permeabilized with BD Cytofix/Cytoperm (BD Biosciences) for 15 minutes at room temperature. Following washing with BD Perm/Wash buffer, cells were stained with intracellular antibodies for 1 hour at room temperature. Intracellular stain included CD3 (APC-Cy7, clone SP34-2 BD Bioscience), IL-2 (PE clonse Mq1-17H12, Biolegend), IFN-γ (APC, clone B27 Biolegend), and TNF-α (PE-Cy7 clone Mab11, Biolegend). Cells were analyzed using a modified BD LSR II (BD Biosciences) and analysis performed with FlowJo 9.2 (Tree Star, Ashland, OR).

### Binding Antibody Multiplex Assay (BAMA)

To further determine binding to various gp120s, gp140s and V1/V2 scaffold proteins, a customized multiplex binding assay was used as previously described (Tomaras et al., 2008, J Virol 82:12449-63; Haynes et al., 2012, NEJM 366:1275-86). Serum from week 20 (post ID), week 46 (post IM 1) and week 83 (post IM 2) were tested at six 5-fold serial dilutions starting at 1:80. Area under the curve (AUC) was calculated using GraphPad Prism.

### Antibody Dependent Cellular Cytotoxicity (ADCC)

ADCC activity against various Env coated target cells was measured using the ADCC-GranToxiLux (GTL) assay as previously described (Pollara et al., Cytometry A 79:603-12).

Briefly, target cells were CEM.NKR_{CCR5} cells (NIH AIDS Reagent Program, Division of AIDS, NIAID, NIH: CEM.NKR-CCR5) coated with recombinant HIV gp120 against WITO (B), JR-FL (B) and 92MG037.1 (A) or gp140 1086 (C). Effector cells were PBMC isolated from a HIV seronegative human donor heterozygous for 158F/V polymorphic variants of Fcγ receptor 3A. NHP serum was tested at baseline, week 20 (2 weeks post 4^{th} ID immunization), week 46 (2 week post 1^{st} IM boost), and week 83 (2 weeks post 2^{nd} IM boost). Serum samples were tested using 4-fold serial dilutions ranging from 1:100 to 1:102,400. ADCC titers were calculated as the dilution at which responses were greater than or equal to 8% GzB expression.

### Statistics

Statistical analysis was performed using GraphPad Prism (GraphPad Software, Inc. La Jolla, CA). Analysis among groups was performed using an independent T-test and a Mann-Whitney test depending on normalcy of data when two groups were being compared and an ANOVA when three groups were being compared. A p-value less than 0.05 was considered statistically significant.

### Results

### Construction and Design of Primary Isolate HIV Envelopes and In Vitro Expression

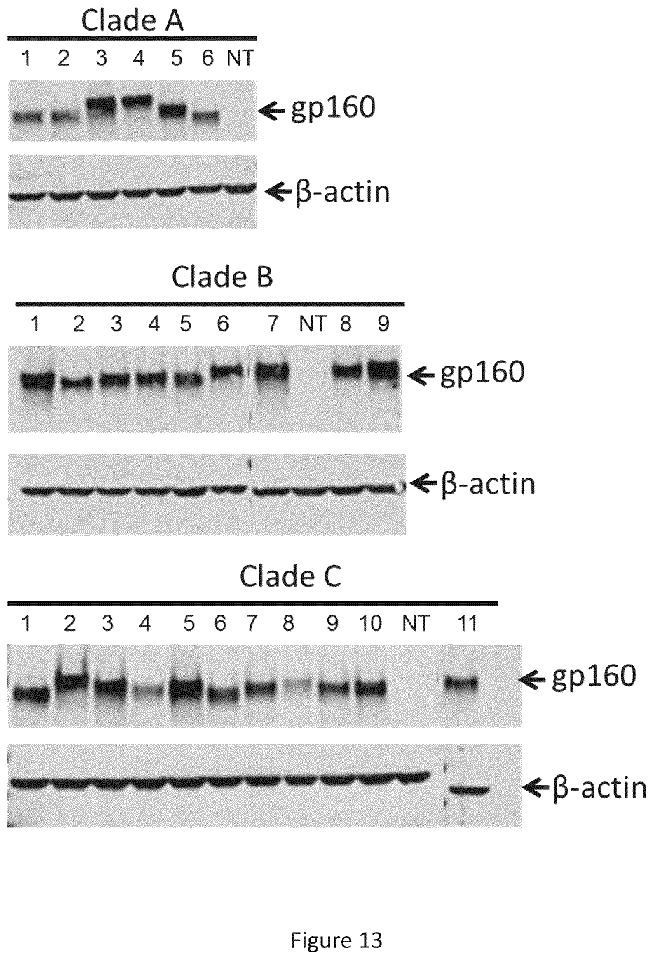

A panel of plasmids expressing RNA and codon optimized HIV gp160 primary Envelopes from Glade A, B, and C were constructed using the pVAX backbone. All sequences were obtained from GenBank using the accession numbers listed in FIG. 24. Envelope sequences were isolated from patents that ranged in disease progress from acute/early transmitted isolates to Fiebig stage VI (Li et al., 2006, J Virol 89:11776-90; Li et al., 2006, J Virol 79:10108-25; Wilen et al., 2011, J Virol 85:8514-27). To confirm expression of each plasmid, western blot analysis was performed on transfected 293T lysate. All plasmids expressed and were detected by the neutralizing antibody 2G12 (FIG. 13).

### Immunogenicity of Primary HIV Env Plasmids in Mice

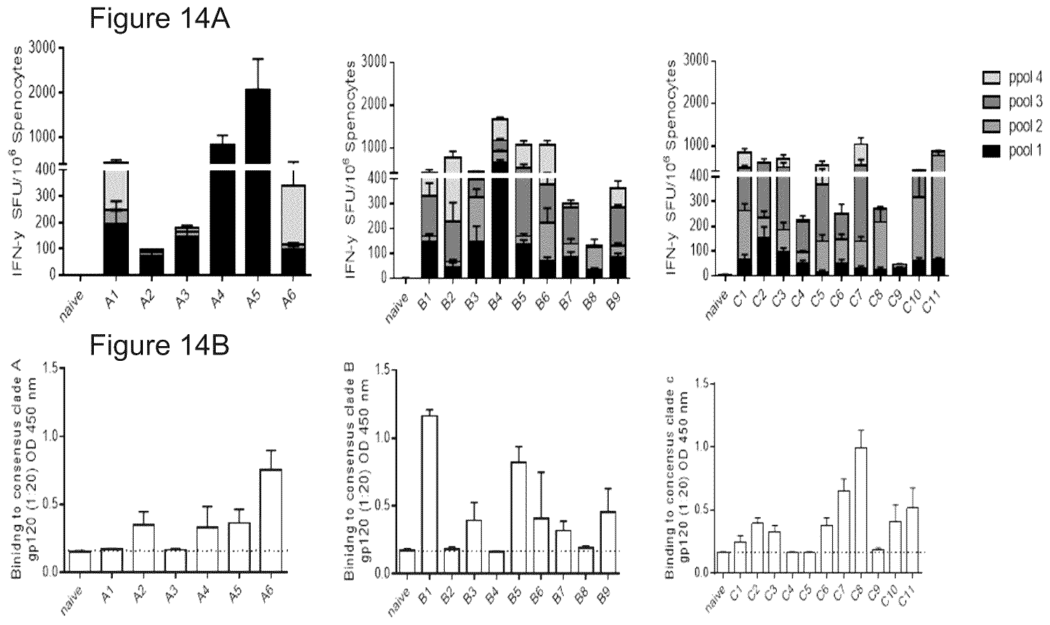

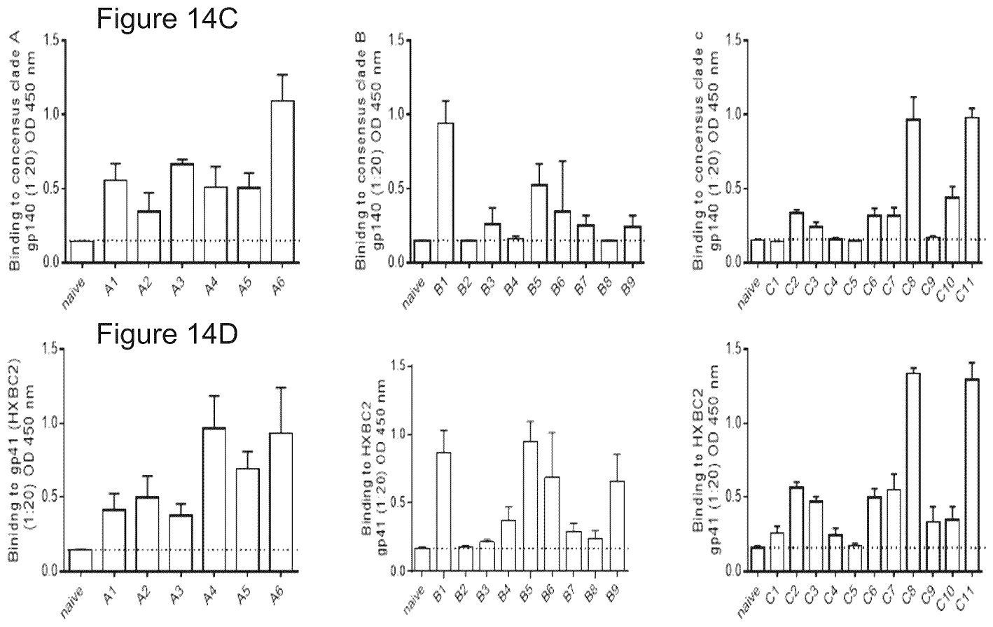

To ensure that each plasmid was immunogenic, C57Bl/6 mice were immunized with 25 μg of each plasmid 3 times at 2 week intervals. One week after final immunization, cellular and humoral responses were determined against consensus Glade A, B and C. All plasmids induced either a cellular or humoral responses; however there was variation between different plasmids (FIG. 14). For example, the highest cellular response as assessed by IFN-γ spot forming units (SFU) is plasmid A5 (Q23ENV17) (over 2000 SFU) and the lowest is plasmid C9 (Du156.12) (<100 SFU but above background) (FIG. 14A). Additionally, the regions of the antigen which stimulate T cell responses differ across plasmids. Cellular responses induced by Glade A Envs tend to be more reactive to the N-terminus peptides (pool 1) whereas responses to Glade B and C Env are spread across the protein (FIG. 14A). Humoral responses induced by these plasmids were also determined using consensus Glade A, B, and C gp120 and gp140 proteins as well as HXBC2 gp41 (FIG. 14B, FIG. 14C and FIG. 14D). Similar to the cellular responses, a wide range of binding reactivity across the plasmids was observed. Surprisingly, certain plasmids like B2 (REJO4541.67), B4 (TRJO4551.58), C1 (CAP45.2.00.G3), and C5 (ZM233M.PB6) which induces strong cellular responses, do not induce any humoral responses against consensus proteins. While not being limited to any particular theory, this could potentially be due to the lack of consensus proteins expressing the binding epitope; the binding epitope induced by each plasmid is conformational; or a lack of overall humoral responses. In contrast, there are plasmids which induce both strong humoral and cellular responses like A6 (Q259d2.17), B1 (WITO4160.33), B5 (CAAN5342.A2), C7 (ZM214M.PL15), and C11 (Du172.17).

### Formulation of Plasmids Affects the Strength of the Response

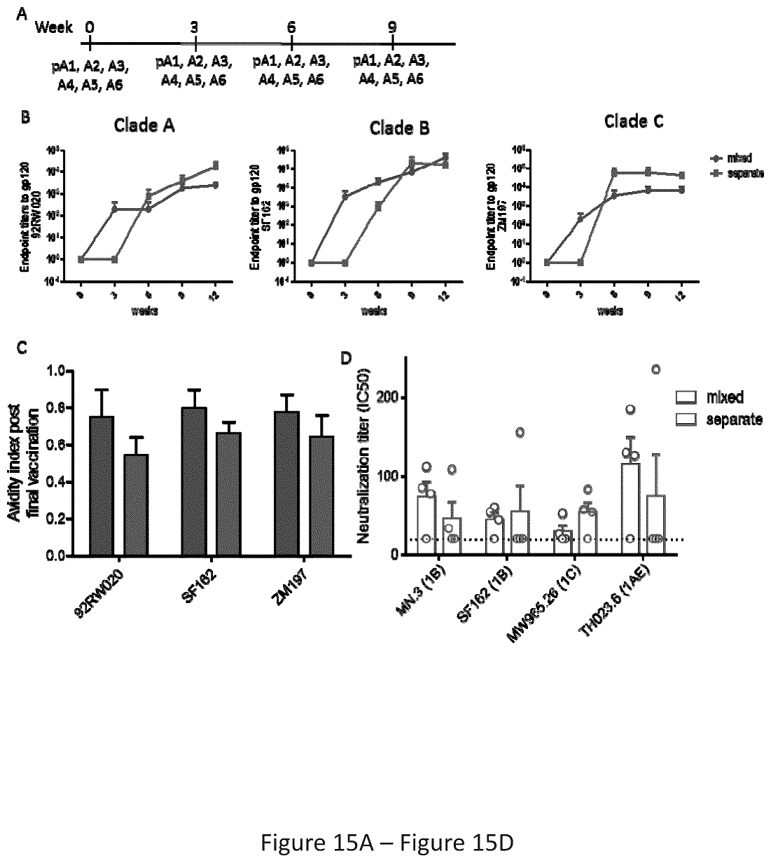

It was next sought to determine if multiple plasmids expressing the Glade A primary Envs could be formulated together and delivered to increase the breadth of antibody responses. However, questions arose as to if there would be antigen competition between the groups of Envelopes and thus, two vaccination regimens were performed: one where all of the plasmids were formulated together and another were each plasmid was given in a separate site. Guinea pigs were immunized four times with 100 μg of each plasmid ID followed by electroporation (FIG. 15A). The total amount of DNA for each immunization was the same across both groups (600 μg total−100 μg/plasmid) and the route and electroporation protocol were the same. The only difference was whether or not the plasmids were immunized separately or mixed together. Endpoint binding titers to the same primary gp120s were used to determine the induction of humoral responses. Though at the end of the vaccination (week 12) binding titers between the mixed vs separate are similar, the induction of humoral responses is quicker in the mixed group than in the separate group (FIG. 15B). Avidity of humoral responses was assessed at week 12 to determine if there was any difference between the two vaccination groups (FIG. 15C). The avidity index to 92RW020, SF162, and ZM197 were all slightly higher, though not significantly different, in the guinea pigs which received the mixed formulation. In addition, post final vaccination neutralization titers were slightly, though not significantly, higher in the mix vs separate group for three different tier 1 viruses (MN.3, SF162, and TH023.6) (FIG. 15D). This data suggest that mixing the Envelopes together does not dampen the humoral responses but instead, increases the initial seroconversion rate and could induce more superior functional antibody titers. Due to this and the ease mixed formulation provides for vaccine administration, all further studies were performed in this fashion.

Multiple Env Plasmids are Expressed in the Same Cells within the Skin

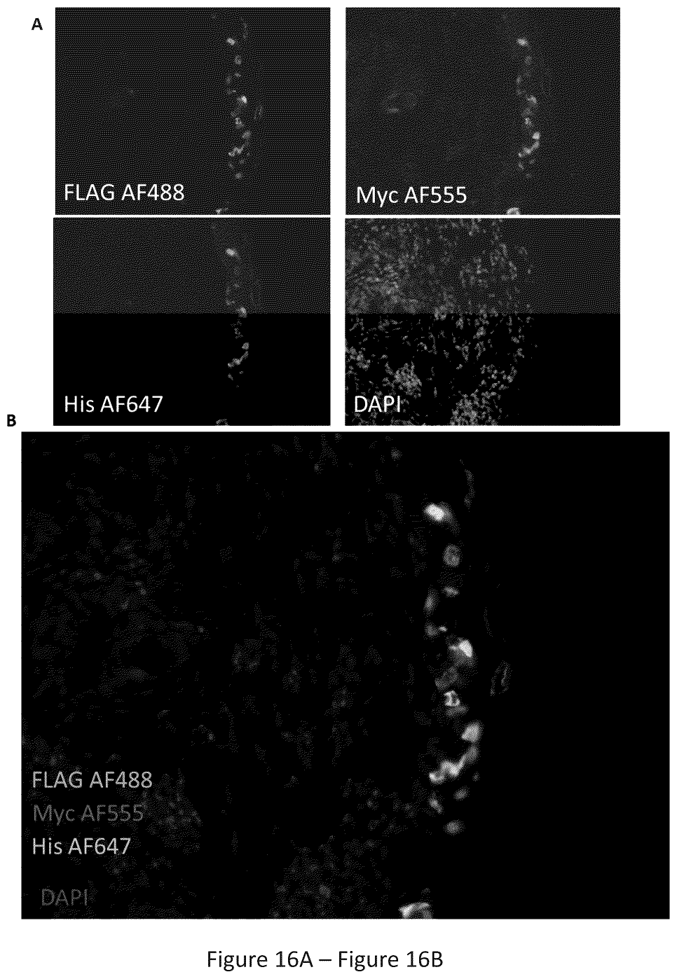

In order to determine if multiple Envelopes were being expressed in the same cell, tags were added to three different plasmids to efficiently detect each Envelope. Three tags were added to the C-terminus of three existing constructs using plasmid mutagenesis. The three constructs were pQ168ENVe2-HIS, pQ23ENV17-FLAG, pDu151.2-MYC and all expressed in vitro (data not shown). Two guinea pigs were injected with 16.5 μg of each plasmid formulated together and delivered to the dermis followed by electroporation. Expression of all constructs can be detected after 24 hours after injection (FIG. 16A). Importantly, there is overlap of fluorescent signal in multiple cells (FIG. 16B). This suggests that multiple constructs are being expressed in a single cell.

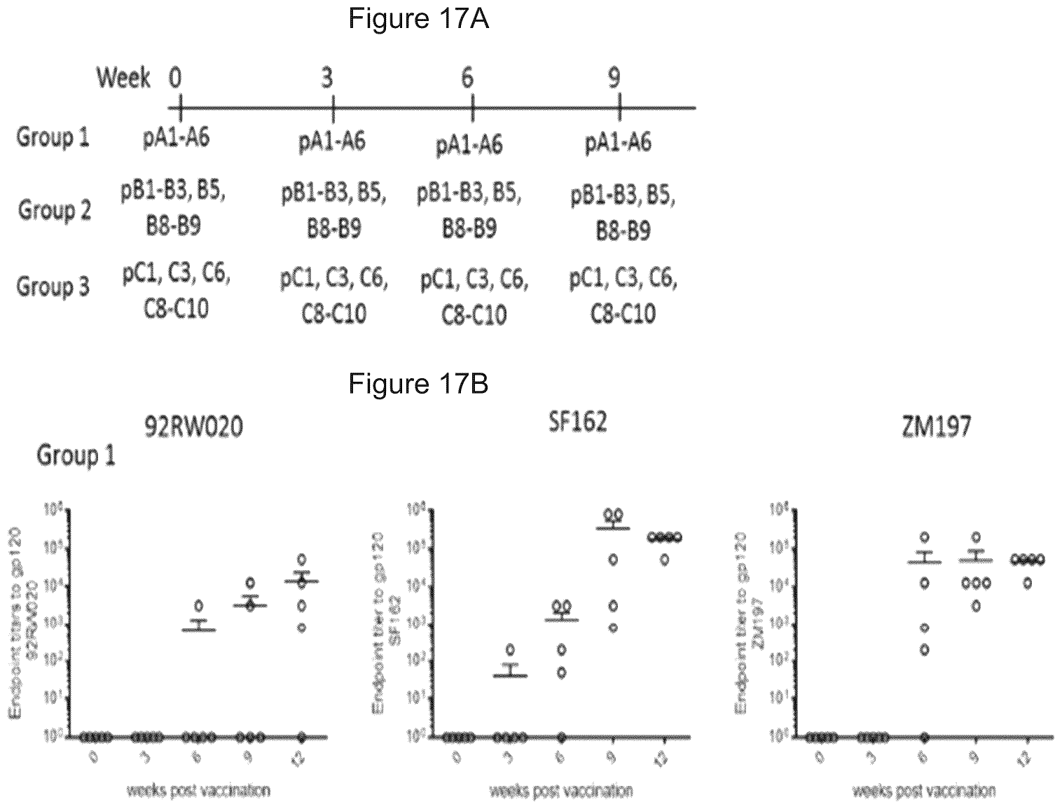

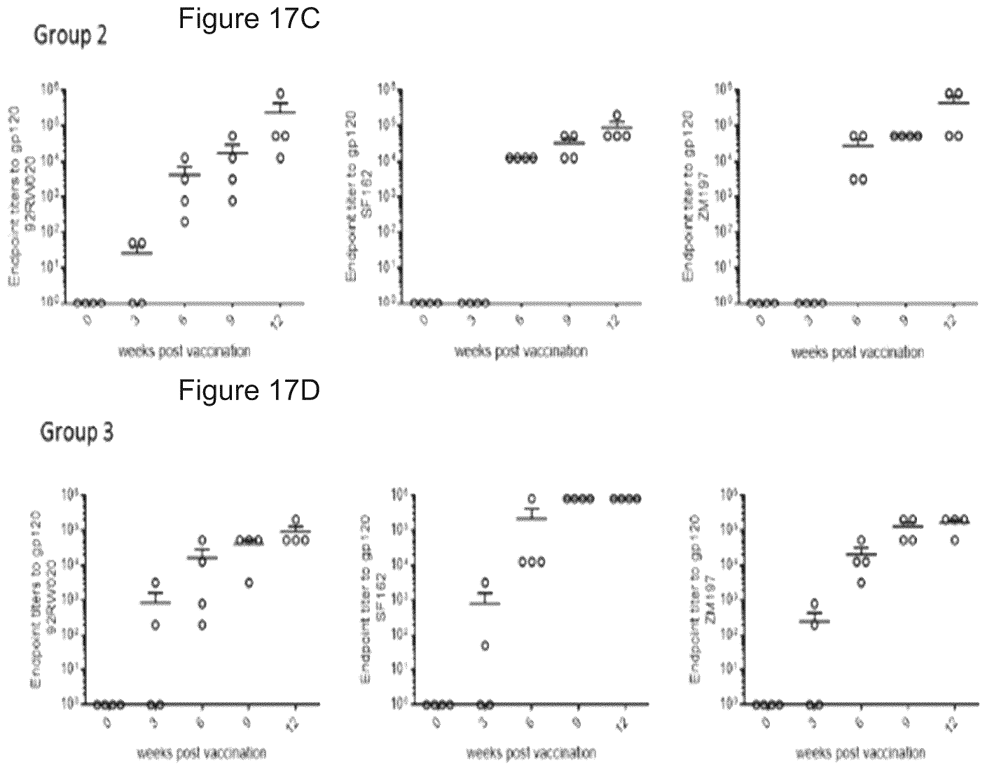

Groups of 6 Env Plasmids Induce Strong Humoral Responses in Rabbits To further investigate the use of small groups of primary Envelopes, groups of four rabbits were immunized with six plasmids expressing either Glade A, Glade B or Glade C Envs (FIG. 17A). All plasmids (100 μg/plasmid) were formulated together and delivered to six sites ID followed by electroporation. Binding titers against Glade A (92RW020), Glade B (SF162) and Glade C (ZM197) were assessed for each group of immunized rabbits over time (FIGS. 17B, 17C and 17D). After a single immunization, half of the animals immunized with Glade C Envs seroconvert to Glade A, B, and C gp120 proteins (FIG. 17D). By the second immunization, all animals immunized with Glade B and C Envs seroconverted to all gp120s (FIGS. 17C and 17D). Humoral responses in the rabbits immunized with Glade A Envs took slightly longer than with Glade B and C combinations but eventually did induce strong binding titers to all 3 gp120s (FIG. 17B) Humoral responses are boosted by each immunization reaching peak titers 3 weeks after final immunizations. Even though the animals are immunized with only a single Glade, all rabbits induce strong cross-Glade binding titers. In fact, the Glade C immunized rabbits had the highest binding titer responses to the Glade B (SF162) gp120 protein. Overall, formulating multiple primary transmitter founder or acute Envelopes together in a single formulation induces strong cross-Glade binding titers.
Increasing Diversity within Group Expands Antibody Responses

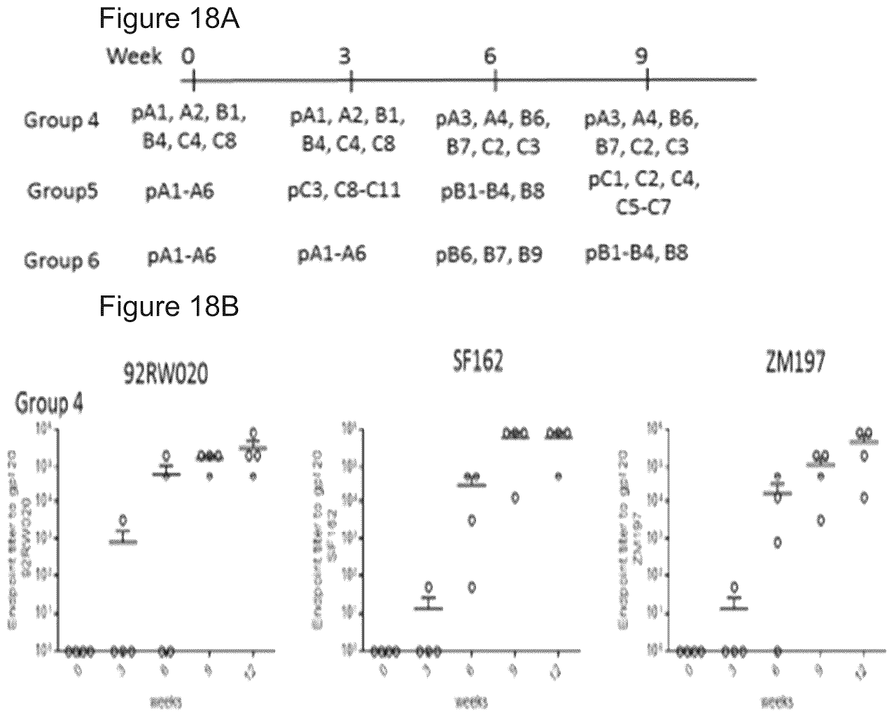

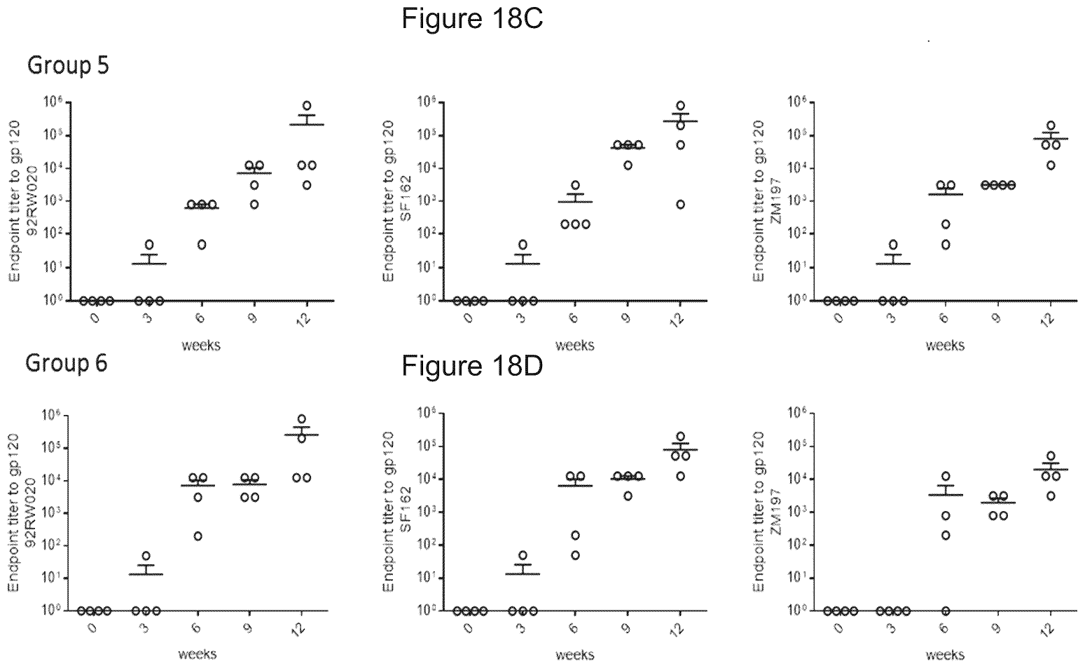

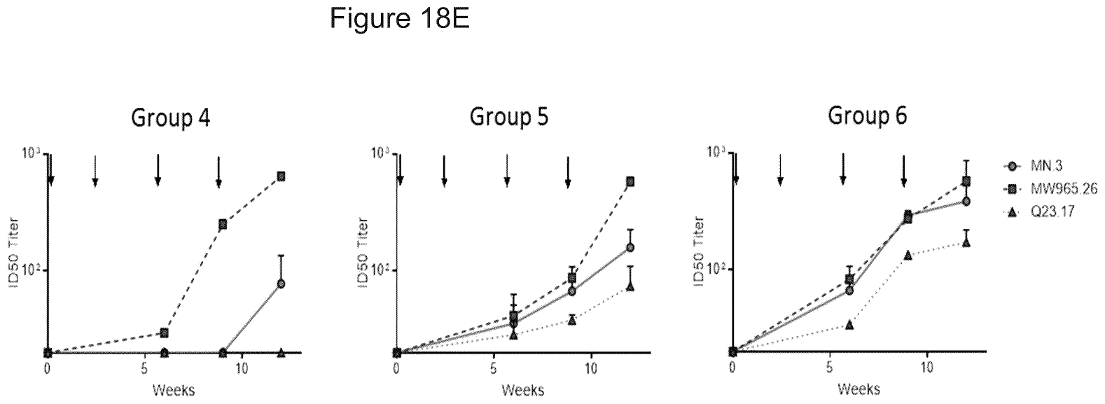

To investigate whether the results seen in the single Glade immunizations could be further expanded upon, two different groups of plasmids were used each containing two Glade A, B, and C primary gp160 Envelopes. Four rabbits were immunized with combination 1 (pA1, A2, B1, B4, C4, C8) twice followed by combination 2 (pA3, A4, B6, B7, C2, C3) (FIG. 18A). The plasmids were all formulated together per different combination with 100 μg (600 μg total) of DNA construct used per immunization, delivered ID followed by electroporation. The mean diversity within the groups was 22.0% and 21.0% respectively. The mean diversity between the groups was 20.6%. Once again after two immunizations, there is potent induction of binding titers against primary Glade A, B, and C gp120s (FIG. 18B). Neutralization titers were assessed over time against tier 1 viruses (MN.3, MW965.26 and Q23ENV17) (FIG. 18E). The highest neutralization titers were observed against MW965.26 on weeks 9 and 12. Limited responses were detected against MN.3 with no responses induced against Q23ENV17. The combination of plasmids expressing two Glade A, B, and C gp160s does appear to induce potent binding titers but limited neutralization breadth.

Creating “Clouds” with Limited Diversity Expands the Neutralization Breadth of Sera

It was next investigated if limiting the diversity within a “cloud” could enhance responses. Using the same six Glade A plasmids (pA1-A6) as a priming dose, four rabbits were immunized with additional “clouds” or groups of plasmid which were more limited in diversity and stayed within clades (FIG. 18A). The intra-cloud diversity ranged from 12.4-16.4% and inter-cloud was consistently around 20%. Each immunization was between 500 μg-600 μg of total DNA (100 μg of each plasmid) mixed together and administered ID to five or six separate sites followed by electroporation. Using this limited intra-cloud diversity regimen did not disrupt the ability to induce potent cross-Glade binding tiers against the three primary isolate gp120 (FIG. 18C). There is a consistent boosting of titers after every immunization with the highest binding titers obtained after the final immunization at week 12. Neutralization titers demonstrated stronger kinetics of induction and higher titers compared to group 4 (A, B, C mixed) (FIG. 18E). In comparison to group 4 (A, B, C mixed together), group 5 induced responses to MW965, MN.3 and Q23ENV17 after the second immunization and continued to increase after final immunization. The ability to induce this robust of a response by DNA alone has yet to be seen and could lend itself well to further expansion by boosting with a different platform.

Highest Induction of Robust Antibody Responses in Rabbits Primed Twice with the Same “Cloud”

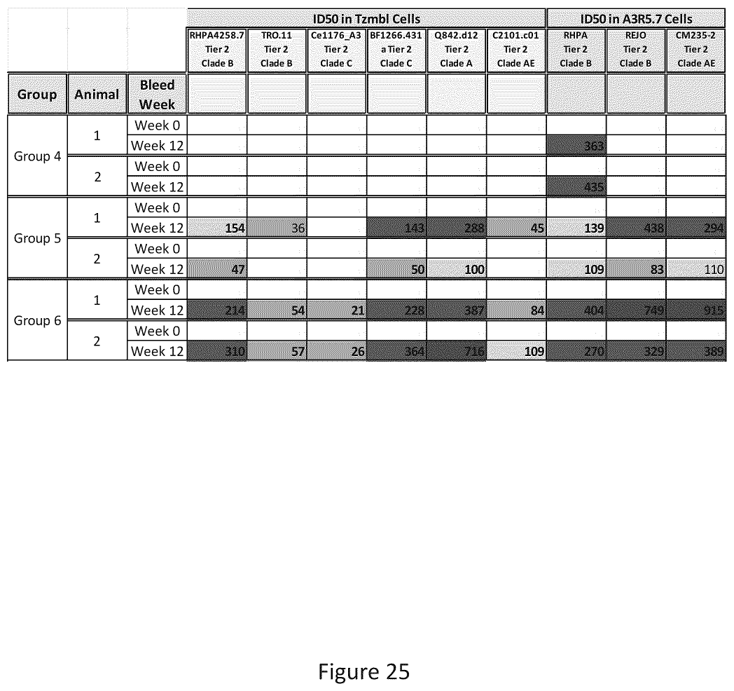

The final group of rabbits looked to determine if these responses would increase by priming with the same group twice. This would allow for the immune system to potentially honing in on specific epitopes which would later be expanded by boosting with additional clouds. Rabbits were immunized twice with the Glade A plasmids (pA1-A6) and boosted with two different groups of primarily Glade B immunogens (FIG. 18A). The intra-cloud diversity ranged from 13.3-14.3% and the inter-cloud diversity between 14-17.6%. Thus this regimen has the lowest diversity between the clouds compared to the other two combinations. This low intra-cloud diversity did not limit the responses, as potent binding titers are induced in all animals after two immunizations (FIG. 18D). The highest and quickest induction of neutralization is seen for this group, with the most powerful response happening after the final immunization (FIG. 18E). In addition, sera from two rabbits were able to neutralize more isolates at higher IC50 concentrations than groups 4 and 5 (FIG. 25). This includes hard to neutralize tier 2 viruses where only one virus (Ce1176_A3) is not able to be neutralized. Thus, priming rabbits with two immunizations of same group of plasmids seems to focus the immune system in a way that allows for effective induction of broadly binding and neutralizing antibodies.

Non-Human Primates Immunized with “Clouds” of Primary Envelopes Induce Potent Cellular Responses

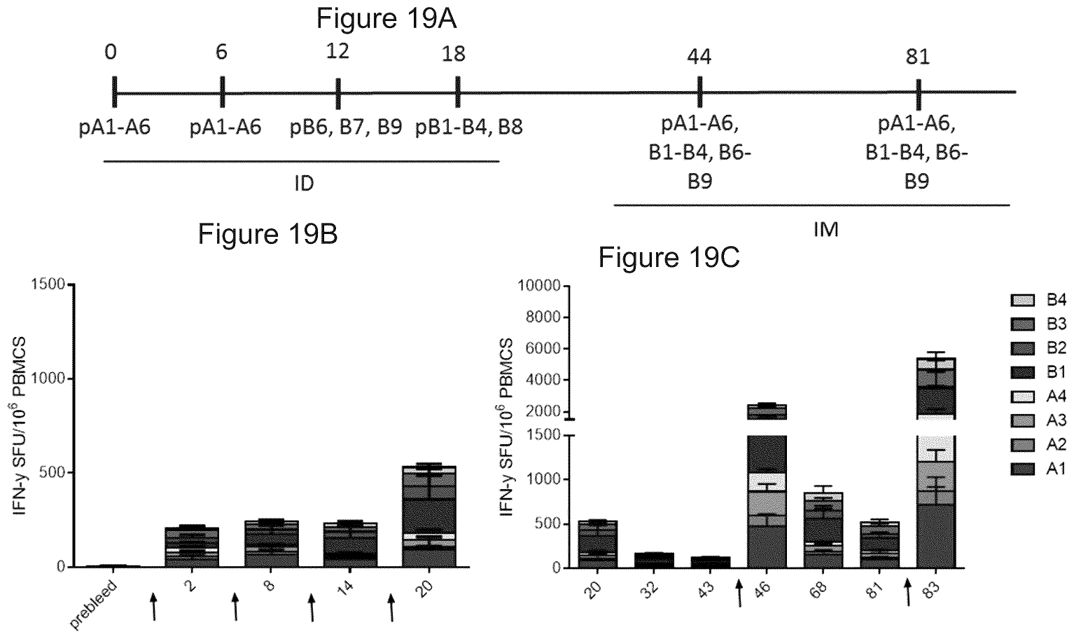

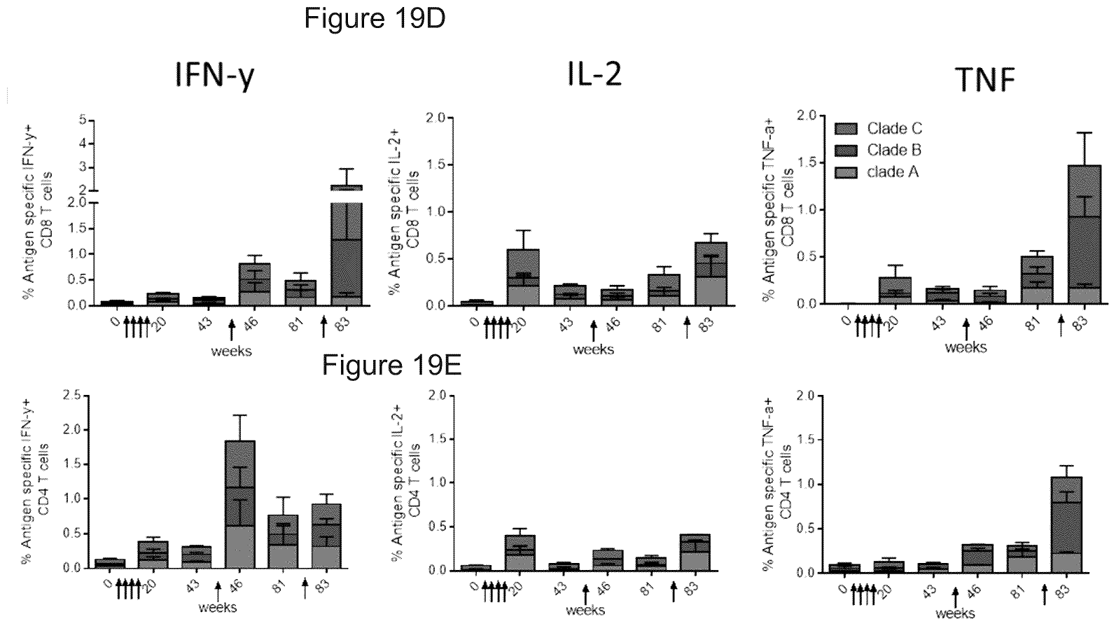

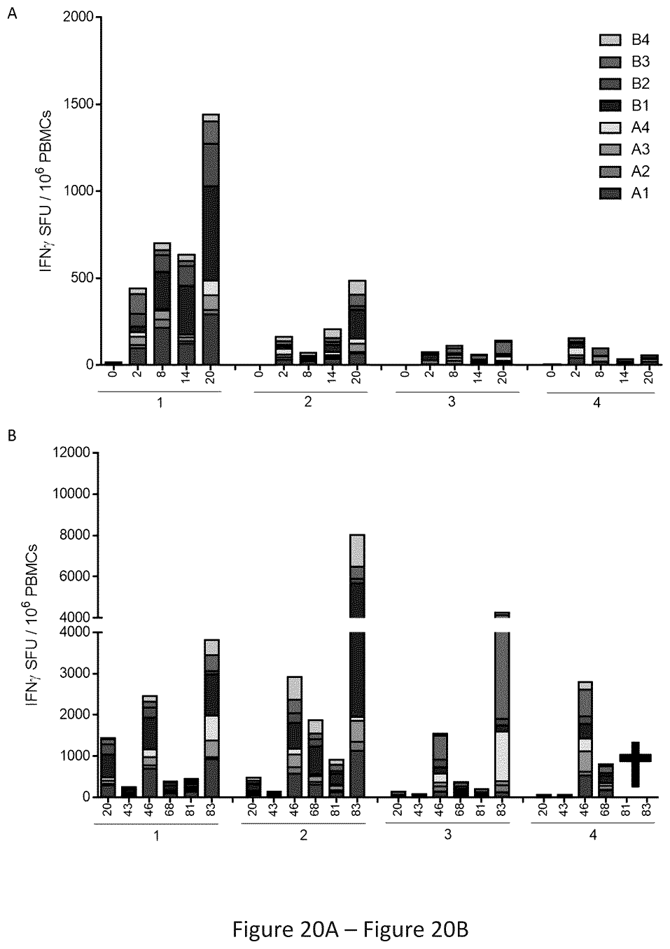

To further characterize the vaccine induced responses produced by the most potent regimen, four rhesus macaques (RhMs) were immunized with a similar vaccine regimen (FIG. 19A). On weeks 0, 6, 12 and 18, the NHP received a mixture of different Envelopes (1 mg/plasmid) formulated together and delivered ID followed by electroporation. To further expand the vaccine induced responses, at weeks 44 and 81 post first vaccination, all animals received all of the Envelopes from vaccination 1-4 (1 mg/plasmid) delivered IM at a single site followed by electroporation. Cellular and humoral responses were followed two weeks after each vaccination. After only a single immunization, IFN-γ spot forming units (SFU) are detected against consensus clades A and B peptides (FIG. 19B). These responses are not boosted with the second or third immunization of the priming cloud but are expanded upon after the fourth immunization. After the final ID immunization, the average total IFN-γ SFU is around 500 SFU with even distribution of reactivity between Glade A and B (range 100-1,500 SFU) (FIG. 20A). Though there is contraction into the memory phase (weeks 32 and 43), cellular responses can still be detected against consensus Glade A and B almost 6 months (week 43) after final ID immunization (FIG. 19C). After the first IM boosting immunization at week 44, cellular responses expand greatly to levels over quadruple the amount seen after final ID immunization. Over eight months after IM immunization (week 81), cellular responses have contracted but remain around the levels seen after final ID immunization. Upon second IM boost, cellular responses again expand above those seen after the previous IM immunization with IFN-γ SFU averaging around 7000 (responses varying from 4000-10,000 SFU) (FIG. 20B). These responses are extremely high, especially since they are against unmatched peptides. In addition, since consensus peptides are used, this suggests that these small “clouds” of immunogens are able to induce potent cellular responses against conserved regions within the Envelope. This could be important for the induction of cytotoxic T cells as well as providing broad CD4 T cell help.

To further explore the cellular responses induced by the primary Envelope cloud immunization, intracellular cytokine staining was performed using consensus Glade A, B and C peptides. CD8 T cell responses after ID immunization (week 20) primarily express IL-2 and TNF-α with limited IFN-γ production (FIG. 19D). Each IM immunization increased the percent of CD8 T cells expressing IFN-γ. An additional increase in TNF-α production is also seen after the final IM immunization (week 83). In contrast, the IL-2 production observed after final ID immunization is not boosted by either IM immunization and levels after final IM immunization are the same as after final ID immunization. CD4 T cell responses were also assessed against Glade A, B and C peptides (FIG. 19E). The percent of CD4 T cells expressing IFN-γ and IL-2 is relatively the same after the ID immunization (week 20) with a lower percentage of CD4 T cells expressing TNF-α. Similar to CD8 T cells, the proportion of CD4 T cells secreting IL-2 remains relatively consistent across time with slight waning at each memory time point. However, after the first IM immunization, there is a sharp increase in CD4 T cells secreting IFN-γ. Similar boost is not observed after the second immunization. Expression of TNF-α remains consistent into memory after ID immunization, is boosted by the first and second IM immunization. Importantly, similar to ELISpots, potent cytokine secretion was observed after stimulation with cross-Glade consensus peptides. Though these NHPs were only immunized with Glade A and B primary Envs, cellular responses against consensus Glade C peptides are detected at similar levels to Glade B responses.

### Binding and Functional Antibodies Induced Using Primary Env DNA Vaccination

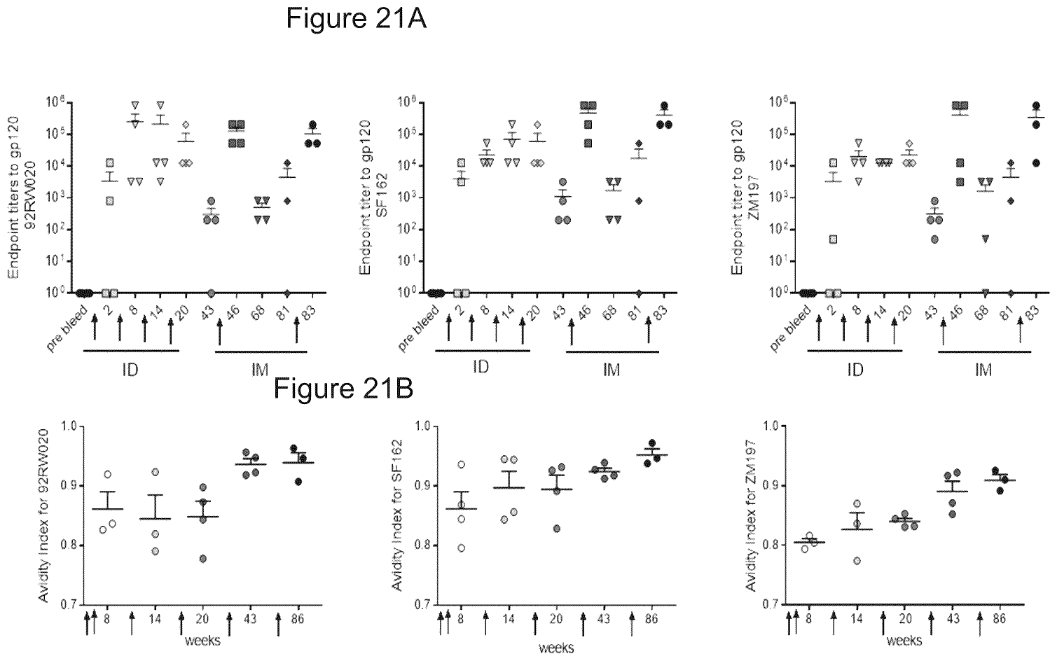

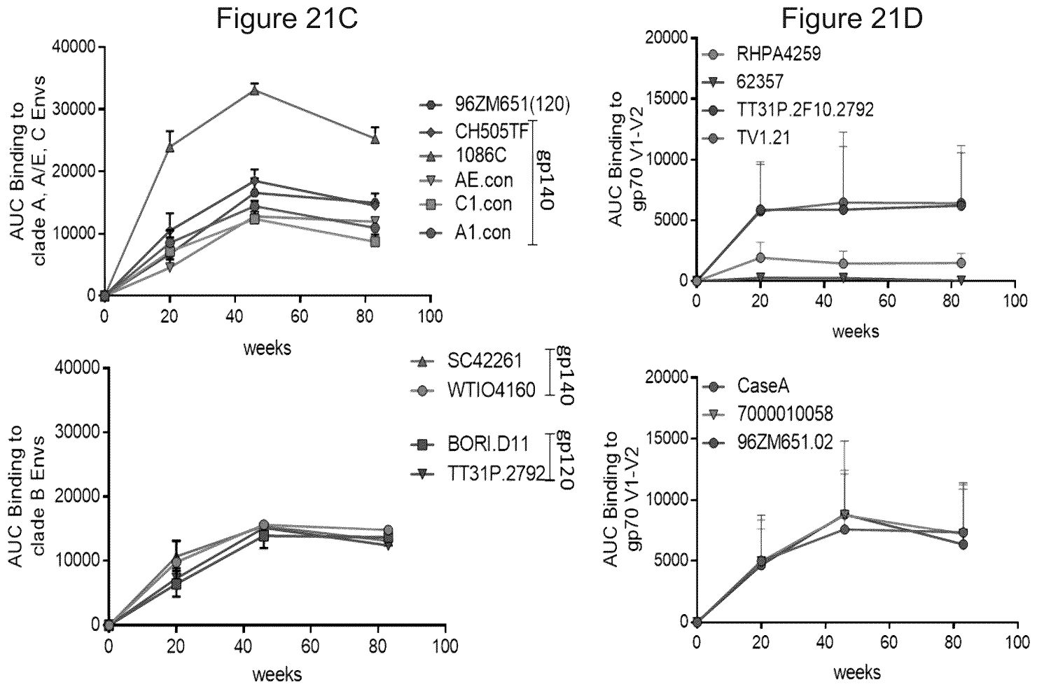

The primary Envelope cloud immunization also induces potent humoral responses. After a single immunization, two out of eight RhMs seroconvert to Glade A, B and C gp120 proteins (FIG. 21A). After the final ID immunization, all animals have strong endpoint binding titers against the primary Envelopes averaging above 10⁴. Similar to cellular responses, binding titers also contract down in the memory phase but remain high (average above 10³) six month post last ID immunization (week 43). Also similar to cellular responses, after the IM boost, binding titers reach levels higher than after ID immunization with the average binding titer above 10⁵. These responses are also slightly boosted after a second IM immunization to levels reaching 10⁶. Strong avidity indexes of around 0.8 are induced after the second ID immunization (FIG. 21B). However, subsequent ID immunization did not improve the avidity index. The first IM boost increased the avidity index across all three gp120 proteins with minimal to no increase in avidity after the second IM immunization. To further explore the binding capacity of the humoral responses induced, binding to consensus and primary gp120 and gp140s was determined using binding antibody multiplex assay (BAMA) (FIG. 21C). Strong binding titers against Glade A, B, C and AE Envs were detected with the highest responses obtained after the first IM immunization. The strongest binding response was detected against the primary isolate gp140 Env 1086c, with almost 3 fold higher area under the curve (AUC) binding compared to other Envs. V1/V2 binding against multiple different gp70 scaffold was also assessed (FIG. 21D). Interestingly there were three binding patterns to V1/V2 scaffolds which emerged. The first is binding kinetics similar to that which was observed in the binding to the whole protein with induction by the final ID immunization, peak after second IM immunization and similar levels after the second IM immunization (FIG. 21D, bottom graph). The second pattern is induction of binding after ID immunization but no boosting after each IM immunization (FIG. 21D, top graph—TT31P and TV1.21). The final pattern is limited to no induction of binding (FIG. 21D, top graph—RHPA4259 and 62357). These differences in binding patters could help suggest a potential target epitope.

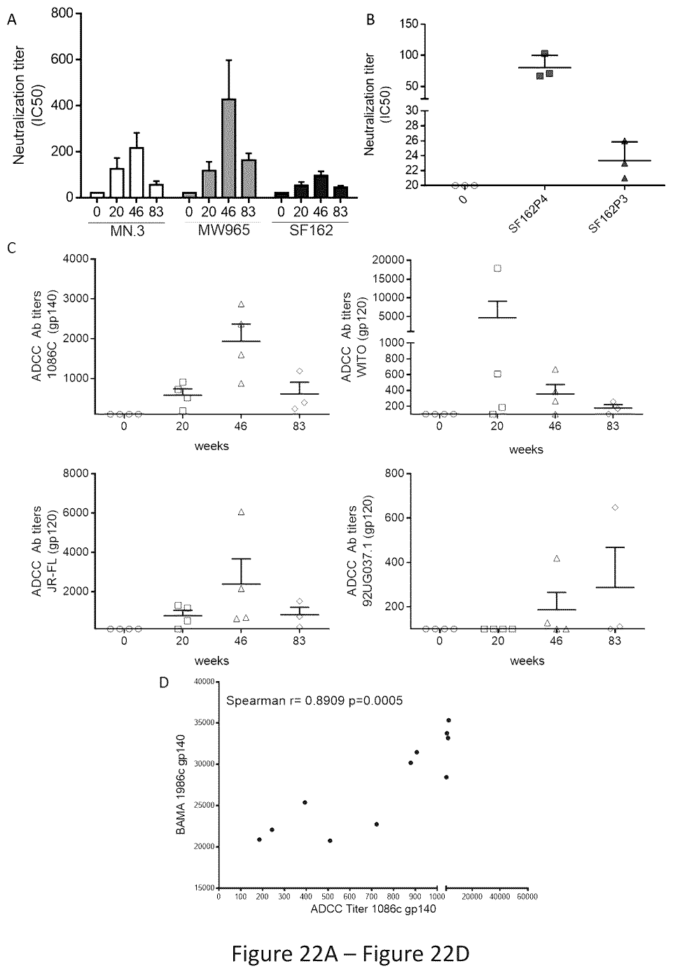

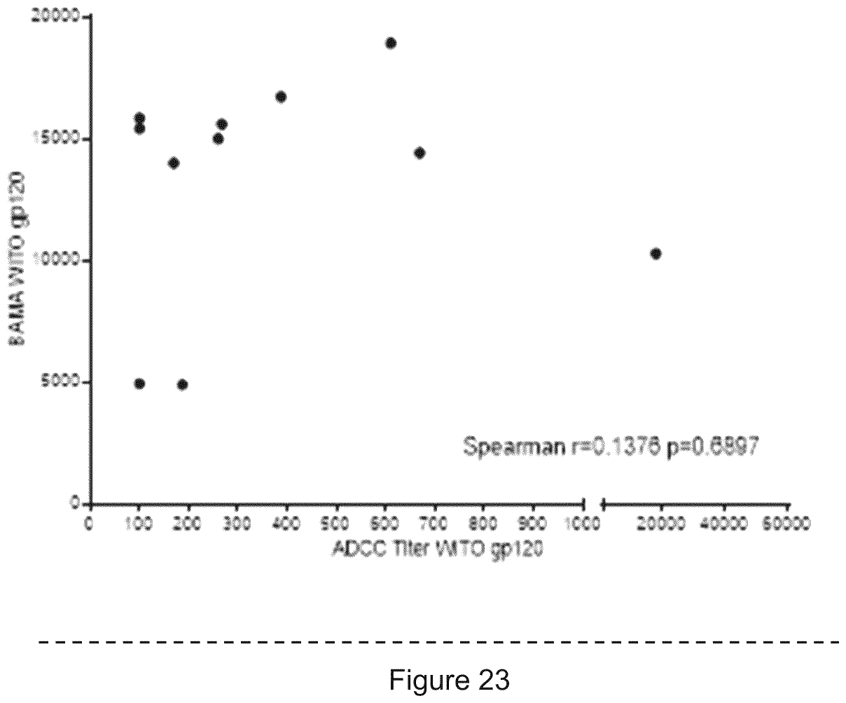

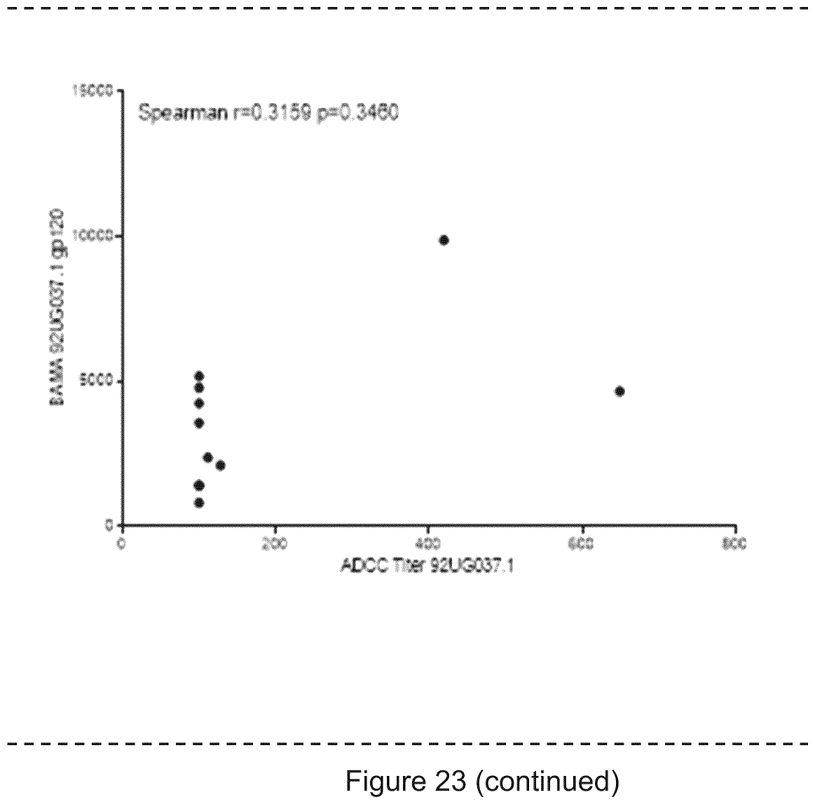

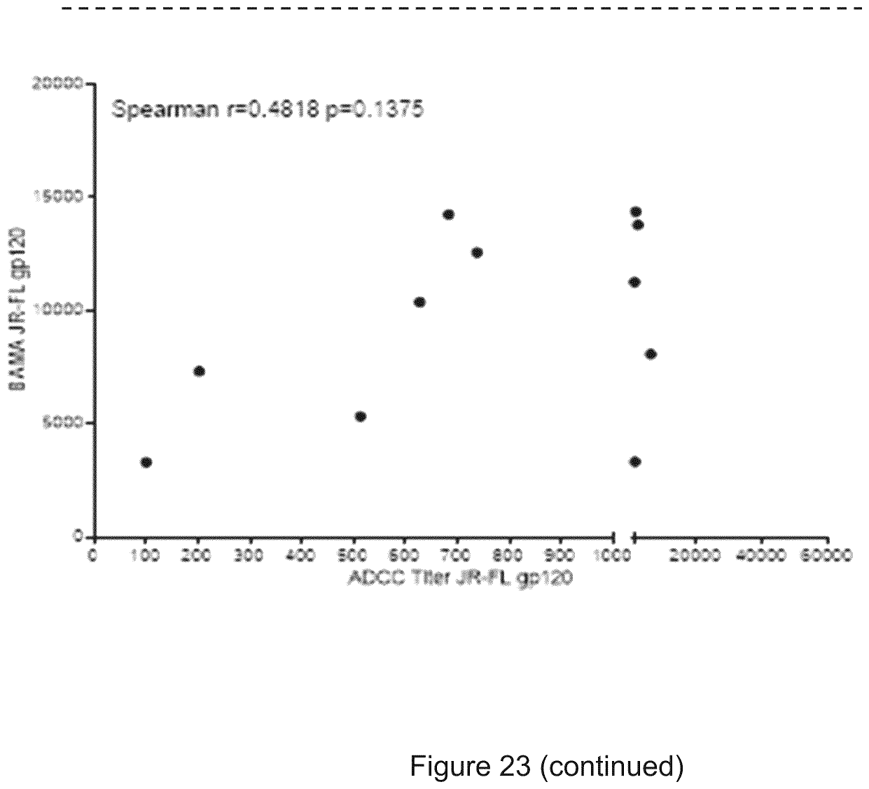

In addition to binding titers, the vaccination regimen also induces functional antibodies. Using only DNA vaccination cross Glade neutralization titers against a diversity of tier 1 viruses is achieved (FIG. 22A). After ID immunization, neutralization titers for MN.3, MW965 and SF162 average above or around 10². After the first IM boost, levels are increased to above 10³ for MN.3 and MW965 and just below 10³ for SF162. After the second IM boost levels increase are not seen above those observed after the initial IM boost. In fact, for MN.3, MW965 and SF162, the levels were lower and usually averaged around the same titers as those seen after the ID immunizations. However, levels against SF162P4 IMC were detected and importantly, there were limited but low neutralization titers induced against the tier 2 virus SF163P3 after final IM immunization (FIG. 22B). Since the role of antibodies with ADCC capabilities has been sμggestive in protection against HIV infection (RV144 correlates analysis), ADCC activity was tested against targets coated with 1086c (gp140), WITO (gp120), JR-FL (gp120) and 92MG037.1 (gp120) (FIG. 22C). Similar to V1/V2 binding, three different patterns of ADCC induction emerge. The first displays similar kinetics to BAMA, V1/V2 binding pattern 1 and neutralization titers with peak titers induced post 1^{st} IM immunization which were not further boosted after the 2^{nd} IM (1086c and JR-FL). The second pattern is observed with WITO coated targets where the strongest response was observed after the ID immunizations. If the one outlier is removed from the analysis, these responses are maintained with the first IM immunization but slightly decline with the second. The third pattern is seen with 92MG037.1 where only 1 or 2 NHPs are able to induce low ADCC activity against the target cells. Differences between these three Env could again suggest differences in binding epitopes and induction of certain humoral responses after each immunization. Interestingly, the AUC determined by the binding antibody multiplex assay and ADCC titers against 1086c correlated (spearman r=0.8909 p=0.0005) (FIG. 22E). However, similar correlations were not found for WITO, JR-FL and 92MG037.1 (FIG. 23). These data supports the use of primary transmitter founder Envelopes deliver in small “cloud” immunizations for the induction of potent cellular and humoral responses.

### Discussion

An effective HIV vaccine will likely need to induce both cellular and humoral responses. Previously, DNA vaccines have been able to induce potent cellular responses but lacked humoral responses. Advances in plasmid optimizations, formulation and delivery have significantly increased DNA vaccines ability to induce humoral responses. Here, it is explored the ability to use combinations of full length gp160 Envs which were isolated during the early/acute phase of infection (Li et al., 2006, J Virol 89:11776-90; Li et al., 2006, J Virol 79:10108-25; Wilen et al., 2011, J Virol 85:8514-27). All inserts were immunogenic in mice, displaying a range of cellular and humoral responses. Interestingly, there was not a consistent pool of peptides which was dominated across all antigens. Instead for clades B and C inserts, cellular immune responses were detected across the entire antigen. Similar breadth of responses is observed using our consensus antigens (Yan et al., 2011, Vaccine 29:7173-81). In contrast, the majority of Glade A Env inserts induced very strong responses against the N terminus (pool 1) and fewer responses across the rest of the protein. This could be due to the heterogous nature of the peptide used or a dominant epitope at the N terminus of the protein. Additionally, this dominance could be mouse specific as when a combination of these plasmids is administered into NHPs, responses to all 4 peptide pools is evident; however, pool 1 still dominates (FIG. 19 and FIG. 20).

The ability to induce protective responses against multiple serotypes is evident in the Influenza, Human Papillomavirus, and Pneumococcus vaccines (Ortqvist et al., 1998, Lancet 351:399-403; Harper et al., 2006, Lancet 367:1247-55; Paavoen et al., 2009, Lancet 374:301-14; Joura et al., 2015, NEJM 372:711-23; Osterholm et al., 2012, Lancet Infect Dis 12:36-44). Due to the breadth of HIV diversity is it likely that multiple antigens will need to be formulated into a single injection for ease of delivery. In terms of humoral responses, within this study, it is demonstrated that up to six plasmids can be combined together and lead to strong humoral responses than when delivered to individual sites. Using immunofluorescence and tagged constructs it is also shown that up to three constructs can be detected in the same cell. This leads to the possibility of heterotrimers, which have been previously show to induce stronger neutralization titers compared to homotrimers of Env proteins (Bowles et al., 2014, PLoS One 9:e114709; Sellhorn et al., 2012, J Virol 86:128-42). In theory, the diversity within these heterotrimers could focus the immune response on conserved epitopes. Importantly, it is within these conserved regions of HIV that broadly neutralizing antibodies targets (Kwong and Mascola, 2012, Immunity 37:412-5). However, these regions tend to be much less immunogenicity than the variable loops and thus are more difficult to target. It is important to follow up on whether or not DNA encoded gp160s can form in vivo heterotrimers and if so, how does the percent diversity between the Envs affect this ability.

In addition to exploring multiple different combinations of HIV Envs, different sites of delivery were also used. Advances in electroporation technology have allowed for multiple different tissues to be targeted included the traditional, intradermal and intramuscular (Sardesai and Weiner, 2011, Curr Opin Immunol 23:421-9). Within this study, NHP were vaccinated with four ID immunizations followed by two IM boost. These two sites have different cellular composition and thus could produce unique vaccine induced responses. A single ID immunization is able to induce strong cellular responses and seroconversion in 50% of the animals (FIG. 19A and FIG. 21A). Interestingly, even though cellular responses do not boost with the 2^{nd} and 3^{rd} ID immunization, continual improvement in humoral responses were observed. However, though binding titers improve over the ID immunizations, the avidity of these antibodies remains fairly consistent only increasing upon IM immunization (FIG. 21A and FIG. 21B). A sharp increase in cellular responses were observed after the first IM immunization, with both CD4 and CD8 T cells expressing IFN-γ. This boost in responses was also observed in both binding and functional antibody titers (FIG. 21 and FIG. 22). Unexpectedly, these functional antibody titers did not further increase after the second IM immunization but instead were at levels similar to after the ID immunizations. Following the second IM immunization, CD8 T cell dominate IFN-γ production with a decrease in CD4 T cell production compared to after the 1^{st} IM immunization (FIGS. 19D and 19E). Here it is demonstrated for the first time that ID DNA immunization can be further expanded by IM immunization. The ability to understand how the different sites of immunization skews the immune response and how boosting can affect memory cell activation is imperative for DNA vaccine development.

Numerous different plasmids expressing consensus, chronic and acute/early Envs have been developed. It is demonstrated herein that guinea pigs and rabbits exposed to groups of immunogens are able to induce strong binding titers to heterologous Envs and different clouds of plasmids can influence the kinetics of tier 1 neutralization induction. Additionally, combinations of 14 different Env plasmids were able to induce strong cellular and humoral responses. Importantly, these humoral responses were functional after only DNA vaccination. Determining what combination of Envs produces the strongest and broadest responses is imperative for the HIV vaccine development field.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising two or more nucleic acid molecules encoding an HIV immunogen, wherein each nucleic acid molecule comprises a sequence independently selected from the group consisting of: a nucleic acid sequence encoding a sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 52, 54, 56, 58, 60, and 62; a nucleic acid sequence encoding a fragment of one selected from the group consisting of SEQ ID NOs: 2, 4, 6, 8, 10, 12, 14, 16, 18, 20, 22, 24, 26, 28, 30, 32, 34, 36, 38, 40, 42, 44, 46, 52, 54, 56, 58, 60, and 62; a nucleic acid sequence encoding a sequence that is at least 90% homologous to a sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 52, 54, 56, 58, 60, and 62; and a nucleic acid sequence encoding a fragment of a sequence that is at least 90% homologous to a sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 52, 54, 56, 58, 60, and 62; wherein the nucleic acid sequence is optionally liked to a to a nucleic acid sequence encoding an IgE signal peptide.

2. The composition of claim 1, wherein each nucleic acid molecule comprises a sequence encoding a sequence independently selected from the group consisting of SEQ ID NOs: 2, 4, 6, 8, 10, 12, 14, 16, 18, 20, 22, 24, 26, 28, 30, 32, 34, 36, 38, 40, 42, 44, 46, 52, 54, 56, 58, 60, and 62.

3. The composition of claim 1, wherein each nucleic acid molecules comprises a sequence independently selected from the group consisting of: a nucleic acid comprising a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, and 61; a nucleic acid comprising a fragment of a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, and 61; a nucleic acid comprising a sequence at least 90% homologous to a sequence selected from SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, and 61; and a nucleic acid comprising a fragment sequence at least 90% homologous to a sequence selected from SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, and 61.

4. The composition of claim 3, wherein each nucleic acid molecule comprises a sequence independently selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, and 61.

5. The composition of claim 1, wherein the composition comprises 3 or more nucleic acid molecules.

6. The composition of claim 1, wherein the composition comprises 6 or more nucleic acid molecules.

7. The composition of claim 1, wherein the composition comprises 10 or more nucleic acid molecules.

8. The composition of claim 1, wherein the composition comprises 14 or more nucleic acid molecules.

9. The composition of claim 1, wherein the composition comprises two or more plasmids, wherein each plasmid comprises only one of SEQ ID NOs: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, and 61.

10. The composition of claim 1 formulated for delivery to a subject using electroporation.

11. A method of immunizing a subject in need thereof against HIV, the method comprising administering a first vaccine comprising one or more nucleic acid molecules comprising a sequence independently selected from SEQ ID NO: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, and 61, a variant thereof or a fragment thereof.

12. The method of claim 11, the method further comprising administering a second vaccine comprising one or more nucleic acid molecules comprising a sequence independently selected from SEQ ID NO: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, and 61, a variant thereof or a fragment thereof.

13. The method of claim 12, the method further comprising administering a third vaccine comprising one or more nucleic acid molecules comprising a sequence independently selected from SEQ ID NO: 1, 3, 5, 7, 9, 11, 13, 15, 17, 19, 21, 23, 25, 27, 29, 31, 33, 35, 37, 39, 41, 43, 45, 47, 49 51, 53, 55, 57, 59, and 61, a variant thereof or a fragment thereof.

14. The method of claim 11, wherein the first vaccine is administered intradermally.

15. The method of claim 12, wherein the second vaccine is administered intradermally.

16. The method of claim 13, wherein the third vaccine is administered intramuscularly.

17. The method of claim 11, wherein the first vaccine is administered twice.

18. The method of claim 12, wherein the second vaccine is administered twice.

19. The method of claim 13, wherein the third vaccine is administered twice.

20. A method of preventing HIV infection in an individual comprising administering a prophylactically effective amount of the composition of claim 1 to an individual.


SEQUENCE LISTING (62 sequences)

SEQ ID NO: 1 - DNA, 2583 bp, synthetic construct
  misc_feature 1..2583: note = pGX1025 - Env Clade A tier 2 Q769ENVd22 DNA Sequence
  source 1..2583: mol_type = other DNA
atgagggcaatgggcattcagagaaactggcagaacctgtggagatggggcactatgatcctgggcatgatcctgatttgttgtagcgccgctgggaacctgtgggtgaccgtctactatggagtgcccgtctggcgggacgctgaaaccacactgttctgcgcaagcgacgcaaaggcctacgatagagaggcccacaacgtgtgggctactcatgcatgcgtgccaaccgatccaagcccacaggaggtgcctctgggcaacgtcaccgaggagttcaacatgtggaagaacaatatggtggaacagatgcacacagacatcatttctctgtgggatcagagtctgcagccttgcgtgaaactgacaccactgtgcgtcactctgaactgttcaaatagcaacaatattccatccgtgtctaacatcaccgacgatatgaaggaggaaatcaaaaactgttccttcaatatgactaccgagctgaaggacaagaaacagaacgtgtactctctgttttatcggctggatgtggtccccctggagaccaaaacaaaccagaatagctcccactcacgataccggctgattaactgcaatacaagcgccatcactcaggcatgtcctaaggtgtccttcgagcctattccaatccattattgcgctccagcaggcttcgccattctgaagtgtaacgacaaagggtttaatggaacaggcctgtgcaagaacgtgagcaccgtccagtgtacacatggcatcaaacctgtggtcagcactcagctgctgctgaatgggtccctggccgaaggcaaagtgatggtgcggagcgagaacatcacaaacaacgctaagaacatcatcatccagttcaacaattcagtgcagattaactgcacacggccaggaaacaatactagaaagagcatccacctggggcccggaaaagtgttttacgccaccgacattatcggcgatatcagaaaggctcattgtaacgtgaataggcagcagtggaacaaaactctgcaggacgtggccactcagctgagaacccacttcagaaacaggaccatcatctttaacaactctctgggcggggatctggaaattacaactcatagtttcaactgcaggggcgagttcttttactgtaatacatctgggctgtttaacggaatctggaatggcacccaggaacctaaccgcacagagagtaatgacactattaccctgcagtgccgcatcaagcagattatcaacatgtggcagcgagtgggacaggccatctatgctccccctattcagggcgaaatcaggtgtgagagtaacattaccgggctgatcctgacacgcgatggaggcattatcaattcaactgaggaaaccttcaggccaggaggaggcgacatgcgagataactggcgatctgaactgtacaagtataaagtggtcaagatcgagccactgggagtggcaccaaccaaggctaaacggagagtggtcgaacgagagaaacgggccgtgggcttcggggctttctttctgggatttctgggcgcagccgggagtacaatgggagctgcatcaatcacactgactgtgcaggccaggcagctgctgagcggcattgtccagcagcagaacaatctgctgcgcgcaatcgaggcccagcagcacctgctgaagctgaccgtgtggggcatcaaacagctgcaggcaagggtgctggcagtcgagcggtacctgaaggaccagcagctgctgggaatttggggctgcagcggcaagttcatctgtaccacaactgtgccctggaactctagttggtccaataagagtcagtcagaaatctgggacaacatgacatggatgcagtgggataaggagattaacaactacactcagatcatctatgacctgatcgaggaatcccagcggcagcaggaaaagaacgagcaggacctgctggcactggataaatgggccaacctgtggaattggttcgatatctctaattggctgtggtacattaagatctttattatgatcgtggggggactgattgggctgcggatcgccttcgctgtgctgagcgtcatcaaccgcgtgcgacagggatatagccccctgtcctttcagacccacacacccaatcctagagacctggatagacctggcaggattgaggaagagggcggggagcaggaccgggatagatcaatccgactggtgagcgggttcctggcactggcctgggacgatctgagatccctgtgcctgttctcttatcacaggctgcgcgacttcatcctggtggccgctaggaccgtcgaactgctgggccatatcagcctgaagggactgaggcgaggatgggagggcctgaaatacctgggaaacctgctgtcttattggggccgcgaactgaagattagtgccatcaatctgctggacactattgctatcgtggtcgcagaatggaccgatcgaattatcgagatcggccagcggctgtgtagagccattattaacattccaagacggattcgccagggatttgaaagagcactgctgtgataa

SEQ ID NO: 2 - protein, 859 aa, synthetic construct
  REGION 1..859: note = pGX1025 - Env Clade A tier 2 Q769ENVd22 Amino Acid Sequence
MRAMGIQRNWQNLWRWGTMILGMILICCSAAGNLWVTVYYGVPVWRDAETTLFCASDAKAYDREAHNVWATHACVPTDPSPQEVPLGNVTEEFNMWKNNMVEQMHTDIISLWDQSLQPCVKLTPLCVTLNCSNSNNIPSVSNITDDMKEEIKNCSFNMTTELKDKKQNVYSLFYRLDVVPLETKTNQNSSHSRYRLINCNTSAITQACPKVSFEPIPIHYCAPAGFAILKCNDKGFNGTGLCKNVSTVQCTHGIKPVVSTQLLLNGSLAEGKVMVRSENITNNAKNIIIQFNNSVQINCTRPGNNTRKSIHLGPGKVFYATDIIGDIRKAHCNVNRQQWNKTLQDVATQLRTHFRNRTIIFNNSLGGDLEITTHSFNCRGEFFYCNTSGLFNGIWNGTQEPNRTESNDTITLQCRIKQIINMWQRVGQAIYAPPIQGEIRCESNITGLILTRDGGIINSTEETFRPGGGDMRDNWRSELYKYKVVKIEPLGVAPTKAKRRVVEREKRAVGFGAFFLGFLGAAGSTMGAASITLTVQARQLLSGIVQQQNNLLRAIEAQQHLLKLTVWGIKQLQARVLAVERYLKDQQLLGIWGCSGKFICTTTVPWNSSWSNKSQSEIWDNMTWMQWDKEINNYTQIIYDLIEESQRQQEKNEQDLLALDKWANLWNWFDISNWLWYIKIFIMIVGGLIGLRIAFAVLSVINRVRQGYSPLSFQTHTPNPRDLDRPGRIEEEGGEQDRDRSIRLVSGFLALAWDDLRSLCLFSYHRLRDFILVAARTVELLGHISLKGLRRGWEGLKYLGNLLSYWGRELKISAINLLDTIAIVVAEWTDRIIEIGQRLCRAIINIPRRIRQGFERALL

SEQ ID NO: 3 - DNA, 2517 bp, synthetic construct
  misc_feature 1..2517: note = pGX1026 - Env Clade A tier 2 Q168ENVe2 DNA Sequence
  source 1..2517: mol_type = other DNA
atgaaggtgcgaggaatcaaaaggaatctgtggaaatgggggacaatgctgctgggaatgctgatgacatatagcgtggctgaacagctgtgggtgactgtctactatggcgtgccagtctggaaggacgctgaaaccacactgttctgcgcaagtgatgccaaggcttactcaaccgagaaacacaatatttgggctactcatgcatgcgtgcccaccgacccaaacccccaggaaatccacctggagaatgtgaccgaggagttcaacatgtggaaaaacaatatggtcgagcagatgcatacagacatcatttcactgtgggatcagagcctgcgaccatgcgtgaagctgacccctctgtgcgtcactctgaattgtaccaacgtgaacaacaacactaccaatgtcaacaacaacacagggtgggacgaggaaagaaagaactgttctttcaacatcacaactgagctgagggataagcgccagaaagtgtacagtctgttttataagctggacgtggtccagatcgataacagctcctaccggctgatcaattgcaacacatctgccattactcaggcttgtcctaaagtgaccttcgaacctatcccaattcactattgcgcaccagccggcttcgccatcctgaagtgtaaagatgagaagtttaatgggacaggaccctgcaaaaacgtgtctaccgtccagtgtacacatggaattaagcctgtggtctcaactcagctgctgctgaatggcagcctggctgaaaaagaagtgatgatccggagcgaaaatttcactaacaatgccaagaacattctggtgcagtttaaggagccagtcaaaatcaactgcaccagacccgacaacaataccagaacaagcatcaggattggccccgggcaggccttttacgctacaggcatcattggggatattaggcaggcatattgtactgtgaatggctccgagtggaacaaggccctgcagaaagtggtcgaacagctgcgctctagtttcgagaataagacaatcatcttcgccaactcaagcggcggggacctggaaatcaccacacacagtttcaattgcggaggcgagttcttttactgtaacacttccgggctgtttgattctacttggaatgacaccgatagcaggcaggagaacggaactatcaccctgccttgcagaattaagcagatcattaatatgtggcagaggaccggccaggcaatctatgcaccacctatccagggagcaattcgatgcgtgagcaacatcacaggactgattctgacccgggacgggggaaacaataacagcaccaatgaaacattcagaccaggcgggggagacatgcgcgataactggcgaagcgaactgtacaagtataaagtggtcaagatcgagcctctgggcgtggcaccaaccaaagcccggagaagggtggtcggacgagagaagcgagcagtgggaattggcgctgtcttcctgggatttctgggagcagctgggagcacaatgggagcagcctccatcacactgactgtgcaggccaggcagctgctgtctgggattgtccagcagcagagtaacctgctgaaagctatcgaagcacagcagcatctgctgcgcctgaccgtgtggggcatcaagcagctgcaggctagggtgctggcagtcgagcggtacctgaaagaccagcagctgctgggaatctggggctgctccgggaagctgatttgtactaccaatgtgccctggaactcctcttggtctaacaagagtcagtcagaaatctgggagaacatgacatggctgcagtgggaaaaggagattagcaattacacccagatcatctacacactgatcgaggaatcccagaatcagcaggagaagaacgagcaggacctgctggcactggataagtgggcctccctgtggaactggttcgatatctctaagtggctgtggtacatcaggatcttcatcatgattgtgggcgggctgatcggactgcgcatcgtgttcgccgtcctgagcgtggtcaaccgggtgagacagggctatagccctctgtcctttcagaccctgctgccagcacctcgggggccagacagacccgatggaattgaggaagagggaggagagcagggaaggggacgcagtcgacagctggtgaatggcttctcaacactgatctgggacgatctgcggaacctgtgcctgttttcctatcaccggctgagagacctgatcctgattgctgcaagaattgtggaactgctgggacgccgaggatgggaggctatcaaatacctgtggaacctgctgcagtattggattcaggagctgaagaattctgccattagtctgctgaacacaactgctatcgcagtggccgaaggcaccgatcgagccatcgagatcattcagcgggctattaccgccgtcctgaacattcctacccgcattagacagggatttgaacgcgctctgctgtgataa

SEQ ID NO: 4 - protein, 837 aa, synthetic construct
  REGION 1..837: note = pGX1026 - Env Clade A tier 2 Q168ENVe2 DNA Sequence
MKVRGIKRNLWKWGTMLLGMLMTYSVAEQLWVTVYYGVPVWKDAETTLFCASDAKAYSTEKHNIWATHACVPTDPNPQEIHLENVTEEFNMWKNNMVEQMHTDIISLWDQSLRPCVKLTPLCVTLNCTNVNNNTTNVNNNTGWDEERKNCSFNITTELRDKRQKVYSLFYKLDVVQIDNSSYRLINCNTSAITQACPKVTFEPIPIHYCAPAGFAILKCKDEKFNGTGPCKNVSTVQCTHGIKPVVSTQLLLNGSLAEKEVMIRSENFTNNAKNILVQFKEPVKINCTRPDNNTRTSIRIGPGQAFYATGIIGDIRQAYCTVNGSEWNKALQKVVEQLRSSFENKTIIFANSSGGDLEITTHSFNCGGEFFYCNTSGLFDSTWNDTDSRQENGTITLPCRIKQIINMWQRTGQAIYAPPIQGAIRCVSNITGLILTRDGGNNNSTNETFRPGGGDMRDNWRSELYKYKVVKIEPLGVAPTKARRRVVGREKRAVGIGAVFLGFLGAAGSTMGAASITLTVQARQLLSGIVQQQSNLLKAIEAQQHLLRLTVWGIKQLQARVLAVERYLKDQQLLGIWGCSGKLICTTNVPWNSSWSNKSQSEIWENMTWLQWEKEISNYTQIIYTLIEESQNQQEKNEQDLLALDKWASLWNWFDISKWLWYIRIFIMIVGGLIGLRIVFAVLSVVNRVRQGYSPLSFQTLLPAPRGPDRPDGIEEEGGEQGRGRSRQLVNGFSTLIWDDLRNLCLFSYHRLRDLILIAARIVELLGRRGWEAIKYLWNLLQYWIQELKNSAISLLNTTAIAVAEGTDRAIEIIQRAITAVLNIPTRIRQGFERALL

SEQ ID NO: 5 - DNA, 2562 bp, synthetic construct
  misc_feature 1..2562: note = pGX1027 - Env Clade A tier 2 Q842ENVd12 DNA Sequence
  source 1..2562: mol_type = other DNA
atgagagcgatggggatacagatgaattgtcaaaacttgtggaggtgggggactatgatcttggggatgataatattctgtagtgctgtagacaacttgtgggttactgtctactatggggtacctgtgtggaaagaggcagaaaccaccttattttgtgcatcagatgctaaagcatatgagacagaaaaacataatgtctgggctacacatgcctgtgtacccacagaccccaacccacaagaaatacatttggaaaatgtgacagaagagtttaacatgtggaaaaataacatggtagagcagatgcatacagatataatcagtctatgggaccaaagcctaaagccatgtgtaaagttaacccctctctgtgttactttagattgtaacaatgtcaccaataatggcaccagtgacatgagagaagaaataaaaaactgctctttcaatatgaccacagaactaagggataagagacagaaagtatattcacttttttataaacttgatatagtacaaattaatgaagatcagggtaatagtagtaacaataagtatagattaataacttgtaatacctcagccattacacaagcatgcccaaaggtaacctttgagccaattcccatacattattgtgctccagctggttttgcgatcctaaagtgtaaggatgaggagttcaatggaatagggccatgcaagaatgtcagcacagtccaatgcacacatggaatcaagccagtagtatcaactcaactactgttaaatggcagtctagcagaaaaagaggtaaaaattagatgtgaaaatatcacaaacaatgctaaaactataatagtacaacttgtcaatcctgtgaaaattaattgtaccagacctaacaacaatacaagaaaaagtatacatataggaccaggacaagcattctatgcaacaggtgacataataggggatataagacaagcacattgtaatgtcaacaggacagaatggaacaacactttgcaccaggtagtcgaacaattaagaaaacactttaacaaaacaataaactttgctaactccacaggaggggatctagaaataacaacacatagttttaattgtggaggagaatttttctattgcaatacaacaaacctgtttaatagcacttggaatcacactgccagcatgaatagcacagagtcaaatgacactataattctcccatgcagaataaaacaaattataaatatgtggcagagagtaggacaagcaatgtatgcccctcccattcgaggagtaataaggtgtgaatcaaacattacaggactaatattaacaagagatggtgggaatactaacagtacaagggaaaccttcagacctggaggtggagatatgagggacaattggagaagtgaattatacaagtataaagtagtaaaaattgaaccactaggagtagcacccaccaaggcaaagagaagagtggtggagagagaaaaaagagcagttggaataggagctgtcttcattgggttcttaggagcagcgggaagcactatgggcgcggcgtcaataacgctgacggtacaggccagacaattattgtctggcatagtgcaacagcaaagcaatttgctgagggctatagaggctcaacagcatctgttgaaactcacggtctggggcattaaacagctccaggcaagagtcctggctgtggaaagatacctaaaggatcaacagctcctaggaatttggggctgctctggaaaactcatctgcaccactagtgtgccctggaattctagttggagtaataaatcccagaatgagatatgggacaacatgacctggctgcaatgggataaagaaattagcaattacacacagataatatatgatctacttgaagaatcgcagaaccagcaggaaaagaatgaacaagacttattggcattggacaagtgggcaaatctgtggaattggtttgacatatcaaactggctgtggtatataaaaatatttataatgatagtaggaggtttaataggattaagaatagtttttgctgtgctttctgtaataaatagagttaggcagggatactcacctttgtcgttccagacccataccccaaacccaaggggtctcgacaggcccgaaagaatcgaagaagaaggtggagagcaagacaaaaacagatcgattcgattagtgagcggattcttagcacttgcctgggacgatctacggagcctgtgcctcttcagctaccaccgattgagagacttcatcttgattgtagcgaggactgtggaacttctgggacacagcagtctcaaggggctgagactggggtgggaaggcctcaagtatctggggaatcttctatcatattggggtcgggaactaaggattagtgctactaatttgcttgataccatagcaatagtaatagctgggtggacagatagggttatagaaataggacagagactttgtagagcttttctcaacatacctagaagaatcagacagggcttcgaaagggctttgctatgataa

SEQ ID NO: 6 - protein, 852 aa, synthetic construct
  REGION 1..852: note = pGX1027 - Env Clade A tier 2 Q842ENVd12 Amino Acid Sequence
MRAMGIQMNCQNLWRWGTMILGMIIFCSAVDNLWVTVYYGVPVWKEAETTLFCASDAKAYETEKHNVWATHACVPTDPNPQEIHLENVTEEFNMWKNNMVEQMHTDIISLWDQSLKPCVKLTPLCVTLDCNNVTNNGTSDMREEIKNCSFNMTTELRDKRQKVYSLFYKLDIVQINEDQGNSSNNKYRLITCNTSAITQACPKVTFEPIPIHYCAPAGFAILKCKDEEFNGIGPCKNVSTVQCTHGIKPVVSTQLLLNGSLAEKEVKIRCENITNNAKTIIVQLVNPVKINCTRPNNNTRKSIHIGPGQAFYATGDIIGDIRQAHCNVNRTEWNNTLHQVVEQLRKHFNKTINFANSTGGDLEITTHSFNCGGEFFYCNTTNLFNSTWNHTASMNSTESNDTIILPCRIKQIINMWQRVGQAMYAPPIRGVIRCESNITGLILTRDGGNTNSTRETFRPGGGDMRDNWRSELYKYKVVKIEPLGVAPTKAKRRVVEREKRAVGIGAVFIGFLGAAGSTMGAASITLTVQARQLLSGIVQQQSNLLRAIEAQQHLLKLTVWGIKQLQARVLAVERYLKDQQLLGIWGCSGKLICTTSVPWNSSWSNKSQNEIWDNMTWLQWDKEISNYTQIIYDLLEESQNQQEKNEQDLLALDKWANLWNWFDISNWLWYIKIFIMIVGGLIGLRIVFAVLSVINRVRQGYSPLSFQTHTPNPRGLDRPERIEEEGGEQDKNRSIRLVSGFLALAWDDLRSLCLFSYHRLRDFILIVARTVELLGHSSLKGLRLGWEGLKYLGNLLSYWGRELRISATNLLDTIAIVIAGWTDRVIEIGQRLCRAFLNIPRRIRQGFERALL

SEQ ID NO: 7 - DNA, 2601 bp, synthetic construct
  misc_feature 1..2601: note = pGX1028 - Env Clade A tier 2 Q461ENVe2 DNA Sequence
  source 1..2601: mol_type = other DNA
atgagagtgatggggattcagaggaactatcagcacctgtggagatgggggacaatgctgctgggaatgctgatgacctgtagcgtcacaggacagtgggtgactgtctactatggcgtgcccgtctggaaggacgcagagaccacactgttctgcgcctctgatgctaaggcatacgagacagaaaaacacaacgtgtgggctacacatgcatgcgtgcctactgacccaaacccccaggagatcaggctggaaaatgtgaccgaggacttcaacatgtggaagaatagcatggtggaacagatgaatgaggacatcatttctctgtgggatcagagtctgaagccatgcgtgaaactgacccctctgtgcgtgaccctgaactgtaccgactggacaaacaatgctacatcaactaatcagactacccccgcaactagcgaggaaaccggcgtgaagaactgttccttcaatattacaactgagctgagggacaagaaacagaaggtgtactccctgttttataaactggatgtggtccagatctctgaaaacaatagctccaactctagtaatttcacccagtaccgcctgattaactgcaatacatcagccatcactcaggcttgtcccaaggtgagctttgagcctatcccaattcactattgcgcccctgctggcttcgccattctgaaatgtaacgatagcgtgttcaacggcaccgggccatgcaagaacgtgtcaaccgtccagtgtacacatggcatcaaacccgtggtctcaacacagctgatgctgaatgggagcctggcagaacgcaaagtgatgattcgaagcgagaacatcactaacaatgccaagaatatcattgtgcagttcaccaaacctgtcaacattacatgcatcaggccaggcaacaatacccgaaaatccgtgcggatcggaccaggccaggccttttacgctactggcgacattaccggggatatccgaaacgctcactgcgtggtcaatcggactgagtggaacaataccctgcagaaggtggtcgaacagctgcgcgagtacttccccaacaaaacaatcatcttcaccaattcaagcggcggggacatcgaaattaccacacatagcttcaattgcggaggcgagttcttttattgtaacacctcaaagctgtttaatagccggtgggagaacaatgggactgccaacatgctgaaaaatgataccggcagcaacgaaactaccctgattctgaggtgccgcatcaagcagatcattaatatgtggcagagagtgggccaggcaatgtatgcccctcccattcagggcgtgatcaactgtacctctaatattacaggactgatcctgacaagagacgggggaggcgaaaacgataccgagacattcaggcctgggggaggcgacatgagagataattggaggagcgaactgtacaagtataaagtggtcaagctggagccactgggagtggcacctaccatggccaagcggagagtggtcgagcgggaaaaaagagcagtgggaatggcagctgtcttcctggggtttctgggaactgctggcagcaccatgggagcagcatccctgactctgaccgtgcaggcacgacagctgctgtctggcattgtccagcagcagagtaacctgctgaaggctatcgaggcacagcagcacctgctgagactgaccgtgtggggcatcaaacagctgcaggctcgggtgctggcagtcgagagatacctgaaggaccagcagctgctggggatttggggatgctccggcaaactgatctgtacaacttctgtgccctggaactcctcttggagtaataagacccagcaggaaatctggaacaataccacatggctgcagtgggacaaagagattagcaactacacaggcactatctatcggctgctggaggaatcccagaaccagcaggagaagaatgaacaggacctgctggccctggataaatgggctaacctgtggaattggttcgatatctctaagtggctgtggtacatcaaaatcttcatcatggtggtcgggggactgattgggctgagaatcgtgttcgccatcattagtgtggtcaaccgagtgcggcagggatatagccctctgtcctttcagatccccacacctaatccagaaggactggacaggccaggacgaattgaggaaggcgggggagagcaggatagaaccaggtccatccgcctggtgtctggcttcctggcactggcctgggacgatctgcgaagtctgtgcctgttctcatatcaccgcctgcgagactttattctgatcgtggccaggaccgtcgaactgctggggcatagttcactgaagggactgcgcctggggtgggagggactgaaatacctgggcaacctgctgtcttattgggggcaggaactgaagaacagtgctacaaatctgctggacactaccgctattgcagtggccggctggactgatagggccattgagatcgtgcagcgcatcgtcagagccattctgcatattccacgccgcattagacagggatttgaacgcgcactgctgtaa

SEQ ID NO: 8 - protein, 866 aa, synthetic construct
  REGION 1..866: note = pGX1028 - Env Clade A tier 2 Q461ENVe2 Amino Acid Sequence
MRVMGIQRNYQHLWRWGTMLLGMLMTCSVTGQWVTVYYGVPVWKDAETTLFCASDAKAYETEKHNVWATHACVPTDPNPQEIRLENVTEDFNMWKNSMVEQMNEDIISLWDQSLKPCVKLTPLCVTLNCTDWTNNATSTNQTTPATSEETGVKNCSFNITTELRDKKQKVYSLFYKLDVVQISENNSSNSSNFTQYRLINCNTSAITQACPKVSFEPIPIHYCAPAGFAILKCNDSVFNGTGPCKNVSTVQCTHGIKPVVSTQLMLNGSLAERKVMIRSENITNNAKNIIVQFTKPVNITCIRPGNNTRKSVRIGPGQAFYATGDITGDIRNAHCVVNRTEWNNTLQKVVEQLREYFPNKTIIFTNSSGGDIEITTHSFNCGGEFFYCNTSKLFNSRWENNGTANMLKNDTGSNETTLILRCRIKQIINMWQRVGQAMYAPPIQGVINCTSNITGLILTRDGGGENDTETFRPGGGDMRDNWRSELYKYKVVKLEPLGVAPTMAKRRVVEREKRAVGMAAVFLGFLGTAGSTMGAASLTLTVQARQLLSGIVQQQSNLLKAIEAQQHLLRLTVWGIKQLQARVLAVERYLKDQQLLGIWGCSGKLICTTSVPWNSSWSNKTQQEIWNNTTWLQWDKEISNYTGTIYRLLEESQNQQEKNEQDLLALDKWANLWNWFDISKWLWYIKIFIMVVGGLIGLRIVFAIISVVNRVRQGYSPLSFQIPTPNPEGLDRPGRIEEGGGEQDRTRSIRLVSGFLALAWDDLRSLCLFSYHRLRDFILIVARTVELLGHSSLKGLRLGWEGLKYLGNLLSYWGQELKNSATNLLDTTAIAVAGWTDRAIEIVQRIVRAILHIPRRIRQGFERALL

SEQ ID NO: 9 - DNA, 2532 bp, synthetic construct
  misc_feature 1..2532: note = pGX1039 - Env Clade A tier 2 Q259d2.17 DNA Sequence
  source 1..2532: mol_type = other DNA
atgaactcacagaactcactgcgatggggcattactatcctgggcatgattattatttgctctgctgctgaaaacctgtgggtcaccgtgtactatggggtgcctgtctggaaagacgccgagaccacactgttctgcgcttctaatgccaaggcttacggaaccgaagtcgagaacatctgggcaacccacgcctgcgtgccaacagatccaaatccccaggaaattaatctggagaacgtcactgaggagttcaacatgtggaagaacaatatggtggaacagatgcataccgacatcattagcctgtgggatcagggcctgaaaccttgcgtgaagctgactccactgtgcgtcaccctggactgttataatgtgactaagtcagacaaaatcaccaaggatatgcaggaggaaatcaaaaactgtagcttcaacatcactaccgagctgcgcgataagaaacagaaggtgcacagcctgttttaccgactggacgtggtccccatgggcgggaaaaacgatagtcagtataggctgatcaattgcaacacttcagcaattacccaggcctgtcccaaggtgacattcgagcctatcccaattcactactgcgcacctgccggcttcgccatcctgaaatgtaatgacaaggaattttctggcactgggccatgcaagaacgtgagctccgtccagtgtacccatggaatcaggcccgtggtctccacacagctgctgctgaacggctctctggccgaggaaaaggtgcggatcagaagcgaggatatcacaaacaacggcaaaaacatcatcgtgcagctgaagactccagtcaacatcagctgcacacgccccaacaataacactagaaagtccgtgaggattggacccggccaggctttttatgcaaccgacgatatcattgggaatatccgacaggcctactgtacagtcaaccggactcagtgggactataccctgcaggaggtggctaatcagctgagaatctacttcaacaaaacaatcatcttcaacaactctgccggaggcgacctggaaattacaactcacagtttcaattgcgggggagagttcttttattgtgatacctcagggctgtttaatagcacttggacctggaacgacaccgtgagctggcaaggaagtgataatatcaccctgcagtgcagaattaagcagatcattaacatgtggcagagggccggacaggctatctacgcaccccctatccagggcgtgattaggtgtgacagcaacatcacagggctgattctgactcgcgatggcggaaataactctagtcccaatgagatcttccggcctggaggcggggacatgcgagataactggcgatccgaactgtacaagtataaagtggtcaagatcgagccactgggcgtggctcccacaagagcaaaacggagagtggtcgaacgggagaagagagcagtggggatcggagccgtcttcattggctttctgggagcagctggatctaccatgggagcagccagtatcacactgactgctcaggcaaggaagctgctgtcagggatcgtccagcagcagagcaacctgctgcgcgccattgaggctcagcagcatctgctgaaactgaccgtgtggggcatcaagcagctgcaggcccgggtgctggctgtcgaaagatacctgaaagaccagcagctgctgggaatctggggatgctccggaaagctgatttgtaccacaaatgtgccctggaactcaagctggtctaataagagtcagtcagaaatctgggagaacatgacctggctgcagtgggacaaagaaattaataactacacacagctgatctattccctgattgagaagtctcagactcagcaggaaatcaatgagcaggacctgctggctctggataaatgggcaaatctgtggaactggttcgatatttccaactggctgtggtacatccggatcttcatcatgattgtcggaggcctgatcggactgagaatcgtgttcgccgtcctgagtatcattaaccgagtgcggcagggacacagccctctgtcctttcagacccatacaccaagccctcgggaactggacaggcctggacgaatcgaggaagagggcggcgagccagatagaggcaggagtattaggctggtgtcagggttcctggccctggcttgggacgatctgcgcagcctgtgcctgttctcctatcaccgcctgcgagactttatcagcattgctgcacggacagtggaactgctgggacattcctctctgaaaggcctgagactgggctgggaggggctgaagtacctggggaatctgctggtgtattggggacgagaactgcggctgtccgccatcaacctgctggataccatcgcaattgccaccgctgactggacagatagagtgatcgagctgggccagcgcctgtgccgagctattctgcatattcccaggaggattcgccagggatttgagagagcactgctgtgataa

SEQ ID NO: 10 - protein, 842 aa, synthetic construct
  REGION 1..842: note = pGX1039 - Env Clade A tier 2 Q259d2.17 Amino Acid Sequence
MNSQNSLRWGITILGMIIICSAAENLWVTVYYGVPVWKDAETTLFCASNAKAYGTEVENIWATHACVPTDPNPQEINLENVTEEFNMWKNNMVEQMHTDIISLWDQGLKPCVKLTPLCVTLDCYNVTKSDKITKDMQEEIKNCSFNITTELRDKKQKVHSLFYRLDVVPMGGKNDSQYRLINCNTSAITQACPKVTFEPIPIHYCAPAGFAILKCNDKEFSGTGPCKNVSSVQCTHGIRPVVSTQLLLNGSLAEEKVRIRSEDITNNGKNIIVQLKTPVNISCTRPNNNTRKSVRIGPGQAFYATDDIIGNIRQAYCTVNRTQWDYTLQEVANQLRIYFNKTIIFNNSAGGDLEITTHSFNCGGEFFYCDTSGLFNSTWTWNDTVSWQGSDNITLQCRIKQIINMWQRAGQAIYAPPIQGVIRCDSNITGLILTRDGGNNSSPNEIFRPGGGDMRDNWRSELYKYKVVKIEPLGVAPTRAKRRVVEREKRAVGIGAVFIGFLGAAGSTMGAASITLTAQARKLLSGIVQQQSNLLRAIEAQQHLLKLTVWGIKQLQARVLAVERYLKDQQLLGIWGCSGKLICTTNVPWNSSWSNKSQSEIWENMTWLQWDKEINNYTQLIYSLIEKSQTQQEINEQDLLALDKWANLWNWFDISNWLWYIRIFIMIVGGLIGLRIVFAVLSIINRVRQGHSPLSFQTHTPSPRELDRPGRIEEEGGEPDRGRSIRLVSGFLALAWDDLRSLCLFSYHRLRDFISIAARTVELLGHSSLKGLRLGWEGLKYLGNLLVYWGRELRLSAINLLDTIAIATADWTDRVIELGQRLCRAILHIPRRIRQGFERALL

SEQ ID NO: 11 - DNA, 2553 bp, synthetic construct
  misc_feature 1..2553: note = pGX1030 - Env Clade B tier 2 WITO4160.33 DNA Sequence
  source 1..2553: mol_type = other DNA
atgaaagtgatgggaacaaagaagaactaccagcacctgtggagatgggggattatgctgctgggaatgctgatgatgtcaagcgcagccgagcagctgtgggtgaccgtctactatggggtgccagtctggagagaagcaaacaccacactgttctgcgccagcgacgctaaagcatacgatacagaggtgcacaatgtctgggcaacccatgcctgcgtgcccacagacccaaacccccaggaggtggtcatgggcaatgtgaccgaagacttcaacatgtggaagaacaatatggtggagcagatgcacgaagacatcatttccctgtgggatcagtctctgaagccctgcgtcaaactgacacctctgtgcgtgactctgcattgtacaaacgtcactatcagctccaccaatggcagcacagctaacgtgactatgagggaggaaatgaagaattgttccttcaacactaccacagtgattcgcgacaagatccagaaagagtacgcactgttttataaactggatattgtgccaatcgaaggcaagaacactaataccgggtacagactgattaactgcaataccagtgtgatcacacaggcctgtcctaaggtgtcattcgagcctattccaatccactattgcgccccagctggcttcgctattctgaagtgtaacaacaagaccttcaacgggaaaggaccctgcaggaacgtgagcactgtccagtgtacccatgggatcaagcctgtggtctccacccagctgctgctgaacggatctctggccgaggaagacatcattatccgctccgagaatttcacaaacaacgggaaaaacatcatcgtccagctgaaggaaccagtgaaaatcaattgcactcggcccggaaacaatacccggagaagtattaacatcggccctgggcgcgctttttacgcaaccggggccattatcggagatattcgaaaggcccactgtaatatcagcacagagcagtggaacaatacactgactcagatcgtggacaaactgcgcgaacagttcggaaataagactatcatctttaaccagtctagtggcggcgaccccgaggtggtcatgcatacattcaactgcggaggcgaattcttttactgtaatagcacacagctgttcaactccacttggtttaacaatggcacctcaacatggaatagcaccgccgacaacatcacactgccatgccggatcaagcaggtcatcaacatgtggcaggaggtcgggaaggctatgtatgcaccccctattcgcggacagatcgactgttcaagcaacattactggactgatcctgacccgggatggaggcagcaattcctctcagaacgagacctttagacccggcgggggaaatatgaaagataactggaggtctgagctgtacaagtataaagtggtcaagattgaacctctgggcatcgcaccaacaagagccaaaaggcgagtggtccagcgagagaagcgagcagtgactctgggagctgtcttcctgggatttctgggagcagctgggtctaccatgggagcagccagtctgactctgaccgtgcaggcccgactgctgctgtcaggcattgtgcagcagcagagcaatctgctgagggccatcgaggctcagcagcacatgctgcagctgaccgtgtggggcatcaagcagctgcaggctagggtgctggcaatcgaacgctacctgaaagaccagcagctgctgggaatttggggctgctctgggaagctgatctgtactaccacagtgccctggaatacaagttggtcaaacaagagttacgactatatttggaacaatatgacttggatgcagtgggagagggaaatcgataactacacaggcttcatctacactctgatcgaggaatcacagaatcagcaggagaaaaacgagctggaactgctggaactggataagtgggccagcctgtggaactggttcaatatcaccaactggctgtggtacattaagctgtttatcatgattatcggcgggctggtgggactgagaatcgtgtgcgctgtcctgtctatcgtgaatagagtcaggcagggctatagccctctgtcctttcagactaggctgcccaaccctcggggaccagacagacccgaggaaaccgagggagaaggaggagagcgagaccgagatcggtccgctcgactggtgaatggcttcctggcaattatctgggacgatctgagaagtctgtgcctgttttcatatcatagactgagggatctgctgctgattgtggcccgggtggtcgagatcctgggacgacggggctgggaaatcctgaagtactggtggaacctgctgaaatattggagccaggagctgaagaattctgcagtgagtctgctgaacgtcaccgcaatcgccgtggctgagggcacagaccgagtgattgaaatcgtccagcgggccgtgagagccattctgcatattcccacccgcattcgccagggatttgaacgcgcactgctgtgataa

SEQ ID NO: 12 - protein, 849 aa, synthetic construct
  REGION 1..849: note = pGX1030 - Env Clade B tier 2 WITO4160.33 Amino Acid Sequence
MKVMGTKKNYQHLWRWGIMLLGMLMMSSAAEQLWVTVYYGVPVWREANTTLFCASDAKAYDTEVHNVWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTGYRLINCNTSVITQACPKVSFEPIPIHYCAPAGFAILKCNNKTFNGKGPCRNVSTVQCTHGIKPVVSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIMIIGGLVGLRIVCAVLSIVNRVRQGYSPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 13 - DNA, 2577 bp, synthetic construct
  misc_feature 1..2577: note = pGX1033 - Env Clade B tier 3 TRJO4551.58 DNA Sequence
  source 1..2577: mol_type = other DNA
atgcgcgtgatggggattaggaaaaactaccagcacctgtggagatggggcactatggggatgatgctgctggggattctgatgatttgcaacgccacagaaaaactgtgggtgactgtctactatggcgtgccagtctggaaggaggctaccacaactctgttctgcgcaagcgatgccaaagcttacgagacagaagtgcacaatgtctgggcaacccatgcctgcgtgcccacagatccaaacccccaggagctggtgctggaaaatgtcactgagtattttgacatgtggaagaacaatatggtggaacagatgcacgaggacatcatttccctgtgggatcagtctctgaaaccctgcgtgaagctgactcctctgtgcgtcaccctgaactgtaccgactggacaaatggcactgattggaacaccacaaactctaacaacactaccatcagtaaggaggaaactattgagggcggggaaatgaagaactgtagcttcaatatcacaactgccaccggggacaagaaaaaggaaagggcattcttttacaagctggacgtggcccccatcgataactcaaataccagctatcgcctgatctcttgcaacaccagtgtgattacacaggcatgtcccaaaatcagctttgagcctatcccaattcactactgcgcacctgccggcttcgctatcctgaagtgtaacgataagaagtttaatggaactggcagttgcaccaacgtgtcaacagtccagtgtactcatggaattcggcctgtggtctccacccagctgctgctgaatggctctctggctgaggaagaggtggtcatcagatcaaaaaacttcagcgacaatgcaaagatcattatcgtgcagctgaacgagtctgtcccaatcaattgcactcgaccccacaacaatacccggaaaagtatccatattgggccaggacgagcttggtacgcaaccggggacattatcggagatattagaaaggcctattgtaacatctccgaggctaaatggaacaatacactgaagcagatcactgaaaaactgaaggagcagttcaacaagactattatcgtgtttaatcagccaagcggaggcgatcccgaagtgaccatgcactccttcaactgcgggggagagttcttttactgtaacaccagtaagctgtttaacgggacctggaattcaacaaagagggccaacaatacagagggaattatcattctgcagtgcagaatcaaacagatcattaacaggtggcaggaagtgggaaaggccatgtatgctccccctatcgagggccagattaagtgtagctccaatatcaccgggctgctgctgacaagggatggcgggaaaaccgccaacaataccacagagttctttcgccccggaggcgggaacatgaaagacaattggaggagcgaactgtacaaatataaggtggtccgcatcgagcctctgggagtggctccaacaaaagcaaagcggagagtggtccagcgcgagaagcgagcaatcggcattggggccgtgttcctgggatttctgggagcagctgggtcaaccatgggagcagccagcatcacactgactgtgcaggcccggaaactgctgtccggcattgtgcagcagcagaacaatctgctgagagcaatcgaagcccagcagcacctgctgcagctgaccgtgtggggcatcaagcagctgcaggcccgggtgctggctgtcgagcggtacctgagagaccagcagctgctgggaatttggggctgctctgggaagctgatctgtactaccgccgtgccctggaactctagttggtccaacaagtctctggatacaatttggaacaatatgacttggatgcagtgggagaaggaaatcgacaactacactggcctgatctataccctgattgaagagtcacagattcagcaggagaaaaatgaactggacctgctgaagctggatcagtgggccagcctgtggaactggttcgatatcacaaattggctgtggtacatcaagatcttcatcatgattgtgggaggactggtcggactgcgaatcgtgttcgctgtcctgtccatcgtgaaccgagtccggcagggctatagtcctctgtcatttcagacccatctgccaaattctagggggccagaccgacctggaggaatcgaagaggaaggcggggagagggacaacggcagaagtaggcctctggtggatgggttcctggccatcatttgggtcgacctgcgcagcctgtgcctgttttcctatcaccatctgcggggcctgctgctgatcgctgcaagaattgtggaactgctgggaaggcgcggatgggaggccctgaagtactggtggaacctgctgcagtattgggggcaggagctgagaaacagcgccgtgagcctgctgaatgctaccgcaattgccgtggctgaaggaacagaccgcatcattgaggtggtccagcgaatcggccgagccattctgaacatcccccgacgcattagacagggagccgaaagagcactgcagtgataa

SEQ ID NO: 14 - protein, 857 aa, synthetic construct
  REGION 1..857: note = pGX1033 - Env Clade B tier 3 TRJO4551.58 Amino Acid Sequence
MRVMGIRKNYQHLWRWGTMGMMLLGILMICNATEKLWVTVYYGVPVWKEATTTLFCASDAKAYETEVHNVWATHACVPTDPNPQELVLENVTEYFDMWKNNMVEQMHEDIISLWDQSLKPCVKLTPLCVTLNCTDWTNGTDWNTTNSNNTTISKEETIEGGEMKNCSFNITTATGDKKKERAFFYKLDVAPIDNSNTSYRLISCNTSVITQACPKISFEPIPIHYCAPAGFAILKCNDKKFNGTGSCTNVSTVQCTHGIRPVVSTQLLLNGSLAEEEVVIRSKNFSDNAKIIIVQLNESVPINCTRPHNNTRKSIHIGPGRAWYATGDIIGDIRKAYCNISEAKWNNTLKQITEKLKEQFNKTIIVFNQPSGGDPEVTMHSFNCGGEFFYCNTSKLFNGTWNSTKRANNTEGIIILQCRIKQIINRWQEVGKAMYAPPIEGQIKCSSNITGLLLTRDGGKTANNTTEFFRPGGGNMKDNWRSELYKYKVVRIEPLGVAPTKAKRRVVQREKRAIGIGAVFLGFLGAAGSTMGAASITLTVQARKLLSGIVQQQNNLLRAIEAQQHLLQLTVWGIKQLQARVLAVERYLRDQQLLGIWGCSGKLICTTAVPWNSSWSNKSLDTIWNNMTWMQWEKEIDNYTGLIYTLIEESQIQQEKNELDLLKLDQWASLWNWFDITNWLWYIKIFIMIVGGLVGLRIVFAVLSIVNRVRQGYSPLSFQTHLPNSRGPDRPGGIEEEGGERDNGRSRPLVDGFLAIIWVDLRSLCLFSYHHLRGLLLIAARIVELLGRRGWEALKYWWNLLQYWGQELRNSAVSLLNATAIAVAEGTDRIIEVVQRIGRAILNIPRRIRQGAERALQ

SEQ ID NO: 15 - DNA, 2604 bp, synthetic construct
  misc_feature 1..2604: note = pGX1037 - Env Clade B tier 3 PVO.4 DNA Sequence
  source 1..2604: mol_type = other DNA
atgagggtcacagggattagaaaaaactaccagcactcttggagatggggaatgatgctgctgggaatgctgatgatttgttctgccgaggaaaagctgtgggtgacagtctactatggcgtgcctgtctggaaagaagcaaccacaactctgttctgcgcctccgacgctaaggcatacaatactgaggtgcacaacgtctgggctactcatgcatgcgtgccaaccgatccaaatccccaggaagtggggctggaaaacgtcaccgagaactttaatatgtggaagaacaatatggtggaacagatgcacgaggacatcatttcactgtgggatcagagcctgaagccctgcgtgaaactgacacctctgtgcgtcactctgaactgtagcgacctgcggaacgccacaaataccacaaaccctactgtgagctccagagtcattaagaaagaaatgatgggcgaggtgaaaaattgctccttcaacgtcactaccgacatccgggatagaatgcagaaggtgtacgccctgttttataggccagacgtggtccccatccaggatcataccatcgaaaacaacaacacaatcgagaacaacacaacttaccgcctgatctcttgcaatacaagtgtgattactcaggcttgtcccaaaatcagcttcgagcctatcccaattcactattgcacacctgccggcttcgctattctgaagtgtaacgataagaagttcaacggctctgggccatgcaccaacgtgagtacagtccagtgtactcatggcatcaggcccgtggtctcaacccagctgctgctgaatgggagccgagccgaggaagaagtgatcattcggagcgaaaacttcaccaataacgctaagacaatcattgtgcagctgaacaagactgtcgagatcaactgcacccgccctaataacaatacacgaaagtcaatcagcattggaccaggcagggccttctacgccaccggagacatcattggcgatattagacaggctcactgtaatctgtccagggcagaatggaacaagactctgaaatatatctctaccaagctgcgcgagcagttcgggaacaagaccatcatcttcaacggatctagtggcggggaccccgaaatcgtgacacatagcttcaactgcggaggcgagttcttttactgtaataccacaaagctgtttaacagtacctgggatgccaacgggaattgcacaggatgtgacgaatcagatggcaacaatacaatcactctgccttgcagaatcaagcagattgtgaatatgtggcaggaggtcggcaaagctatgtatgcaccccctatcaaggggctgatcaagtgtacctctaacatcacaggactgctgctgacaagggacgggggagccaacaatactaatgagaccttccgcccaggaggaggagacatgcgagataactggcggagtgaactgtacaagtataaagtggtccagatcgagcctctgggaattgcaccaacccgggcccggagaagggtggtccagagggagaagcgagcagtggggactctgggagctatgttcctgggctttctgggggccgctggaagtaccatgggagcagcctcagtgaccctgacagtccaggccagacagctgctgtccggcattgtgcagcagcagaacaatctgctgaaagccatcgaagctcagcagcacatgctgcagctgacagtgtggggcattaagcagctgcaggctcgggtgctggcaatcgagagatacctgaaagatcagcagctgctgggcatttgggggtgcagcggaaagctgatctgtactaccgccgtgccatggaatacctcctggtctaataagtccttcaacaaaatctgggacaacatgacatggatggaatgggagagggaaattgataattacactggcctgatctataacctgctggaagagtctcagaatcagcaggagaagaacgaacaggacctgctggctctggataaatgggagagcctgtggaattggttctccattaccaagtggctgtggtacatcaaaatcttcatcatgattgtgggaggactgatcggactgcgaatcgtgttcgcagtcctgtctatcgtgaacagggtccgccagggatatagtccactgtcatttcagactcacctgcccaccagtagaggaccagacaggcctgagggaatcggaggagagggaggagaacgagaccgagatagatcaggccccctggtggacgggtttctggccatcatttgggtggatctgcgctccctgttcctgttttcttatcatcgactgacagatctgctgctgatcctgactcggattgtggaactgctgggccgccgaggatgggaggcactgaagtactggtggaacctgctgcagtattggagccaggagctgagaaatagcgccgtgtccctgctgaacgccactgctatcgcagtggccgaaggcaccgacaggatcattgaggtggtccagcgcaccttccgcgccattattcatattccaagacgcattagacagggactggagagactgctgctgtgataa

SEQ ID NO: 16 - protein, 866 aa, synthetic construct
  REGION 1..866: note = pGX1037 - Env Clade B tier 3 PVO.4 Amino Acid Sequence
MRVTGIRKNYQHSWRWGMMLLGMLMICSAEEKLWVTVYYGVPVWKEATTTLFCASDAKAYNTEVHNVWATHACVPTDPNPQEVGLENVTENFNMWKNNMVEQMHEDIISLWDQSLKPCVKLTPLCVTLNCSDLRNATNTTNPTVSSRVIKKEMMGEVKNCSFNVTTDIRDRMQKVYALFYRPDVVPIQDHTIENNNTIENNTTYRLISCNTSVITQACPKISFEPIPIHYCTPAGFAILKCNDKKFNGSGPCTNVSTVQCTHGIRPVVSTQLLLNGSRAEEEVIIRSENFTNNAKTIIVQLNKTVEINCTRPNNNTRKSISIGPGRAFYATGDIIGDIRQAHCNLSRAEWNKTLKYISTKLREQFGNKTIIFNGSSGGDPEIVTHSFNCGGEFFYCNTTKLFNSTWDANGNCTGCDESDGNNTITLPCRIKQIVNMWQEVGKAMYAPPIKGLIKCTSNITGLLLTRDGGANNTNETFRPGGGDMRDNWRSELYKYKVVQIEPLGIAPTRARRRVVQREKRAVGTLGAMFLGFLGAAGSTMGAASVTLTVQARQLLSGIVQQQNNLLKAIEAQQHMLQLTVWGIKQLQARVLAIERYLKDQQLLGIWGCSGKLICTTAVPWNTSWSNKSFNKIWDNMTWMEWEREIDNYTGLIYNLLEESQNQQEKNEQDLLALDKWESLWNWFSITKWLWYIKIFIMIVGGLIGLRIVFAVLSIVNRVRQGYSPLSFQTHLPTSRGPDRPEGIGGEGGERDRDRSGPLVDGFLAIIWVDLRSLFLFSYHRLTDLLLILTRIVELLGRRGWEALKYWWNLLQYWSQELRNSAVSLLNATAIAVAEGTDRIIEVVQRTFRAIIHIPRRIRQGLERLLL

SEQ ID NO: 17 - DNA, 2598 bp, synthetic construct
  misc_feature 1..2598: note = pGX1038 - Env Clade B tier 2 TRO.11 DNA Sequence
  source 1..2598: mol_type = other DNA
atgagggcaaaagggattaggaagaactgtcagcacctgtggatttggggaacaatgctgctgggaatgctgatgatctactctgcagccgagcaggggcagctgtgggtgactgtctactatggagtgcctgtctggaaggacgcctctaccacactgttttgcgctagtgacgctaaagcatacgataccgaagtgcacaatgtctgggcaacccatgcctgcgtgccaacagatccaaatccccaggaggtggtcctgggcaacgtgacagaaaacttcaatatgtggaagaacaacatggtggaccagatgcacgaggatatcatttcactgtgggaccagagcctgaagccatgcgtgaaactgacccccctgtgcgtcaccctgaattgtacagataacatcaccaacacaaatactaacagctccaagaactctagtacacatagctataacaattccctggaaggagagatgaaaaattgtagctttaacatcactgcaggcattcgggacaaggtgaagaaagagtacgccctgttctataaactggatgtggtccctatcgaggaagacaaggataccaacaagactacctacagactgaggtcttgcaacactagtgtgattacccaggcctgtcccaaggtcacatttgagcctatcccaattcactattgcgcccctgctggcttcgctatcctgaaatgtaatgacaagaagttcaacggaacaggcccatgcactaacgtgtccaccgtccagtgtacacatgggatcaggcccgtggtctcaacacagctgctgctgaatggaagcctggccgaggaagaggtggtcattcgctctgagaactttacaaacaacgctaagactatcatcgtgcagctgaatgaatccatcgcaattaactgcactcgccctaacaataacacccggagatctatccacattgggccaggacgagctttctacgcaaccggggacatcattggagatatccgacaggcccattgtaatattagtcggaccgagtggaactcaacactgcggcagatcgtgacaaagctgagagaacagctgggcgacccaaacaagactatcattttcaaccagtcaagcggcggggatacagagatcactatgcacagttttaattgcggaggcgaattcttttactgtaacacaactaagctgttcaattcaacctggaacggcaataacaccacagagagcgattccactggggaaaatatcaccctgccctgcaggattaagcagatcattaacctgtggcaggaagtgggaaaggccatgtatgctccccctatcaaaggccagattagctgttcctctaacatcacaggactgctgctgactcgcgacggaggaaataacaatagttcagggcctgaaacattcagaccaggcgggggaaatatgaaggataactggaggagcgagctgtacaagtacaaagtgatcaaaatcgaacccctgggcgtggctcctaccagggcaaagaggcgcgtggtccagcgagagaaacgggctgtgggcactctgggggcaatgttcctgggatttctgggagcagctgggagcaccatgggagcagcatccgtgaccctgacagtccaggccaggctgctgctgtccgggatcgtgcagcagcagaacaatctgctgcgcgcaattgaggcccagcagcacatgctgcagctgaccgtgtggggcatcaagcagctgcaggcccgggtgctggctgtcgaaagatacctgagggaccagcagctgctgggaatctggggctgcagcgggaagctgatttgtactaccaatgtgccctggaacgcttcttggagtaacaagtccctgaacaatatctgggagaacatgacctggatggaatgggagagagaaatcgacaactacacagatctgatctatattctgctggagaagtctcagatccagcaggagaagaacgagcaggaactgctggaactggactcatgggccagcctgtggaactggttcgatattagtaagtggctgtggtacatcaaaatcttcatcatgattgtgggaggactggtcggactgcgaatcgtgtttgcagtcctgagcattgccaaccgcgtgcgacagggctattcccccctgtctttccagactagactgccaacccctcgcggcccagaccgaccagaggggatcgagaaggaaggaggaggacgagacagagatggcagccggcctctggtgcacggactgctggccctgatctgggacgatctgagatccctgtgcctgttctcttatcataggctgcgcgatctgctgctgattgtgactagaaccgtcgagctgctgggacgacggggatgggaactgctgaagtactggtggaacctgctgcagtattggtctcaggagctgaaaaatagtgcagtgtcactgctgaacacaactgcaatcgccgtggctgagggcaccgacagggtcattgaagtggtccagcgcgcctttagagccattctgcatattcccgcccgcattagacagggactggagagagcactgctgtgataa

SEQ ID NO: 18 - protein, 864 aa, synthetic construct
  REGION 1..864: note = pGX1038 - Env Clade B tier 2 TRO.11 Amino Acid Sequence
MRAKGIRKNCQHLWIWGTMLLGMLMIYSAAEQGQLWVTVYYGVPVWKDASTTLFCASDAKAYDTEVHNVWATHACVPTDPNPQEVVLGNVTENFNMWKNNMVDQMHEDIISLWDQSLKPCVKLTPLCVTLNCTDNITNTNTNSSKNSSTHSYNNSLEGEMKNCSFNITAGIRDKVKKEYALFYKLDVVPIEEDKDTNKTTYRLRSCNTSVITQACPKVTFEPIPIHYCAPAGFAILKCNDKKFNGTGPCTNVSTVQCTHGIRPVVSTQLLLNGSLAEEEVVIRSENFTNNAKTIIVQLNESIAINCTRPNNNTRRSIHIGPGRAFYATGDIIGDIRQAHCNISRTEWNSTLRQIVTKLREQLGDPNKTIIFNQSSGGDTEITMHSFNCGGEFFYCNTTKLFNSTWNGNNTTESDSTGENITLPCRIKQIINLWQEVGKAMYAPPIKGQISCSSNITGLLLTRDGGNNNSSGPETFRPGGGNMKDNWRSELYKYKVIKIEPLGVAPTRAKRRVVQREKRAVGTLGAMFLGFLGAAGSTMGAASVTLTVQARLLLSGIVQQQNNLLRAIEAQQHMLQLTVWGIKQLQARVLAVERYLRDQQLLGIWGCSGKLICTTNVPWNASWSNKSLNNIWENMTWMEWEREIDNYTDLIYILLEKSQIQQEKNEQELLELDSWASLWNWFDISKWLWYIKIFIMIVGGLVGLRIVFAVLSIANRVRQGYSPLSFQTRLPTPRGPDRPEGIEKEGGGRDRDGSRPLVHGLLALIWDDLRSLCLFSYHRLRDLLLIVTRTVELLGRRGWELLKYWWNLLQYWSQELKNSAVSLLNTTAIAVAEGTDRVIEVVQRAFRAILHIPARIRQGLERALL

SEQ ID NO: 19 - DNA, 2571 bp, synthetic construct
  misc_feature 1..2571: note = pGX1031 - Env Clade B tier 2 REJO4541.67 DNA Sequence
  source 1..2571: mol_type = other DNA
atgaaagtgaaggggattaggagaaactatcagcacctgtggagatgggggattatgctgctggggattctgatgatttgttccgcaactgaaaagctgtgggtgaccgtctactatggcgtgccagtctggaaagaggccaccacaactctgttctgcgctagcgacgctaaggcatacgatcaggaaatccacaacatttgggccacacatgcttgcgtgcccactgacccaaacccccaggaggtggaactgaagaatgtcaccgagaacttcaacatgtggaaaagcaatatggtggaacagatgcacgaggacatcattagtctgtgggatcagtcactgaagccatgcgtgaaactgacacccctgtgcgtcaccctgaagtgtacagacctgaacgtgactaatagcaactccactgatcattcaaccaatagctccctggaagctaagggcgagatcaagaactgcagcttcaatatcaccacaactccccgggacaagattcagaaagagtacgccatcttttataagcaggacgtggtccctatcaaaaacgataacatcagctacagactgatctcctgcaacacatctgtgatcactcaggcctgtccaaaggtcaccttcgagcctattccaatccactattgcgcccccgctggcttcgctatcctgaagtgtaacgataaagggtttaatgggaccggaccttgcacaaacgtgtccactgtccagtgtacccatggaatcaggccagcaattagcactcagctgctgctgaatggctccctggccgaggacaaggtggtcattcgctctgagaacttcacagataatgccaagatcattatcgtgcacctgaacgaaaccgtcaaaatcaattgcacacgccccaacaacaacactcgaaagagtatccatatcgctcctggcagagccttctacgccactggcgagattatcggggacattaggaaggcatattgtaccatcaacgagagcgaatggaataacaccctgcagaagattgtggtcacactgagggaacagttccgcaacaaaaccatcgtgtttaatcagtctagtggcggcgaccccgaagtgacaatgcacactttcaattgcggaggcgagttcttttactgtaacacagcccagctgtttaattcaagctgggacaccaatacaaacggaaatgatacacagggcccttccgagaataacactattatcctgccatgcaggattaagcagattatcaacatgtggcagcgcgtgggaaaagctatctatgcaccccctatctccggccagattcgatgtctgtctaacatcacagggctgattctgactcgggacgggggaaattcctctctgagttcacctgagatctttaggccaggcgggggagacatgcgagataattggcggtctgaactgtacaagtataaagtggtccagattgagccactgggaatcgcacctacccgcgccaagcggagagctgtgcagagagagaaaagggctgtcggcatcggggcactgttcctgggctttctgggggccgctggatctacaatgggcgcagccagtgtgactctgaccgtccaggcacgacagctgctgagtgggattgtgcagcagcagtcaaacctgctgcgagccatcgaagctcagcagcacctgctgcagctgaccgtgtgggggatcaagcagctgcaggcaagggtgctggccatggagtcttacctgaaagaccagcagctgctgggcatttgggggtgcagtggaaagctgatctgtaccacaactgtgccttggaacacctcttggagtaacaagagcctggatcagatttggaataacatgacatggcgcgagtgggaaaaggagatcgacaactacaccgatctgatctatacactgattgaaaagtcccagaaccagcaggagaaaaatgaacaggagctgctggagctggacaaatgggcctctctgtggaactggttcgatattaccaattggctgtggtacattaagatctttattatggtggtcggcgggctggtgggcctgagaatcgtgttcgcagtcctgtccattatcaaccgagtgcggcaggggtattcacctctgagctttcagacccacctgccagcacctagaggaccagacaggcccgaaggaatcggagaggaaggaggagagcgagactccgatcgctctgggcgaagtgtggacggattcctgccactgatctgggtggatctgcggagcctgttcctgttttcctatcatagactgactgatctgctgctgatcgtgaccagaattgtcgaactgctgggcaggcgcggatggggaatcctgaaatactggtggtcactgctgcagtattggagccaggagctgaagaactcagccgtgagcctgctgaatgcaaccgccattgctgtggcagaacggacagatagaattatcgagatcgtgcagagggtcttccgcgcactgctgcatattccaagacgcattcgacagggatttgagagagcactgctgtgataa

SEQ ID NO: 20 - protein, 855 aa, synthetic construct
  REGION 1..855: note = pGX1031 - Env Clade B tier 2 REJO4541.67 Amino Acid Sequence
MKVKGIRRNYQHLWRWGIMLLGILMICSATEKLWVTVYYGVPVWKEATTTLFCASDAKAYDQEIHNIWATHACVPTDPNPQEVELKNVTENFNMWKSNMVEQMHEDIISLWDQSLKPCVKLTPLCVTLKCTDLNVTNSNSTDHSTNSSLEAKGEIKNCSFNITTTPRDKIQKEYAIFYKQDVVPIKNDNISYRLISCNTSVITQACPKVTFEPIPIHYCAPAGFAILKCNDKGFNGTGPCTNVSTVQCTHGIRPAISTQLLLNGSLAEDKVVIRSENFTDNAKIIIVHLNETVKINCTRPNNNTRKSIHIAPGRAFYATGEIIGDIRKAYCTINESEWNNTLQKIVVTLREQFRNKTIVFNQSSGGDPEVTMHTFNCGGEFFYCNTAQLFNSSWDTNTNGNDTQGPSENNTIILPCRIKQIINMWQRVGKAIYAPPISGQIRCLSNITGLILTRDGGNSSLSSPEIFRPGGGDMRDNWRSELYKYKVVQIEPLGIAPTRAKRRAVQREKRAVGIGALFLGFLGAAGSTMGAASVTLTVQARQLLSGIVQQQSNLLRAIEAQQHLLQLTVWGIKQLQARVLAMESYLKDQQLLGIWGCSGKLICTTTVPWNTSWSNKSLDQIWNNMTWREWEKEIDNYTDLIYTLIEKSQNQQEKNEQELLELDKWASLWNWFDITNWLWYIKIFIMVVGGLVGLRIVFAVLSIINRVRQGYSPLSFQTHLPAPRGPDRPEGIGEEGGERDSDRSGRSVDGFLPLIWVDLRSLFLFSYHRLTDLLLIVTRIVELLGRRGWGILKYWWSLLQYWSQELKNSAVSLLNATAIAVAERTDRIIEIVQRVFRALLHIPRRIRQGFERALL

SEQ ID NO: 21 - DNA, 2568 bp, synthetic construct
  misc_feature 1..2568: note = pGX1032 - Env Clade B tier 2 RHPA4259.7 DNA Sequence
  source 1..2568: mol_type = other DNA
atgagagtgatggggattaggaaaaactaccagcacctgtggaaatgggggactatgctgctgtggctgctgatgatctgttctgcagccgatcagctgtgggtgaccgtctactatggcgtgccagtctggaaggaagcaaacaccacactgttctgcgccagcgacgctaaagcatacgatacagaggcccacaatgtctgggcaacacatgcctgcgtgcccactgacccaaacccccaggaggtggtcctggaaaatgtgacagagaacttcaacatgtggaagaaccacatggtggaacagatgcatgaggacatcatttctctgtgggatcagagtctgaagccctgcgtcaaactgactcctctgtgcgtgaccctgaactgtacagacctggtcaattctaacattacccgcgtggataacactaccgagaaggaaatgaagaactgttcattcaacgtcaccagcggcatccgggacaaggtgcagaaagagtacgccctgctgtataaactggatatcgtgcagattgacaatgataacacctcccacagggacaacacatcttaccgcctgatctcttgcaatactagtgtgattacccaggcctgtcctaagatcagcttcgagcctatcccaattcatttctgcgccccagctggctttgctatcctgaaatgtaatgacaagaagttcaacggaacaggcccctgcactaacgtcagtaccgtgcagtgtacacacgggattagacctgtggtctctacacagctgctgctgaacggaagtctggccgaggaagaggtggtcatcaggagcgaaaatttcactaacaatgtcaagaacatcattgtgcagctgaacgagtcagtgcagatcaattgcactcgacacaacaataacacccggaagagcatcaatattgggcccggaagagctttttatgcaaccgggaaaatcattggagatattcggcaggcccattgtaacatctctagagaaaagtggcagaataccctgaaacagatcgtgaagaaactgagggagcagttcaagaacaaaacaattgcatttgccccaagctccggaggggaccccgaaatcgtgatgcatagcttcaattgcaacggggagttcttttactgtaacacaactaagctgtttacatcaacttggaatagcacttggaactccacctggaataacacagaaggatcaaacagcacagtgatcactctgccttgccgaattcggcagatcattaatatgtggcaggaagtggggaaggccatgtatgctccccctatccagggacagatcaagtgttctagtaacattactggactgctgctgacccgagacggaggagtggataccacaaaggagacattcaggccagggggaggcaatatgaaagataactggaggtccgaactgtacaagtataaagtggtccgcatcgagcctctgggagtggctccaactaaggcaaaacggagagtggtccagcgcgagaagcgagcagtgggcattggggccatgttcctgggatttctgggagcagctgggagtaccatgggagcagcctcaatcaccctgacagtccaggccagactgctgctgagcgggattgtgcagcagcagtccaacctgctgagggcaatcgaagcccagcagcacctgctgcagctgaccgtgtggggcatcaagcagctgcaggccagagtcctggctgtggagaggtacctgaaggatcagcagctgctgggaatttggggctgctccgggaaactgatctgtactaccgctgtgccctggaatgcatcctggtctaacaaatctcaggacacaatctgggggaatatgacttggatgcagtgggagagagaaattgacaactacacagatctgatctataatctgctggaagagagccagaatcagcaggagaagaacgagcaggaactgctggccctggacaaatgggctagtctgtggtcatggttcagcattacccactggctgtggtacatcaagatgtttatcatgattgtcgggggactggtgggactgcgcattgtctttgccgtgctgtccatcgtcaacagagtgaggcagggctattcccctctgtctttccagacccgatttccagctcctcggggaccagatagacccgaaggcattgaagaggaaggaggagagcgagaccgagatcggagtggccgctcagccgacgggttcctggtgctggtctgggtggatctgcggaacctgtgcctgtttagctatcatagactgagggacctgctgctgatcgtcactcgaaccgtggaactgctgggaaggcgcggatgggaggctctgaagtactggtggaatctgctgcagtattggtcccaggagctgaagaaaagcgcagtgtccctgctggacgctatcgcaattgccgtggctgaaggcaccgatcgcatcattgagctgctgcagcgaatcttccgagcctttctgcatattcccacacgcattcgccagggactggagagagcactgcagtgataa

SEQ ID NO: 22 - protein, 854 aa, synthetic construct
  REGION 1..854: note = pGX1032 - Env Clade B tier 2 RHPA4259.7 Amino Acid Sequence
MRVMGIRKNYQHLWKWGTMLLWLLMICSAADQLWVTVYYGVPVWKEANTTLFCASDAKAYDTEAHNVWATHACVPTDPNPQEVVLENVTENFNMWKNHMVEQMHEDIISLWDQSLKPCVKLTPLCVTLNCTDLVNSNITRVDNTTEKEMKNCSFNVTSGIRDKVQKEYALLYKLDIVQIDNDNTSHRDNTSYRLISCNTSVITQACPKISFEPIPIHFCAPAGFAILKCNDKKFNGTGPCTNVSTVQCTHGIRPVVSTQLLLNGSLAEEEVVIRSENFTNNVKNIIVQLNESVQINCTRHNNNTRKSINIGPGRAFYATGKIIGDIRQAHCNISREKWQNTLKQIVKKLREQFKNKTIAFAPSSGGDPEIVMHSFNCNGEFFYCNTTKLFTSTWNSTWNSTWNNTEGSNSTVITLPCRIRQIINMWQEVGKAMYAPPIQGQIKCSSNITGLLLTRDGGVDTTKETFRPGGGNMKDNWRSELYKYKVVRIEPLGVAPTKAKRRVVQREKRAVGIGAMFLGFLGAAGSTMGAASITLTVQARLLLSGIVQQQSNLLRAIEAQQHLLQLTVWGIKQLQARVLAVERYLKDQQLLGIWGCSGKLICTTAVPWNASWSNKSQDTIWGNMTWMQWEREIDNYTDLIYNLLEESQNQQEKNEQELLALDKWASLWSWFSITHWLWYIKMFIMIVGGLVGLRIVFAVLSIVNRVRQGYSPLSFQTRFPAPRGPDRPEGIEEEGGERDRDRSGRSADGFLVLVWVDLRNLCLFSYHRLRDLLLIVTRTVELLGRRGWEALKYWWNLLQYWSQELKKSAVSLLDAIAIAVAEGTDRIIELLQRIFRAFLHIPTRIRQGLERALQ

SEQ ID NO: 23 - DNA, 2568 bp, synthetic construct
  misc_feature 1..2568: note = pGX1040 - Env Clade C tier 2 Du123.6 DNA Sequence
  source 1..2568: mol_type = other DNA
atgagagtcaagggcattcagcgcaactggcctcagtggtggatttggggcattctgggattctggatgattattatctgtagagtcgtgggcaacctgtgggtgacagtctactatggggtgccagtctggactgaggcaaagaccacactgttctgcgccagcgacgcaaaagcctacgagagagaagtgcacaatgtctgggcaactcatgcctgtgtgcccaccgatccaaatccccaggaaatcgtgctgggcaacgtcaccgagaattttaacatgtggaagaacgacatggtggatcagatgcacgaagacatcatttctatctgggatcagagtctgaagccttgcgtgaaactgactccactgtgcgtcactctgaattgtaccgacgtgaaggtcaatgccaccagcaacgggactaccacatacaacaattccattgattctatgaacggagaaatcaagaactgtagcttcaacatcactaccgagatccgcgacaagaaacagaaagtgtacgccctgttttatcgaccagatgtggtccccctgaatgagaacagctcctcttatattctgatcaattgcaacacatccacaactacccaggcttgtcctaaggtgtctttcgaccctattccaatccactactgcgctccagcaggctatgccatcctgaagtgtaacaacaagaccttcaacgggactggaccctgccacaacgtgtccaccgtccagtgtacacatggcatcaagcctgtggtctctacccagctgctgctgaatgggagtctggccgaggaagagatcattatccggtctgagaatctgaccaacaatgctaagacaattatcgtgcatctgaacgagagcattgaaatcgtctgcacaagaccaaacaataacactcgaaaatccattcggatcggccccgggcagactgtgtacgctaccaacgacattatcggggatattcggcaggcacactgtaatatcagcaagacaaaatggaacacaactctggagaaggtgaaagaaaagctgaaagagcatttcccctcaaaggccatcacttttcagcctcacagcggcggggacctggaagtgaccacacattctttcaattgcagaggcgagttcttttactgtgatactaccaagctgtttaatgagagtaatctgaacacaactaacaccacaactctgaccctgccctgccggatcaagcagatcgtgaacatgtggcagggagtcggccgcgctatgtatgcaccccctgtggagggaaatattacctgtaacagttcaatcacaggcctgctgctggtgagggacggaggcaatacatcaaacagcactcccgaaattttcagacctgggggaggcaatatgaaggataactggaggtccgaactgtacaagtataaagtggtcgagatcaaaccactgggcgtggcacccacaaaggccaaacggagagtggtcgagcgggaaaagagagccgtggggattggagctgtcctgttcggctttctgggagcagctggcagcaccatgggagcagcctctatcactctgaccgtgcaggcacgacagctgctgagcggcattgtccagcagcagtccaacctgctgagagccatcgaggctcagcagcacatgctgcagctgaccgtgtggggcattaagcagctgcaggcccgggtgctggcaatcgaacggtacctgaaggaccagcagctgctgggactgtggggatgctctggaaaactgatttgtcctaccacagtgccatggaatagctcctggagtaacaagtcacagactgacatctgggataatatgacctggatgcagtgggaccgcgagattagtaactacacaggcactatctataaactgctggaagagtcacagaatcagcaggagaagaacgaaaaagacctgctggccctggatagttggaagaatctgtggtcatggttcgatatcaccaactggctgtggtacatcaagatctttattatgatcgtggggggactgattgggctgaggattatcttcggagtgctgagcatcgtgaagcgagtccggcagggatatagccctctgtcctttcagaccctgacacccaatcctcgcggactggacaggctgggccgcattgaagaggaaggcggggagcaggacaaagatcgaagcatccgactggtgaacggcttcctggcactggcttgggacgatctgaggtcactgtgcctgttcagctatcatagactgagggattttatcctggtggctgcacgcgcagtcgaactgctggggagatctagtctgaggggactgcagcgaggatgggaggccctgaagtacctgggaaatctggtgcagtatggaggcctggaactgaaaaggcgcgctatctccctgttcgacaccattgcaatcgccgtggctgaaggcacagatagaattctggaggtcatcctgagaattatcagggccattcgcaacatccccacccgcatccgacaggggtttgaggccgctctgctgtgataa

SEQ ID NO: 24 - protein, 854 aa, synthetic construct
  REGION 1..854: note = pGX1040 - Env Clade C tier 2 Du123.6 Amino Acid Sequence
MRVKGIQRNWPQWWIWGILGFWMIIICRVVGNLWVTVYYGVPVWTEAKTTLFCASDAKAYEREVHNVWATHACVPTDPNPQEIVLGNVTENFNMWKNDMVDQMHEDIISIWDQSLKPCVKLTPLCVTLNCTDVKVNATSNGTTTYNNSIDSMNGEIKNCSFNITTEIRDKKQKVYALFYRPDVVPLNENSSSYILINCNTSTTTQACPKVSFDPIPIHYCAPAGYAILKCNNKTFNGTGPCHNVSTVQCTHGIKPVVSTQLLLNGSLAEEEIIIRSENLTNNAKTIIVHLNESIEIVCTRPNNNTRKSIRIGPGQTVYATNDIIGDIRQAHCNISKTKWNTTLEKVKEKLKEHFPSKAITFQPHSGGDLEVTTHSFNCRGEFFYCDTTKLFNESNLNTTNTTTLTLPCRIKQIVNMWQGVGRAMYAPPVEGNITCNSSITGLLLVRDGGNTSNSTPEIFRPGGGNMKDNWRSELYKYKVVEIKPLGVAPTKAKRRVVEREKRAVGIGAVLFGFLGAAGSTMGAASITLTVQARQLLSGIVQQQSNLLRAIEAQQHMLQLTVWGIKQLQARVLAIERYLKDQQLLGLWGCSGKLICPTTVPWNSSWSNKSQTDIWDNMTWMQWDREISNYTGTIYKLLEESQNQQEKNEKDLLALDSWKNLWSWFDITNWLWYIKIFIMIVGGLIGLRIIFGVLSIVKRVRQGYSPLSFQTLTPNPRGLDRLGRIEEEGGEQDKDRSIRLVNGFLALAWDDLRSLCLFSYHRLRDFILVAARAVELLGRSSLRGLQRGWEALKYLGNLVQYGGLELKRRAISLFDTIAIAVAEGTDRILEVILRIIRAIRNIPTRIRQGFEAALL

SEQ ID NO: 25 - DNA, 2532 bp, synthetic construct
  misc_feature 1..2532: note = pGX1021 - Env Clade C tier 2 ZM53M.PB12 DNA Sequence
  source 1..2532: mol_type = other DNA
atgcgagtccgggagattcctcgaaactatcagcagtggtggatttgggggattctgggcttctggatgctgatgatttgtagcgtggtggggaatctgtgggtgaccgtctactatggagtgcccgtctggagggaggctaagaccacactgttctgcgcaagcgacgctaaagcatacgaacgcgaggtgcacaatgtctgggcaactcatgcctgcgtgcctaccgatccaaatccccaggaaatggtgctggagaacgtcacagaaaactttaatatgtggaagaacgacatggtggatcagatgcaggaggacatcatttcactgtgggatcagagcctgaaaccatgcgtgaagctgactcccctgtgcgtcaccctgaactgtagtaagctgaacaatgcaaccgacggagagatgaaaaattgttcattcaacgccactaccgaactgagggataagaaaaagcaggtgtacgccctgttttataagctggacatcgtccctctggatggccggaacaatagctccgagtatagactgattaactgcaatacctctacaatcactcaggcatgtccaaaagtgagtttcgaccctattccaatccactactgcgcccccgctggctatgccatcctgaaatgtaacaataagacttttaatgggaccggaccttgccacaacgtgtctacagtccagtgtactcatggcattaagccagtgatcagcactcagctgctgctgaacgggtccaccgctgaggaagacatcattatcaggagtgagaatctgacaaacaatgcaaagactattatcgtgcatctgaacgaaagcattgaaatcgagtgcacacgccccggcaacaatactaggaaatccattcgcatcggccctgggcaggctttctttgcaacaactaatattatcggggatatccggcaggcctactgtattatcaacaaggctaattggaccaacacactgcacagagtgtcaaaaaagctggaggaacatttcccaaacaaaacaattaactttaattctagttcaggcggggacctggagatcaccacacacagcttcaattgcggaggcgaattcttttactgtaacaccagctccctgtttaatggcacctacaacgacacagatatctacaattccacagatattatcctgctgtgcagaatcaagcagattatcaacatgtggcaggaagtgggcagggccatgtatgctccccctattgaagggaatatcacctgttctagtaacatcaccggactgctgctgacacgcgacgggggactgaccaatgaatctaaggagacattccgacccggcgggggagacatgcgagataactggcggagtgagctgtacaaatataaggtggtcgaaattaagcccctgggcatcgctcctactaaagcaaagcggagagtggtcgaacgcgagaaacgagcagtgggactgggcgccatgttcctggggtttctgggagccgctggcagtaccatgggagcagcctcaatcactctgaccgtgcaggcacgacagctgctgagcggcattgtccagcagcagaacaatctgctgagagcaatcgaggcccagcagcatatgctgcagctgaccgtgtggggcattaagcagctgcaggcccgcgtcctggctatcgagcgatacctgaaggaccagcagctgctgggactgtggggatgctccggcaaactggtgtgcactaccgccgtcccctggaattcaagctggagtaacaagtcacaggaggacatttggaacaatacaacttggatgcagtgggataaagaagtgtccaactacacaaaaactatctataagctgctggagaaatctcagaatcagcaggaggaaaacgaaaaggacctgctggccctggattcatggaacaatctgtggaattggttcgatatcagcaactggctgtggtacatcaagatctttattatgatcgtgggcgggctgattgggctgcggattatcttcgccgtgctgagcatcgtgaatagggtccgccagggatatagccctctgtcctttcagaccctgacacagaacccaagaggcctggaccggctggggagaatcgaggaagagggaggcgagcaggaccgagatcggtccgtgaggctggtcaacgggttcctggctctgttttgggacgatctgcgctccctgtgcctgttctcttaccacagactgagggacttcatcctgatcgcaaccagggtggtcgagctgctgggccgctcctctctgaaggggctgcagagaggatgggaagccctgagatacctgggatctagggtgcagtattggggcctggagctgaaaaagtctgctattagtctgttcgacacaattgcaatcgccgtggctgagggcactgatcgaattatcgaactgatccagcggtcctggagagctattcggaacatcccaagaagaatccgccagggctttgagaccgcactgctgtgataa

SEQ ID NO: 26 - protein, 842 aa, synthetic construct
  REGION 1..842: note = pGX1021 - Env Clade C tier 2 ZM53M.PB12 Amino Acid Sequence
MRVREIPRNYQQWWIWGILGFWMLMICSVVGNLWVTVYYGVPVWREAKTTLFCASDAKAYEREVHNVWATHACVPTDPNPQEMVLENVTENFNMWKNDMVDQMQEDIISLWDQSLKPCVKLTPLCVTLNCSKLNNATDGEMKNCSFNATTELRDKKKQVYALFYKLDIVPLDGRNNSSEYRLINCNTSTITQACPKVSFDPIPIHYCAPAGYAILKCNNKTFNGTGPCHNVSTVQCTHGIKPVISTQLLLNGSTAEEDIIIRSENLTNNAKTIIVHLNESIEIECTRPGNNTRKSIRIGPGQAFFATTNIIGDIRQAYCIINKANWTNTLHRVSKKLEEHFPNKTINFNSSSGGDLEITTHSFNCGGEFFYCNTSSLFNGTYNDTDIYNSTDIILLCRIKQIINMWQEVGRAMYAPPIEGNITCSSNITGLLLTRDGGLTNESKETFRPGGGDMRDNWRSELYKYKVVEIKPLGIAPTKAKRRVVEREKRAVGLGAMFLGFLGAAGSTMGAASITLTVQARQLLSGIVQQQNNLLRAIEAQQHMLQLTVWGIKQLQARVLAIERYLKDQQLLGLWGCSGKLVCTTAVPWNSSWSNKSQEDIWNNTTWMQWDKEVSNYTKTIYKLLEKSQNQQEENEKDLLALDSWNNLWNWFDISNWLWYIKIFIMIVGGLIGLRIIFAVLSIVNRVRQGYSPLSFQTLTQNPRGLDRLGRIEEEGGEQDRDRSVRLVNGFLALFWDDLRSLCLFSYHRLRDFILIATRVVELLGRSSLKGLQRGWEALRYLGSRVQYWGLELKKSAISLFDTIAIAVAEGTDRIIELIQRSWRAIRNIPRRIRQGFETALL

SEQ ID NO: 27 - DNA, 2580 bp, synthetic construct
  misc_feature 1..2580: note = pGX1020 - Env Clade C tier 2 Du422.1 DNA Sequence
  source 1..2580: mol_type = other DNA
atgcgagtccgggggattcctcgaaactggcctcagtggtggatctgggggattctgggattctggatgattatcatctgtagggtcgtgggaaacctggatctgtgggtgacagtctactatggcgtgcctgtctggaaagaagctaagaccacactgttctgcgcaagcgacgcaaaagcctacgataaggaggtgcacaatgtctgggcaacacatgcctgcgtgccaactgacccaaatccccaggaaatcgtgctggagaacgtcaccgaaaacttcaacatgtggaagaacgacatggtggatcagatgcacgaggacatcatttcactgtgggatcagagcctgaaaccctgcgtgaagctgacacctctgtgcgtcactctgaactgtaaaaatgtgaacatctccgctaatgcaaacgccaccgctacactgaatagctccatgaacggcgagattaagaattgttctttcaacactaccacagaactgagagacaagaaacagaaagtgtacgccctgttttataagccagatgtggtccccctgaatggcggggagcacaacgaaacaggggagtatatcctgattaattgcaactctagtactatcacccaggcatgtcccaaggtgtccttcgatcctatcccaattcattactgcgcacctgccggatatgccattctgaaatgtaacaataagacttttaatgggaccggaccatgcaacaatgtgagcacagtccagtgtactcacggcatcaagcccgtggtctccacccagctgctgctgaacgggtctctggccgaggaagagatcattgtgagatccgaaaatctgaccaacaacatcaaaacaatcattgtgcatctgaacaaaagcgtcgagattaagtgcaccaggccaaacaataacacacgaaagtccgtgcgaatcggaccaggacagaccttctacgcaacaggggagatcattggagacatcagggaagctcactgtaatattagccgcgagacttggaactccaccctgatccaggtgaaggagaaactgcgcgaacactataacaagaccattaagttcgagccctcaagcggaggcgacctggaagtgactacccatagttttaactgccggggcgagttcttttactgtgatacaactaagctgttcaatgaaaccaagctgtttaacgagagcgaatatgtggacaacaagacaatcattctgccttgcagaatcaagcagatcattaacatgtggcaggaagtgggaagggctatgtacgcaccccctatcgaaggcaacatcacttgtaagtctaacatcactgggctgctgctgacctgggatgggggagagaacagtaccgaaggcgtgttcagacccggcgggggaaatatgaaagacaactggaggtcagagctgtacaagtataaagtggtcgaaatcaagcctctgggggtggccccaaccaagagcaaaaggaaggtggtcggaagggagaagcgagcagtgggactgggagccgtcctgctggggtttctgggagccgctggctctacaatgggagcagccagtatcacactgactgtccaggctcgccagctgctgtcaggcatcgtgcagcagcagagcaatctgctgcgggccattgaggctcagcagcacctgctgcagctgactgtctggggcatcaaacagctgcagacccgcgtgctggccattgagcgatacctgaaagatcagcagctgctggggctgtggggatgctctggcaagctgatctgtgctacagcagtgccctggaattcctcttggagcaacaagtccctgggcgacatttgggataacatgacttggatgcagtgggaccgcgagatcagtaattataccaacacaattttccgactgctggaagattcacagaatcagcaggagaagaacgagaaggacctgctggctctggatagctggaaaaatctgtggaactggttcgacatcactaattggctgtggtacatcaagatcttcatcatgattgtcggcgggctgatcgggctgagaatcattttcggagtgctggccattgtgaaacgggtcagacagggctattctcctctgagttttcagaccctgatccccaaccctaggggaccagatcgactgggccggattgaagaggaaggaggcgagcaggacaaggatagatccatcaggctggtgtctggcttcctggccctggcttgggacgatctgcgcagtctgtgcctgttctcataccatcagctgcgagactttatcctgaccgctgcacgggccgctgagctgctggggcggagttcactgagaggcctgcagagggggtgggaagtcctgaaatacctgggcaatctggtgcagtattgggggctggagctgaagcggtctgccatcaacctgtttgacacaatcgcaattgccgtcgctgagggcactgatcggatcattgaagtgatccagagaatttgccgagctattcgctacattcctacccgcattcgccagggatttgaagccgctctgctgtgataa

SEQ ID NO: 28 - protein, 858 aa, synthetic construct
  REGION 1..858: note = pGX1020 - Env Clade C tier 2 Du422.1 Amino Acid Sequence
MRVRGIPRNWPQWWIWGILGFWMIIICRVVGNLDLWVTVYYGVPVWKEAKTTLFCASDAKAYDKEVHNVWATHACVPTDPNPQEIVLENVTENFNMWKNDMVDQMHEDIISLWDQSLKPCVKLTPLCVTLNCKNVNISANANATATLNSSMNGEIKNCSFNTTTELRDKKQKVYALFYKPDVVPLNGGEHNETGEYILINCNSSTITQACPKVSFDPIPIHYCAPAGYAILKCNNKTFNGTGPCNNVSTVQCTHGIKPVVSTQLLLNGSLAEEEIIVRSENLTNNIKTIIVHLNKSVEIKCTRPNNNTRKSVRIGPGQTFYATGEIIGDIREAHCNISRETWNSTLIQVKEKLREHYNKTIKFEPSSGGDLEVTTHSFNCRGEFFYCDTTKLFNETKLFNESEYVDNKTIILPCRIKQIINMWQEVGRAMYAPPIEGNITCKSNITGLLLTWDGGENSTEGVFRPGGGNMKDNWRSELYKYKVVEIKPLGVAPTKSKRKVVGREKRAVGLGAVLLGFLGAAGSTMGAASITLTVQARQLLSGIVQQQSNLLRAIEAQQHLLQLTVWGIKQLQTRVLAIERYLKDQQLLGLWGCSGKLICATAVPWNSSWSNKSLGDIWDNMTWMQWDREISNYTNTIFRLLEDSQNQQEKNEKDLLALDSWKNLWNWFDITNWLWYIKIFIMIVGGLIGLRIIFGVLAIVKRVRQGYSPLSFQTLIPNPRGPDRLGRIEEEGGEQDKDRSIRLVSGFLALAWDDLRSLCLFSYHQLRDFILTAARAAELLGRSSLRGLQRGWEVLKYLGNLVQYWGLELKRSAINLFDTIAIAVAEGTDRIIEVIQRICRAIRYIPTRIRQGFEAALL

SEQ ID NO: 29 - DNA, 2601 bp, synthetic construct
  misc_feature 1..2601: note = pGX1019 - Env Clade C tier 2 Cap210.2.00.E8 DNA Sequence
  source 1..2601: mol_type = other DNA
atgagggtcatgggcattcagcgcaactggcagcagtggggcatctggggcattctgggcttctggctgctgatgatttgttcagggatgggaaacctgtgggtgacagtctactatggcgtgcctgtctggaaggaggccaaaaccacactgttttgcgctagcgacgcaaagggctacgatactgaagtgcacaacgtctgggccactcatgcttgcgtgccaaccgaccccaatcctcaggagatcgtgctggaaaacgtcaccgagaacttcaatatgtggaaaaatgacatggtggatcagatgcaccaggacatcatttcactgtgggatcagagcctgaagccctgcgtgaaactgacccctctgtgcgtcacactgaattgttccgacgccacttacaacaatggcaccaactctactgataccatgaagatctgtagtttcaatgctactaccgaactgcgggacaagaaaaagaaagagtacgcactgttttatagactggatatcgtgcctctgaagaacgagtcagaaagccagaatttcagtgagtatatcctgattaactgcaatacatcaactatcgcccaggcttgtcccaaagtgagctttgatccaatccccattcactactgcgcacctgccggctatgctattctgaagtgtaacaacaagaccttcaacggcaccgggccatgcaacaacgtgagcacagtccagtgtactcatgggatcaagcccgtggtctcaacacagctgctgctgaacggaagcctggccgaggaagaggtggtcatccggtctgaaaacatcagtaataatgtgaagaccatcattgtccacctgaacgagagtgtgaatattacatgcatcaggcctggcaacaatactcggagatcaatccgcattggaccaggccaggccttctacgccatgggcgacatcattgggaacatcagagaggcacattgcaatattagcgaaaaggcctggaacgagactctgaagaaagtcgtggagaaactggtgaaatacttccccaacaaaaccatcgaatttgctccccctgtgggcggggatctggagattacaactcacagcttcaattgcggaggcgagttcttttattgtaacaccacaaagctgtttaactccacacataattccaccgactctacagtgaatagtactgattcaaccgccgagacaggcaactctaccaacacaaatatcaccctgccctgccgaattcggcagatcattaatatgtggcaggaagtggggagggctatgtatgcaccaccctccaagggaaacattacctgtatctctaatattacaggactgctgctgactcgcgacgggggagaaaacaaaaccgagaacaatgatacagagatcttccgacctggcgggggagacatgaaggataattggagaagcgaactgtacaagtataaagtggtcgagatcaagcctctgggcgtggcacctacaagagccaagaggcgcgtggtcgagagggaaaaacgcgctgtggggatcggagcagtcttcctgggctttctgggagcagctggaagtaccatgggagcagcctcaattactctgaccgtgcaggcacgacagctgctgagcgggatcgtccagcagcagtccaacctgctgagagccattgaggctcagcagcacatgctgcagctgaccgtgtgggggatcaagcagctgcagacaagagtcctggccattgagaggtacctgaaggaccagcagctgctgggaatctggggatgcagcggaaaactgatttgtactaccaacgtgccatggaatagctcctggagcaataagtcctatggcgacatctgggataacatgacctggatgcagtgggacagggaaatcaacaactacacaaacactatctaccgcctgctggaggattcccagaaccagcaggagaagaatgaacaggacctgctggccctggataaatggcagtctctgtggagttggttctcaatctctagttggctgtggtacatcaagatcttcatcatggtggtcggcgggctgatcggactgaggatcattttcgctgtgctgtccattgtgaacagagtcaggcagggctatagcccactgtccctgcagaccctgcctccaaatccccgagaactggaccggctgggaggcatcgaagaggaagggggagagcaggatcgaggccgatccgtgaggctggtctctgggttcctgccactggcatgggacgatctgcgctctctgtgcctgttttgttaccatcggctgagagacctgctgctgatcacaactcgcgccgtggaactgctggctcgaagtatcctgaagggactgcagcggggctgggagattctgaaatacctggggtccctggtgcagtattggggacaggaactgaagaaatctgccatcaacctgctggacaccacagctattgcagtggccgaagctgcagatagaatcctggagctgctgcagagaatttggagagggatttgtaatgtgcctacccgcatccgacagggctttgaagccgctctgcagtgataa

SEQ ID NO: 30 - protein, 865 aa, synthetic construct
  REGION 1..865: note = pGX1019 - Env Clade C tier 2 Cap210.2.00.E8 Amino Acid Sequence
MRVMGIQRNWQQWGIWGILGFWLLMICSGMGNLWVTVYYGVPVWKEAKTTLFCASDAKGYDTEVHNVWATHACVPTDPNPQEIVLENVTENFNMWKNDMVDQMHQDIISLWDQSLKPCVKLTPLCVTLNCSDATYNNGTNSTDTMKICSFNATTELRDKKKKEYALFYRLDIVPLKNESESQNFSEYILINCNTSTIAQACPKVSFDPIPIHYCAPAGYAILKCNNKTFNGTGPCNNVSTVQCTHGIKPVVSTQLLLNGSLAEEEVVIRSENISNNVKTIIVHLNESVNITCIRPGNNTRRSIRIGPGQAFYAMGDIIGNIREAHCNISEKAWNETLKKVVEKLVKYFPNKTIEFAPPVGGDLEITTHSFNCGGEFFYCNTTKLFNSTHNSTDSTVNSTDSTAETGNSTNTNITLPCRIRQIINMWQEVGRAMYAPPSKGNITCISNITGLLLTRDGGENKTENNDTEIFRPGGGDMKDNWRSELYKYKVVEIKPLGVAPTRAKRRVVEREKRAVGIGAVFLGFLGAAGSTMGAASITLTVQARQLLSGIVQQQSNLLRAIEAQQHMLQLTVWGIKQLQTRVLAIERYLKDQQLLGIWGCSGKLICTTNVPWNSSWSNKSYGDIWDNMTWMQWDREINNYTNTIYRLLEDSQNQQEKNEQDLLALDKWQSLWSWFSISSWLWYIKIFIMVVGGLIGLRIIFAVLSIVNRVRQGYSPLSLQTLPPNPRELDRLGGIEEEGGEQDRGRSVRLVSGFLPLAWDDLRSLCLFCYHRLRDLLLITTRAVELLARSILKGLQRGWEILKYLGSLVQYWGQELKKSAINLLDTTAIAVAEAADRILELLQRIWRGICNVPTRIRQGFEAALQ

SEQ ID NO: 31 - DNA, 2532 bp, synthetic construct
  misc_feature 1..2532: note = pGX1041 - Env Clade C tier 2 Du151.2 DNA Sequence
  source 1..2532: mol_type = other DNA
atgcgcgtgcgggagattctgcgaaactatcagcagtggtggatttgggggactctgggattctggatgctgatgatttgtaatgtggtgggaaacctgtgggtgaccgtctactatggcgtgcccgtctggaaagaggccaagaccacactgttttgcgcttctgacgccaaagcttacgataaggaagtgcacaatgtctgggctacacatgcatgtgtgcctactgaccctaatccacaggagatcgtgctggaaaacgtcacagagaatttcaacatgtggaagaacgacatggtggatcagatgcacgaggacatcatttcactgtgggatcagagcctgaaaccatgcgtgaagctgacccccctgtgcgtcacactgaattgtactaacgcacccgcctacaacaatagtatgcatggcgaaatgaaaaattgtagcttcaacactaccacagagatcagagacaggaaacagaaggcttacgcactgttctataagcctgatgtggtcccactgaatcggagagaggaaaacaatgggaccggagagtatattctgatcaattgcaacagctccacaatcactcaggcctgtccaaaggtgacatttgatcccattcctatccactactgcgcccccgctggctatgctattctgaaatgtaacaataagaccttcaacggcacagggccttgcaacaatgtcagtactgtccagtgtacccatgggatcaatccagtggtctccacccagctgctgctgaacggatctctggccgaggaagagatcattatccggagcgagaatctgaccaacaacatcaaaacaatcatcgtgcacctgaacaagtcagtggaaattgtctgcacccgccctaacaataacacaaggcgcagcattcgaatcggaccaggccagacattctacgcaactggcgaaattatcgggaatatcagggaggcccattgtaacattagcaagtctagttggacctccacactggagcaggtgaagaaaaagctgaaagaacactacaataagacaatcgagttcaacccacctagcggaggggacctggaagtgactacccattcctttaattgcagaggcgagttcttttattgtaacacaactaagctgttcagcaataacagtgattcaaataacgagactatcaccctgccatgcaaaattaagcagattatcaacatgtggcagaaagtggggcgggccatgtatgctccacccatcgagggaaatattacctgtaaatccaacatcactggcctgctgctgaccagagacggaggcaagaataccacaaacgagatttttaggcccgggggaggcaatatgaaagataactggcgctccgaactgtacaaatataaggtggtcgagatcgaaccactgggagtggcacctactaaatctaagcgacgggtggtcgagcgagaaaagcgagctgtgggactgggagcagtcctgctgggcttcctgggagcagctggatctaccatgggagcagccagtatcacactgactgtgcaggccaggcagctgctgtcagggatcgtccagcagcagagcaacctgctgcgcgcaattgaggcccagcagcacatgctgcagctgactgtgtggggcatcaagcagctgcagaccagagtcctggcaattgaaaggtacctgaaagaccagcagctgctgggactgtggggatgcagcggaaagattatctgtactaccgccgtgccttggaattcaagctggagcaacaagtcccaggaggacatctgggataatatgacatggatgcagtgggaccgggaaatctctaactacaccggcacaatctacagactgctggaggatagtcagaatcagcaggagaaaaacgaaaaggacctgctggccctggattcttggaaaaatctgtggaactggttcaatatcaccaactggctgtggtacattaagatctttattatgatcgtggggggactgatcggcctgaggattatctttggggtgctggccattgtgaaacgcgtccgacagggctattctcccctgagtttccagactctgaccccaagccccagaggccctgacagactgggaaggatcgaagaggaaggcggggagcaggataagaatcgctccattcgactggtgtctgggttcctggcactggcctgggacgatctgcggagtctgtgcctgttttcataccaccggctgagagacctgatcctggtggtcaccagagctgtggaactgctgggacgctcctctctgcgaggactgcagcgaggatgggaggcactgaagtacctgggcaacctggtgcagtatggaggcctggaactgaaaaggtccgctatcaagctgtttgacacaattgctatcgcagtggccgaagggactgatcgcatcctggaggtcatccagcggatttgcagagccattaggcatattcccatcaggattcgccagggattcgaggctgcactgctgtgataa

SEQ ID NO: 32 - protein, 842 aa, synthetic construct
  REGION 1..842: note = pGX1041 - Env Clade C tier 2 Du151.2 Amino Acid Sequence
MRVREILRNYQQWWIWGTLGFWMLMICNVVGNLWVTVYYGVPVWKEAKTTLFCASDAKAYDKEVHNVWATHACVPTDPNPQEIVLENVTENFNMWKNDMVDQMHEDIISLWDQSLKPCVKLTPLCVTLNCTNAPAYNNSMHGEMKNCSFNTTTEIRDRKQKAYALFYKPDVVPLNRREENNGTGEYILINCNSSTITQACPKVTFDPIPIHYCAPAGYAILKCNNKTFNGTGPCNNVSTVQCTHGINPVVSTQLLLNGSLAEEEIIIRSENLTNNIKTIIVHLNKSVEIVCTRPNNNTRRSIRIGPGQTFYATGEIIGNIREAHCNISKSSWTSTLEQVKKKLKEHYNKTIEFNPPSGGDLEVTTHSFNCRGEFFYCNTTKLFSNNSDSNNETITLPCKIKQIINMWQKVGRAMYAPPIEGNITCKSNITGLLLTRDGGKNTTNEIFRPGGGNMKDNWRSELYKYKVVEIEPLGVAPTKSKRRVVEREKRAVGLGAVLLGFLGAAGSTMGAASITLTVQARQLLSGIVQQQSNLLRAIEAQQHMLQLTVWGIKQLQTRVLAIERYLKDQQLLGLWGCSGKIICTTAVPWNSSWSNKSQEDIWDNMTWMQWDREISNYTGTIYRLLEDSQNQQEKNEKDLLALDSWKNLWNWFNITNWLWYIKIFIMIVGGLIGLRIIFGVLAIVKRVRQGYSPLSFQTLTPSPRGPDRLGRIEEEGGEQDKNRSIRLVSGFLALAWDDLRSLCLFSYHRLRDLILVVTRAVELLGRSSLRGLQRGWEALKYLGNLVQYGGLELKRSAIKLFDTIAIAVAEGTDRILEVIQRICRAIRHIPIRIRQGFEAALL

SEQ ID NO: 33 - DNA, 2577 bp, synthetic construct
  misc_feature 1..2577: note = pGX1042 - Env Clade C tier 2 Du156.12 DNA Sequence
  source 1..2577: mol_type = other DNA
atgagagtgcggggcattcctcgcaactggcctcagtggtggacctggggcattctgggattctggatgattattatgtgcaaagtggccgggaacagttgggtgactgtctactatggagtgcccgtctggaccgaagctaagaccacactgttctgcgcatctgacgccaaagcttacgagaaggaagtgcacaatgtctgggcaacccatgcctgtgtgcctacagatcctaatccacaggagatcttcctgaaaaacgtgaccgaaaattttaacatgtggaagaacgacatggtcgatcagatgcacgaggacatcattagcctgtgggatcagtccctgaaaccctgcgtgaagctgacccctctgtgcgtgacactgaattgtgtcacttacaacaatagcatgaacagctccgctacctataacaattctatgaacggcgagatcaaaaattgtagtttcaacactaccacagaactgcgagacaagaaacagaaggtgtacgccctgttttataggacagatgtggtccctctgaacaacaacaacaacaactcagagtacatcctgatcaattgcaacactagcaccattacacaggcttgtcctaaagtgtccttcgaccccattcctatccactactgcgcaccagccggctatgccatcctgaagtgtacagataagaagttcaacggcactgggtcttgcaacaatgtcagtactgtccagtgtacccatgggatcaaaccagtggtcagcacccagctgctgctgaacggcagcctggcagaggaagagatcattatcaaatccgagaatctgaccgacaacattaagacaattatcgtgcagctgaatcagtccattggcatcaactgcactagaccaaacaataacacccggaagtctgtgagaatcggacccggccagacattctatgccactggggacattatcggagatattcgccaggctcactgtaacatctctcgaaatcagtggaacgagaccctggaacaggtgaagaaaaagctgggagagcacttccataaccagacaaaaattaagttcgagcccccttctggcggggatctggaaatcactacccatagtttcaactgcagaggcgaattcttttactgtaataccgcagacctgtttaccaacgccacaaaactggtgaatgataccgagaacaaggccgtcattacaatcccatgccgcatcaagcagattatcaatatgtggcagggggtgggacgggctatgtatgcaccacccattgagggcaacatcacatgtaatagcaacatcactggactgctgctgaccagggacggaggaggaaatgtgacagagattaaccgaactgaaatctttcggcccggaggcgggaatatgaaagataattggagaaacgagctgtacaaatataaggtggtcgaaatcaagcctctgggagtggcaccaactggcgccaaaaggaaggtggtcaaaagagagaagagggcagtgggactgggagctgtcctgttcgggtttctgggagcagctggctccacaatgggagcagcctctatcactctgaccgctcaggcaagacagctgctgagtgggattgtgcagcagcagtcaaacctgctgagggccatcgaagctcagcagcacatgctgcagctgaccgtgtggggcattaagcagctgcaggctagagtcctggcaatcgagaggtacctgaaagaccagcagctgctgggactgtggggatgctccggcaagctgatttgtacaactaatgtgccctggaactctagttggtccaacaagtctcagaccgatatctggaataacaccacatggatgcagtgggagagggaaatttcaaactacacagacactatctatcgcctgctggaggatagccagaatcagcaggaagagaacgaaaaggacctgctggccctggatcgctggcagaatctgtggaactggttcgacatcaccaattggctgtggtacatcaagatctttattatgatcgtgggaggcctgattggcctgcgcattatcttcggggtcctgagcatcgtgaagcgagtccgggaaggctatagtcctctgtcatttcagaccctgacaccaactcccagaggcctggaccgcctgggacgaattgaagaggaagggggagagcaggacaaggatcggagcatcagactggtgaacgggttcctggccctggcttgggacgatctgaggtcactgtgcctgttcagctaccatcagctgcgggattttattctgatcgctgcaagagctgtggagctgctgggaaggtcaagcctgcgaggcctgcagaaagggtgggaagcactgaagtacctgggaaatctgattcagtattggggcctggagctgaagcggagagccatcaacctgctggacattagcgcaatcgccgtggctgagggaacagaccgcattatcgatattgtcctgaggactggccgcgcaattcgaaacatcccaaggcgcatccggcagggatttggagcaaccctgctgtgataa

SEQ ID NO: 34 - protein, 857 aa, synthetic construct
  REGION 1..857: note = pGX1042 - Env Clade C tier 2 Du156.12 Amino Acid Sequence
MRVRGIPRNWPQWWTWGILGFWMIIMCKVAGNSWVTVYYGVPVWTEAKTTLFCASDAKAYEKEVHNVWATHACVPTDPNPQEIFLKNVTENFNMWKNDMVDQMHEDIISLWDQSLKPCVKLTPLCVTLNCVTYNNSMNSSATYNNSMNGEIKNCSFNTTTELRDKKQKVYALFYRTDVVPLNNNNNNSEYILINCNTSTITQACPKVSFDPIPIHYCAPAGYAILKCTDKKFNGTGSCNNVSTVQCTHGIKPVVSTQLLLNGSLAEEEIIIKSENLTDNIKTIIVQLNQSIGINCTRPNNNTRKSVRIGPGQTFYATGDIIGDIRQAHCNISRNQWNETLEQVKKKLGEHFHNQTKIKFEPPSGGDLEITTHSFNCRGEFFYCNTADLFTNATKLVNDTENKAVITIPCRIKQIINMWQGVGRAMYAPPIEGNITCNSNITGLLLTRDGGGNVTEINRTEIFRPGGGNMKDNWRNELYKYKVVEIKPLGVAPTGAKRKVVKREKRAVGLGAVLFGFLGAAGSTMGAASITLTAQARQLLSGIVQQQSNLLRAIEAQQHMLQLTVWGIKQLQARVLAIERYLKDQQLLGLWGCSGKLICTTNVPWNSSWSNKSQTDIWNNTTWMQWEREISNYTDTIYRLLEDSQNQQEENEKDLLALDRWQNLWNWFDITNWLWYIKIFIMIVGGLIGLRIIFGVLSIVKRVREGYSPLSFQTLTPTPRGLDRLGRIEEEGGEQDKDRSIRLVNGFLALAWDDLRSLCLFSYHQLRDFILIAARAVELLGRSSLRGLQKGWEALKYLGNLIQYWGLELKRRAINLLDISAIAVAEGTDRIIDIVLRTGRAIRNIPRRIRQGFGATLL

SEQ ID NO: 35 - DNA, 2586 bp, synthetic construct
  misc_feature 1..2586: note = pGX1043 - Env Clade C tier 2 Du172.17 DNA Sequence
  source 1..2586: mol_type = other DNA
atgagagtgatggggattctgaggtcctatcagcagtggtggatctgggggattctgggattctggatgctgatgatttgtaatgtctggggcaacctgtgggtgaccgtctactatggggtgcctgtctggaaggaggccaaaaccacactgttctgcgcttccgacgccaaggctcataaagaggaagtccataacatctgggcaacacacgcctgtgtgccaactgatccaaacccccaggagattgtgctgaagaatgtcaccgaaaacttcaacatgtggaagaacgacatggtggatcagatgcatgaggacatcatttctctgtgggatcagagtctgaagccttgcgtgaaactgacaccactgtgcgtcactctgaactgttctgacgtgaagatcaaaggcacaaatgccacttacaacaacgctacctacaacaacaacaacacaatcagtgacatgaagaactgttcattcaatactaccacagagatcaccgataagaaaaagaaagaatacgcactgttttataagctggacgtggtcgccctggatggaaaagagaccaacagcacaaatagctccgaataccggctgatcaactgcaatactagtgcagtcacccaggcctgtcccaaggtgtcattcgatcctatcccaattcactactgcgcacctgccggctatgccatcctgaagtgtaacaacaagaccttcaacgggactggaccatgcaacaatgtgagcaccgtccagtgtacacatgggatcaagcccgtggtctccacccagctgctgctgaacggatctctggctgaggaagaggtggtcattaggttcgagaatctgacaaacaatgccaagatcattatcgtgcacctgaacgagtccgtcgaaatcaattgcactcgcccaagcaacaataccagaaaatccgtgaggattggccccgggcagactttctttgctaccggcgacattatcggggatatcagacaggcacattgtaacatttctaggaagaaatggaacactaccctgcagcgggtgaaggagaaactgaaggaaaaattccccaacaagactatccagtttgccccttctagtggcggggacctggagattacaactcacagcttcaattgcagaggcgaattcttttactgttatacatccgatctgtttaacagcacatacatgtccaacaatactggaggcgctaatatcaccctgcagtgccggattaagcagattatcagaatgtggcagggagtgggccaggctatgtatgcaccccctatcgccggaaacattacctgtaaatccaatatcaccggactgctgctgacacgcgacggaggaaaggagaaaaacgatactgaaacctttcgaccaggaggaggagacatgcgagataattggcgatctgagctgtacaagtataaagtggtcgaaatcaagccactgggcattgctcccgacaaggcaaaacggagagtggtcgagcgggaaaaaagagcagtggggatcggagccgtcttcctgggctttctgggagcagctggatctaccatgggagcagccagtatgacactgactgtgcaggccaggcagctgctgtcagggatcgtgcagcagcagagcaacctgctgcgcgccattgaggctcagcagcatatgctgcagctgacagtgtgggggatcaagcagctgcagactagggtgctggccattgaacgctacctgaaggaccagcagctgctgggcatctgggggtgctctggaaaactgatttgtaccacagctgtgccttggaacgcatcctggtctaataagagttatgaagagatctggggcaacatgacctggatgcagtgggatagggagatcaacaattacaccaatacaatctactcactgctggaagagagccagaaccagcaggagaagaatgaaaaagacctgctggctctggatagttgggagtcactgtggagctggttcaacatcacaaattggctgtggtacatcaggatcttcatcatcattgtgggcgggctgatcggactgcgcatcattttcgccgtgctgtcaattgtgaaccgagtccggcagggctattcccctctgtcttttcagactctgacccccagccctagagagccagacaggctggggcgcatcgaagaggaaggaggcgaacaggatagagccaggagcgtgcggctggtcaatggattcctggctctggcatgggaggacctgagatccctgtgcctgttttcttaccaccgcctgcgagatctgatcctgattgctgcacgagccgctgcactgctgggacggtcaagcctgtggggactgcagaagggctgggaggccctgaaatacctggggagtctggtgcagtattggggactggaactgaagaaaagtgccatctcactgttcgacgccatcgctattactgtggctgagggcaccgatcggatcattaacatcgtgcagcgaattagccgggcattctacaatatccccaggcgcattagacaggggtttgaagccaccctgcagtgataa

SEQ ID NO: 36 - protein, 860 aa, synthetic construct
  REGION 1..860: note = pGX1043 - Env Clade C tier 2 Du172.17 Amino Acid Sequence
MRVMGILRSYQQWWIWGILGFWMLMICNVWGNLWVTVYYGVPVWKEAKTTLFCASDAKAHKEEVHNIWATHACVPTDPNPQEIVLKNVTENFNMWKNDMVDQMHEDIISLWDQSLKPCVKLTPLCVTLNCSDVKIKGTNATYNNATYNNNNTISDMKNCSFNTTTEITDKKKKEYALFYKLDVVALDGKETNSTNSSEYRLINCNTSAVTQACPKVSFDPIPIHYCAPAGYAILKCNNKTFNGTGPCNNVSTVQCTHGIKPVVSTQLLLNGSLAEEEVVIRFENLTNNAKIIIVHLNESVEINCTRPSNNTRKSVRIGPGQTFFATGDIIGDIRQAHCNISRKKWNTTLQRVKEKLKEKFPNKTIQFAPSSGGDLEITTHSFNCRGEFFYCYTSDLFNSTYMSNNTGGANITLQCRIKQIIRMWQGVGQAMYAPPIAGNITCKSNITGLLLTRDGGKEKNDTETFRPGGGDMRDNWRSELYKYKVVEIKPLGIAPDKAKRRVVEREKRAVGIGAVFLGFLGAAGSTMGAASMTLTVQARQLLSGIVQQQSNLLRAIEAQQHMLQLTVWGIKQLQTRVLAIERYLKDQQLLGIWGCSGKLICTTAVPWNASWSNKSYEEIWGNMTWMQWDREINNYTNTIYSLLEESQNQQEKNEKDLLALDSWESLWSWFNITNWLWYIRIFIIIVGGLIGLRIIFAVLSIVNRVRQGYSPLSFQTLTPSPREPDRLGRIEEEGGEQDRARSVRLVNGFLALAWEDLRSLCLFSYHRLRDLILIAARAAALLGRSSLWGLQKGWEALKYLGSLVQYWGLELKKSAISLFDAIAITVAEGTDRIINIVQRISRAFYNIPRRIRQGFEATLQ

SEQ ID NO: 37 - DNA, 2538 bp, synthetic construct
  misc_feature 1..2538: note = pGX1018 - Env Clade C tier 2 Cap45.2.00.G3 DNA Sequence
  source 1..2538: mol_type = other DNA
atgcgcgtcagggggattctgcgaaactggcctcagtggtggatctggtctattctgggcttctggatgctgattatctgtagggtcatgggaaacctgtgggtgactgtctactatggcgtgcctgtctggaaagaggctaaggcaaccctgttctgcgcaagcgacgcccgggcttacgaaaaagaggtgcacaacgtctgggcaacccatgcctgcgtgccaacagatcctaacccacaggaaatctatctgggaaatgtgacagagaacttcaacatgtggaagaatgacatggtggatcagatgcacgaggacatcatttcactgtgggatcagagcctgaaaccatgcgtgaagctgacacccctgtgcgtcactctgagatgtactaacgccaccatcaatggctcactgaccgaggaagtgaagaactgtagcttcaatattaccacagagctgcgcgacaagaaacagaaagcatacgccctgttttatcgacctgatgtggtcccactgaataagaactccccctctggcaacagctccgagtacattctgatcaattgcaacacttccaccatcacacaggcctgtcccaaggtgtctttcgaccccattcctatccactactgcgctcctgcagggtatgctatcctgaaatgtaacaacaagaccttcaacgggactggaccatgcaacaacgtgagcaccgtccagtgtacacatggcatcaaacccgtggtctctacccagctgctgctgaatgggagtctggccgaggaagatatcattatcaagtctgagaacctgaccaacaatatcaaaacaattatcgtgcacctgaataagtctgtggaaattgtctgccggagacctaacaataacacacggaagagtattagaatcggcccagggcaggctttctatgcaactaacgacattatcggcgatatcaggcaggcccattgtaacattaataactccacttggaatcgcaccctggaacagatcaagaaaaagctgcgagagcacttcctgaatcggaccattgaatttgagccccctagtggcggggacctggaagtgactacccattcattcaactgcggaggcgagttcttttactgtaacacaactaggctgtttaaatggtctagtaatgtgactaacgatactattaccatcccctgccggatcaagcagttcattaacatgtggcagggagccggcagagctatgtatgcaccacccatcgaggggaacattacctgtaattcaagcatcactggactgctgctgacccgcgacgggggaaaaacagaccgaaacgatactgagatttttcggcctggcgggggaaacatgaaggataactggagaaacgaactgtacaagtacaaggtggtcgagatcaagccactgggagtggctcctaccgaggcaaggcgccgagtggtcgaacgagagaagcgagcagtgggaatcggagctgtcctgctgggcttcctgggagcagctggaagtacaatgggagcagcctcaatcacactgactgtgcaggccaggcagctgctgagcggcatcgtccagcagcagtccaatctgctgcgcgccattgaggctcagcagcacatgctgcagctgacagtgtggggcatcaaacagctgcagactagagtgctggccattgaaaggtacctgaaagaccagcagctgctgggactgtggggatgctctggaaagctgatctgtaccacaaacgtgccatggaattcctcttggagtaacaagtcacagactgacatttgggataatatgacctggattcagtgggatcgggaaatcagcaactactccaacacaatctataaactgctggaggggagccagaaccagcaggaacagaatgagaaggacctgctggccctggatagctggaataacctgtggaattggttcaacatcaccaattggctgtggtacatcaagatctttattatgatcatcggcggactgatcgggctgaggattatcctgggagtgctgagcattgtgaagcgggtcagacagggctattctcctctgagtttccagaccctgacaccaaacccccgcggactggatagactgggcaggatcgaggaagagggaggcgagcaggacaaggatcgcagcattcgactggtgaatgggtttctggccctggcttgggaagacctgcggtccctgtgcctgttctcttaccataggctgcgcgacttcatcctgattgcagtgagagccgtcgaactgctgggaagttcaagcctgaggggactgcagcgaggatgggaggcactgaagtacctgggcagcctgctgcagtattgggggctggaactgaaaaagtccgctatcaacctgctggacaccgtggcaattgccgtcgctgaaggcacagatagaattatcgagctgatccagaggatttgtcgcgctatccgcaatatcccccgccgcatccgccagggctttgaagccgctctgctgtgataa

SEQ ID NO: 38 - protein, 844 aa, synthetic construct
  REGION 1..844: note = pGX1018 - Env Clade C tier 2 Cap45.2.00.G3 Amino Acid Sequence
MRVRGILRNWPQWWIWSILGFWMLIICRVMGNLWVTVYYGVPVWKEAKATLFCASDARAYEKEVHNVWATHACVPTDPNPQEIYLGNVTENFNMWKNDMVDQMHEDIISLWDQSLKPCVKLTPLCVTLRCTNATINGSLTEEVKNCSFNITTELRDKKQKAYALFYRPDVVPLNKNSPSGNSSEYILINCNTSTITQACPKVSFDPIPIHYCAPAGYAILKCNNKTFNGTGPCNNVSTVQCTHGIKPVVSTQLLLNGSLAEEDIIIKSENLTNNIKTIIVHLNKSVEIVCRRPNNNTRKSIRIGPGQAFYATNDIIGDIRQAHCNINNSTWNRTLEQIKKKLREHFLNRTIEFEPPSGGDLEVTTHSFNCGGEFFYCNTTRLFKWSSNVTNDTITIPCRIKQFINMWQGAGRAMYAPPIEGNITCNSSITGLLLTRDGGKTDRNDTEIFRPGGGNMKDNWRNELYKYKVVEIKPLGVAPTEARRRVVEREKRAVGIGAVLLGFLGAAGSTMGAASITLTVQARQLLSGIVQQQSNLLRAIEAQQHMLQLTVWGIKQLQTRVLAIERYLKDQQLLGLWGCSGKLICTTNVPWNSSWSNKSQTDIWDNMTWIQWDREISNYSNTIYKLLEGSQNQQEQNEKDLLALDSWNNLWNWFNITNWLWYIKIFIMIIGGLIGLRIILGVLSIVKRVRQGYSPLSFQTLTPNPRGLDRLGRIEEEGGEQDKDRSIRLVNGFLALAWEDLRSLCLFSYHRLRDFILIAVRAVELLGSSSLRGLQRGWEALKYLGSLLQYWGLELKKSAINLLDTVAIAVAEGTDRIIELIQRICRAIRNIPRRIRQGFEAALL

SEQ ID NO: 39 - DNA, 2511 bp, synthetic construct
  misc_feature 1..2511: note = pGX1022 - Env Clade C tier 2 ZM233M.PB6 DNA Sequence
  source 1..2511: mol_type = other DNA
atgcgcgtgcgggggattatgaggaactggcagcagtggtggatctggggaagtctgggattctggatgctgattatctgtaacgtgatggggtccctgtgggtgacagtctactatggagtgcctgtctggagggaggccaagaccacactgttctgcgctagcgatgctaaagcatacgagactgaagcccactccgtgtgggcaacacatgcctgcgtgccaactgacccaaatccccaggagatggtgctggaaaacgtcacagagaacttcaacatgtggaagaacgacatggtggatcagatgcacgaggacgtgatctctatttgggatcagagtctgaagccttgcgtgaaactgaccccactgtgcgtcacactggattgtagcacatacaacaacactcataacatcagcaaggaaatgaagatctgttccttcaacatgactaccgagctgagggataagaaacgcaaagtgaatgtcctgttttacaaactggacctggtgcccctgaccaatagctccaacacaactaattatcggctgatcagctgcaacacctccacaattactcaggcttgtcccaaggtgagtttcgatcctatcccaattcactactgcgcccctgctggctatgcaatcctgaagtgtaacaacaagaccttcaacgggacaggaccatgcaacaacgtgagcactgtccagtgtacccatggcatcaagcccgtggtctcaactcagctgctgctgaacgggagcctggccgaggaagagatcattatcaggttcgaaaacctgaccgacaatgtgaagattatcattgtccagctgaacgagacaatcaatattacctgcacacgcccaaacaataacactcgaaaatccatccggattggccccgggcagtctttttacgccacaggcgaaatcgtggggaacattagagaggctcactgtaatatctctgcatccaagtggaacaaaaccctggaaagagtgaggacaaagctgaaagagcacttccccaataagaccatcgagtttgaaccttctagtggcggggacctggaaattaccacacattccttcaattgcggaggcgagttcttttactgtaacacctcaggactgtttaacagcgccatcaatggcactctgacctctaatgtgacactgccctgccggattaagcagatcattaacatgtggcaggaagtgggcagagctatgtatgcaccccctatcgctgggaacattacctgtaaatccaatatcactggactgctgctgaccagggatgggggagaaaactcaagctccactaccgagacattccgacctactggcggggacatgaagaataactggagaagcgaactgtacaagtataaagtggtcgagatcaaaccactgggcattgcacccaccgaggcaaagcgaagagtggtcgagcgagaaaaaagagcagtgggaatcggcgccgtcttcctggggtttctgggagccgctggcagtacaatgggggcagcctcaatgacactgactgtgcaggcccgccagctgctgtctggaatcgtgcagcagcagagtaacctgctgaaggccattgaagctcagcagcacatgctgcagctgaccgtgtggggcatcaaacagctgcaggctcgcgtgctggcaattgagcgatacctgaaggatcagcagctgctggggctgtggggatgctcaggcaaactgatctgtacaactaacgtgccatggaatgcctcatggagcaacaagagcaaaaatgacatttgggataatatgacatggatgcagtgggacagggaaatctctaaccataccgatacaatctaccgcctgctggaggacagtcagaaccagcaggagaagaatgaaaaagacctgctggccctggatagttggaagaacctgtggaattggttctcaatcaccaagtggctgtggtacatcaaaatcttcatcatgattgtgggaggcctgatcggcctgcggatcattttcgctgtgctgtccattgtgaatcgcgtccgacagggatattcccctctgtcttttcagactctgacccccaaccctagaggcccagataggctgggcggcatcgaagaggaaggcggggagcaggacaagaacaaaagcaggcgcctggtgactggcttcctgcctgtggtctgggacgatctgagatccctgtgcctgttctcttaccacctgctgagggactttatcctgattgtggcacgaaccgtcgaactgctggggcgacggggatgggaggccctgaagtacctgggaggcctggtgcagtattggggcctggagctgaagaaaagtactatctcactgctggataccatcgccattgtggtcgctgaagggaccgaccggatcattgaggtgctgcagagaatcggccgagccatctacaatatcccaagacgcattcgccagggatttgagaccgctctgctgtgataa

SEQ ID NO: 40 - protein, 835 aa, synthetic construct
  REGION 1..835: note = pGX1022 - Env Clade C tier 2 ZM233M.PB6 Amino Acid Sequence
MRVRGIMRNWQQWWIWGSLGFWMLIICNVMGSLWVTVYYGVPVWREAKTTLFCASDAKAYETEAHSVWATHACVPTDPNPQEMVLENVTENFNMWKNDMVDQMHEDVISIWDQSLKPCVKLTPLCVTLDCSTYNNTHNISKEMKICSFNMTTELRDKKRKVNVLFYKLDLVPLTNSSNTTNYRLISCNTSTITQACPKVSFDPIPIHYCAPAGYAILKCNNKTFNGTGPCNNVSTVQCTHGIKPVVSTQLLLNGSLAEEEIIIRFENLTDNVKIIIVQLNETINITCTRPNNNTRKSIRIGPGQSFYATGEIVGNIREAHCNISASKWNKTLERVRTKLKEHFPNKTIEFEPSSGGDLEITTHSFNCGGEFFYCNTSGLFNSAINGTLTSNVTLPCRIKQIINMWQEVGRAMYAPPIAGNITCKSNITGLLLTRDGGENSSSTTETFRPTGGDMKNNWRSELYKYKVVEIKPLGIAPTEAKRRVVEREKRAVGIGAVFLGFLGAAGSTMGAASMTLTVQARQLLSGIVQQQSNLLKAIEAQQHMLQLTVWGIKQLQARVLAIERYLKDQQLLGLWGCSGKLICTTNVPWNASWSNKSKNDIWDNMTWMQWDREISNHTDTIYRLLEDSQNQQEKNEKDLLALDSWKNLWNWFSITKWLWYIKIFIMIVGGLIGLRIIFAVLSIVNRVRQGYSPLSFQTLTPNPRGPDRLGGIEEEGGEQDKNKSRRLVTGFLPVVWDDLRSLCLFSYHLLRDFILIVARTVELLGRRGWEALKYLGGLVQYWGLELKKSTISLLDTIAIVVAEGTDRIIEVLQRIGRAIYNIPRRIRQGFETALL

SEQ ID NO: 41 - DNA, 2565 bp, synthetic construct
  misc_feature 1..2565: note = pGX1023 - Env Clade C tier 2 ZM249M.PL1 DNA Sequence
  source 1..2565: mol_type = other DNA
atgagagtgatggggattctgaggaactgtcagccctggtggatctggagtattctgggattctggatgctgatgaactgtagcggcaacctgtgggtgaccgtctactatggcgtgcctgtctggagggaggccaagaccacactgttctgcgctagcgacgccaaggcttacgaaaaagaggtgcacaacgtgtgggtcacccatgcctgcgtgccaacagatccaaacccccaggaaatgaatctggagaacgtgacagaaaacttcaacatgtggaaaaacgacatggtggatcagatgcacgaggacatcattagcctgtgggatcagtccctgaagccttgcgtgaaactgacaccactgtgcgtcactctgaactgtaacaatgtgaatgtcacacataactcaacttacaacaataccgaaggggagcagatcaagaattgtagcttcaacattactaccgagctgcgggacaagaaacagaaggtgtacgccctgttttataaactggacatcctgcccctgaatggaaacaatgatagcaacgaatatagactgatcaattgcaacacaagcgccattactcaggcatgtcccaaagtgtccttcgatcctatcccaattcactactgcgcacctgccggctatgctatcctgaagtgtaacaacaagaccttcaacggaaagggcccatgcaacaacgtgagcaccgtccagtgtacacatggcatcaagcccgtggtctccacccagctgctgctgaacggctctctggccgaaaaggagatcattatcaggagtgagaacatcacagacaacgtgaagatcatcatcgtccacctgaatgaatccgtggagattaactgcactcgcccaaacaataacaccaggaagtctatccgcattgggcccggacagactttctacgcaaccggggagatcattggaaagatccgggaagcccattgtaatatttccaaggagaaatggaacaaaaccctgctgcgagtggctaagaaactgcgggaacacttccccggaaaggcaatcaaatttgagcctagctccggcggggacctggaaattacaactcatagcttcaattgcagaggcgagttcttttactgtaccacatctaagctgtttaacagtacatacaaccccaacgatactgagtctaatagtaataacagcaacgaaacactgactctgacctgcaagatcaaacagatcattaatatgtggcagggagtgggacgagcaatgtatgctccccctatcgaggggtcaattacctgtaacagcacaatcactggactgctgctgaccagagacggaggcagcaagaataacacagaggaaatcttccggcctgggggaggcaatatgaaagataactggcgctccgagctgtacaagtataaagtggtcgaaatcaagccactgggagtggcaccaactgaggctaagcgaagagtggtcgaacgcgagaaacgagctgtgggactgggcgcagtcttcctggggtttctgggagcagctggctccacaatgggagcagcctctatcaccctgacagtgcaggccaggcagctgctgtctggaatcgtccagcagcagaataacctgctgcgcgcaattgaggcccagcagcacatgctgcagctgaccgtgtggggcatcaagcagctgcaggcaagagtcctggccattgaaaggtacctgaaggaccagcagctgctggggatctggggatgcagtggcaaactgatttgtactacctcagtgccctggaatacatcatggagcaacaagagtaaagccgagatctgggacaacatgacttggatgcagtgggataaggaaatctcaaattacactcagaccatctacaacctgctggaggaatcccagtctcagcaggaaaagaatgagaaagacctgctggagctggattcttggaataacctgtggaactggttcgacatcagtaagtggctgtggtacatcaaaatcttcatcatgattgtgggcggcctgatcggcctgaggatcattttcgccgtgctgtccattgtgaatagggtccgccaggggtatagtcctctgtcatttcagatcctgaccccaaaccctcgcggaccagatcgactgggcagaattgaggaagagggcggggagcaggaccgagatcggtctgtgcgactggccaatgggttcctggctctggcatgggaagacctgagaaacctgtgcctgttcttttaccacagactgagggatttcatcctgattgctgcacgcacagtggagctgctgcgacagatcagctttaagggcctgcagcgggggtgggaagctctgaaatacctgggcagtctggtgcagtattggtcacaggaactgaaggagagcgccatcaatctgctgaacactatcgccattgctgtggcagagggcaccgatcggatcattgaagtggtccagagagggtttcgcgccatcctgaatgtccccacccgcatccgccagggcctggagagagcactgctgtgataa

SEQ ID NO: 42 - protein, 853 aa, synthetic construct
  REGION 1..853: note = pGX1023 - Env Clade C tier 2 ZM249M.PL1 Amino Acid Sequence
MRVMGILRNCQPWWIWSILGFWMLMNCSGNLWVTVYYGVPVWREAKTTLFCASDAKAYEKEVHNVWVTHACVPTDPNPQEMNLENVTENFNMWKNDMVDQMHEDIISLWDQSLKPCVKLTPLCVTLNCNNVNVTHNSTYNNTEGEQIKNCSFNITTELRDKKQKVYALFYKLDILPLNGNNDSNEYRLINCNTSAITQACPKVSFDPIPIHYCAPAGYAILKCNNKTFNGKGPCNNVSTVQCTHGIKPVVSTQLLLNGSLAEKEIIIRSENITDNVKIIIVHLNESVEINCTRPNNNTRKSIRIGPGQTFYATGEIIGKIREAHCNISKEKWNKTLLRVAKKLREHFPGKAIKFEPSSGGDLEITTHSFNCRGEFFYCTTSKLFNSTYNPNDTESNSNNSNETLTLTCKIKQIINMWQGVGRAMYAPPIEGSITCNSTITGLLLTRDGGSKNNTEEIFRPGGGNMKDNWRSELYKYKVVEIKPLGVAPTEAKRRVVEREKRAVGLGAVFLGFLGAAGSTMGAASITLTVQARQLLSGIVQQQNNLLRAIEAQQHMLQLTVWGIKQLQARVLAIERYLKDQQLLGIWGCSGKLICTTSVPWNTSWSNKSKAEIWDNMTWMQWDKEISNYTQTIYNLLEESQSQQEKNEKDLLELDSWNNLWNWFDISKWLWYIKIFIMIVGGLIGLRIIFAVLSIVNRVRQGYSPLSFQILTPNPRGPDRLGRIEEEGGEQDRDRSVRLANGFLALAWEDLRNLCLFFYHRLRDFILIAARTVELLRQISFKGLQRGWEALKYLGSLVQYWSQELKESAINLLNTIAIAVAEGTDRIIEVVQRGFRAILNVPTRIRQGLERALL

SEQ ID NO: 43 - DNA, 2571 bp, synthetic construct
  misc_feature 1..2571: note = pGX1024 - Env Clade C tier 2 ZM214M.PL15 DNA Sequence
  source 1..2571: mol_type = other DNA
atgcgcgtgagggggatgctgcgaaactgtcagcagtggtggatctgggggattctgggcttttggatgctgatgatttgtaacggggtgggcaacctgtgggtgacagtctactatggggtgcccgtctggagggaggcaaagaccacactgttttgcgcctccgacgccaaggcttacgaaaaagaggtgcacaatgtctgggccacccatgcttgcgtgcctacagatccaaacccccaggaactggtgctggagaatgtcaccgaaaacttcaatatgtggaagaacgacatggtgaatcagatgcacgaggacatcattagtctgtgggatcagtcactgaagccttgcgtgaaactgaccccactgtgcgtcacactgaactgtagtaacgtgaacatcaacgaaacatcaatcgatttcaacgtcactagcaatatctccatgaaggaggaaatgaagaactgtagctttaaggtgaactccgagctgagggacaaaaatcggagagaacatgccctgttctataagctggatatcgtgcagctgaacgacgagggcaatgattcatacagctatcgcctgattaattgcaacacctctacaatcaagcaggcttgtccaaaagtgagttttgagcctatcccaattcactactgcgcacccgccggctatgcaatcctgaagtgtaacaatgaaacattcaacggcagcggcccttgcaacaacgtgagcaccgtccagtgtacacatggaatcaaaccagtggtcagcactcagctgctgctgaacggctccctggccgaaaaggagatcatgattaggtccgagaatctgactaacaatgctaaaaccatcattgtgcagctgactgaagcagtcaacattacctgcatgcgacccggcaacaataccaggcgcagtgtgcggatcggacctggacagactttttacgccaccggggagatcattggagacattcggcaggctcactgtaatatcagcaaggataaatggaaccagatcctgcagaatgtgagagccaagctgggcgagcacttccatgacaagaccatcaagtttgagccaagctccggcggggatctggaaatcactacccattctttcaactgcggaggcgaattcttttactgtaacacaactaatctgttttcccgcacttataccaatggctccaattctaacgtgaatattacctctgccacaatcactctgccctgccgcattaagcagatcattaacatgtggcaggaagtgggacgagcaatgtatgcccctcccatcgctggcaacatcacttgtattagcaatatcacaggactgctgctgactcgggacgggggaaacggaaatgacaccaacgataccgagacattcagacctgccggcggggacatgagagataattggaggagcgagctgtacaagtataaagtggtcgaaattaagccactgggcatcgcccccaccaaggctaaacgacgagtggtcggaagggagaaacgagcagtgggcattggggctgtcttcctgggatttctgggagcagctgggtcaacaatgggagcagccagcatcactctgaccgtccaggcaaggcagctgctgagcggaattgtgcagcagcagaacaatctgctgcgcgctatcgaggcacagcagcacctgctgcagctgaccgtctggggcattaagcagctgcaggcacgcgtgctggccatcgaacgatacctgaaggatcagcagctgctgggactgtggggctgctcagggaaactgatctgtaccacaactgtcagctggaactctagttggtctaacaagagtgtggacgatatttggcagaacatgacctggatgcagtgggacagagagatcaacaattacacagaaatcatctacaggctgctggaggtgagccagaaccagcaggaaaagaatgaggaagacctgctggccctggacaaatgggataacctgtggaattggttcgatatctccaagtggctgtggtacatcaaaatcttcatcatgattgtcggaggcctgattggcctgcggatcatttttgctgtgctgtctatcgtgaaccgcgtccgacaggggtattcacccctgagcttccagacactgactcccaatcctagagagctggaccgactgggacggattgaggaagagggcggcgagcaggatcggagtagatcaatcaggctggtgaacggcttcctggctctggcatgggacgatctgcgctctctgtgcctgtttagttaccaccatctgagggacctgatcctgattgctgcacgcactgtgagcctgctgggaagaaggggctgggaggcactgaagtacctgggcgggctggtgcagtattgggggagagaactgaagaaatccgccatttctctgctggacacagtggctatcactgtcgcagagggcaccgatagagtgatcgaaattgcccagagattcggaagaggaatctgtaatatcccccgacgaatccgccagggctttgaagccgctctgcagtgataa

SEQ ID NO: 44 - protein, 855 aa, synthetic construct
  REGION 1..855: note = pGX1024 - Env Clade C tier 2 ZM214M.PL15 Amino Acid Sequence
MRVRGMLRNCQQWWIWGILGFWMLMICNGVGNLWVTVYYGVPVWREAKTTLFCASDAKAYEKEVHNVWATHACVPTDPNPQELVLENVTENFNMWKNDMVNQMHEDIISLWDQSLKPCVKLTPLCVTLNCSNVNINETSIDFNVTSNISMKEEMKNCSFKVNSELRDKNRREHALFYKLDIVQLNDEGNDSYSYRLINCNTSTIKQACPKVSFEPIPIHYCAPAGYAILKCNNETFNGSGPCNNVSTVQCTHGIKPVVSTQLLLNGSLAEKEIMIRSENLTNNAKTIIVQLTEAVNITCMRPGNNTRRSVRIGPGQTFYATGEIIGDIRQAHCNISKDKWNQILQNVRAKLGEHFHDKTIKFEPSSGGDLEITTHSFNCGGEFFYCNTTNLFSRTYTNGSNSNVNITSATITLPCRIKQIINMWQEVGRAMYAPPIAGNITCISNITGLLLTRDGGNGNDTNDTETFRPAGGDMRDNWRSELYKYKVVEIKPLGIAPTKAKRRVVGREKRAVGIGAVFLGFLGAAGSTMGAASITLTVQARQLLSGIVQQQNNLLRAIEAQQHLLQLTVWGIKQLQARVLAIERYLKDQQLLGLWGCSGKLICTTTVSWNSSWSNKSVDDIWQNMTWMQWDREINNYTEIIYRLLEVSQNQQEKNEEDLLALDKWDNLWNWFDISKWLWYIKIFIMIVGGLIGLRIIFAVLSIVNRVRQGYSPLSFQTLTPNPRELDRLGRIEEEGGEQDRSRSIRLVNGFLALAWDDLRSLCLFSYHHLRDLILIAARTVSLLGRRGWEALKYLGGLVQYWGRELKKSAISLLDTVAITVAEGTDRVIEIAQRFGRGICNIPRRIRQGFEAALQ

SEQ ID NO: 45 - DNA, 2565 bp, synthetic construct
  misc_feature 1..2565: note = pGX1029 Env Clade A tier 2 Q23ENV17 DNA sequence
  source 1..2565: mol_type = other DNA
atgagagtgatgggcattcagaggaactgtcagcacctgctgacctggggcattatgattctggggactattatcttttgtagcgcagtggagaacctgtgggtgactgtctactatggagtgccagtctggcgagacgcagataccacactgttctgcgctagcgacgctaaggcatacgaaacagagaaacacaacgtgtgggcaacccatgcctgcgtgcccacagacccaaatccccaggaaatccacctggataatgtcacagagaagtttaacatgtggaagaacaacatggtggagcagatgcatactgacatcatttctctgtgggatcagagtctgaagccttgcgtgaaactgactccactgtgcgtcaccctgcactgtacaaatgtgacttccgtcaacactaccggcgacagagaagggctgaagaattgttctttcaacatgacaactgagctgcgggacaagagacagaaagtctacagcctgttttatcggctggatatcgtgcccattaatgaaaaccagggcagtgagtacagactgatcaattgcaacacttcagctattacccaggcatgtccaaaggtgagcttcgagcctatcccaattcactattgcacccccgctggcttcgcaatcctgaagtgtaaagatgaagggtttaatggaacaggcctgtgcaaaaacgtgtctacagtccagtgtactcatgggattaagcctgtggtctcaacccagctgctgctgaatggaagcctggccgagaagaacatcaccattaggagtgagaacatcacaaacaacgctaagatcatcatcgtgcagctggtccagcccgtgaccatcaaatgcattcgccctaacaataacacacgcaagagcatccgaattgggccaggacaggccttttacgctaccggagacattatcggcgatatccggcaggcccactgtaacgtgactaggtcccgctggaataagaccctgcaggaagtggccgagaaactgagaacttatttcggcaacaagaccattatctttgccaatagctccggcggggacctggaaatcaccacacatagtttcaactgcggaggcgagttcttttactgtaatacctcagggctgtttaacagcacatggtacgtgaattcaacttggaacgacaccgatagcacacaggagtccaacgatacaatcactctgccctgccgaattaagcagattatcaatatgtggcagcgagcaggacaggcaatgtacgctccacctatccctggcgtgatcaagtgtgagagcaacatcacagggctgctgctgactagagacgggggaaaggataataacgtgaacgagaccttcaggccaggaggaggagacatgcgagataattggagaagcgaactgtacaagtataaagtggtcgaaatcgagccactgggagtggcaccaacaagggctaaacggagagtggtcgaaagggagaagcgagctgtgggaatcggagcagtcttcctggggtttctgggagccgctggctctaccatgggcgcaacaagtattaccctgacagtccaggctaggcagctgctgtccgggatcgtgcagcagcagaataacctgctgcgcgcaattgaggcccagcagcacctgctgaagctgaccgtgtggggcatcaaacagctgcaggcaagggtcctggcagtggagcgatatctgcgagaccagcagctgctgggaatctggggatgctccggcaaactgatttgtactaccaatgtgccttggaactctagttggtccaacaagtctctggacgaaatctggaataacatgacttggctgcagtgggataaagagattaataactacacccagctgatctatcgcctgattgaggaatctcagaatcagcaggaaaagaacgaaaaagagctgctggagctggacaagtgggccaacctgtggtcctggttcgatatttctaattggctgtggtacatcaagatcttcatcatcattgtgggcgggctgatcggactgcggattgtcttcgccgtgctgtctgtcatcaaccgagtgcggcagggctatagtcctctgtcatttcagactcatacccccaatcctagaggactggacagaccagaaaggatcgaggaagaggatggcgagcagggaagaggcaggagtattcgcctggtgtcaggcttcctggccctggcttgggacgatctgcgaagcctgtgcctgttctcctaccaccgcctgcgagacttcatcctgattgcagccaggaccgtggaactgctggggcattcaagcctgaaaggactgcgcctggggtgggagggaatcaagtacctgtggaacctgctgtcctattgggggcgggaactgaagatctctgccattaatctggtggacacaatcgcaattgccgtcgctggatggactgatagagtgatcgagattgcccagcgcatcggaagagctattctgcatatccccgtgaggattcgccagggactggaaagagcactgctgtgataa

SEQ ID NO: 46 - protein, 853 aa, synthetic construct
  REGION 1..853: note = pGX1029 Env Clade A tier 2 Q23ENV17 Amino Acid Sequence
MRVMGIQRNCQHLLTWGIMILGTIIFCSAVENLWVTVYYGVPVWRDADTTLFCASDAKAYETEKHNVWATHACVPTDPNPQEIHLDNVTEKFNMWKNNMVEQMHTDIISLWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNITIRSENITNNAKIIIVQLVQPVTIKCIRPNNNTRKSIRIGPGQAFYATGDIIGDIRQAHCNVTRSRWNKTLQEVAEKLRTYFGNKTIIFANSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGGDMRDNWRSELYKYKVVEIEPLGVAPTRAKRRVVEREKRAVGIGAVFLGFLGAAGSTMGATSITLTVQARQLLSGIVQQQNNLLRAIEAQQHLLKLTVWGIKQLQARVLAVERYLRDQQLLGIWGCSGKLICTTNVPWNSSWSNKSLDEIWNNMTWLQWDKEINNYTQLIYRLIEESQNQQEKNEKELLELDKWANLWSWFDISNWLWYIKIFIIIVGGLIGLRIVFAVLSVINRVRQGYSPLSFQTHTPNPRGLDRPERIEEEDGEQGRGRSIRLVSGFLALAWDDLRSLCLFSYHRLRDFILIAARTVELLGHSSLKGLRLGWEGIKYLWNLLSYWGRELKISAINLVDTIAIAVAGWTDRVIEIAQRIGRAILHIPVRIRQGLERALL

SEQ ID NO: 47 - DNA, 2313 bp, synthetic construct
  misc_feature 1..2313: note = pGX1004 MPol DNA sequence
  source 1..2313: mol_type = other DNA
atggactggacctggattctgttcctggtggccgctgccaccagagtgcacagccctcagatcaccctgtggcagagacctctggtgaccatcaagatcggcggccagctgaaggaggccctgctggccgacgacaccgtgctggaggagatcaacctgcccggcaagtggaagcctaagatgatcggcggcatcgggggcttcatcaaagtgaggcagtacgaccagatcctgatcgagatctgtggccacaaggccatcggcacagtgctggtcggccccacacccgtgaatatcatcggccggaacatgctgacccagatcggctgtaccctgaacttccccatcagccccatcgagaccgtgcctgtgaagctgaagcctggcatggatggccctaaggtgaagcagtggcccctgaccgaggagaagatcaaggccctgacagagatctgtaccgagatggagaaggagggcaagatcagcaagatcggccccgagaacccctacaacacccccgtgttcgccatcaagaagaaggacagcaccaagtggcggaaactggtggacttccgggagctgaacaagaggacccaggacttctgggaggtgcagctgggcatccctcaccctgccggcctgaagaagaagaagtccgtgacagtgctggatgtgggcgacgcctacttcagcgtgcccctggacgaggacttcaggaagtacaccgccttcaccatccccagcatcaacaacgagacccccggcatcagataccagtacaacgtgctgcctcagggctggaagggcagccccgccatcttccagagcagcatgaccaagatcctggagcccttcaggaagcagaaccccgagatcgtgatctaccagctgtatgtgggcagcgatctggagatcggccagcacagagccaagatcgaggagctgagggagcacctgctgagatggggcttcaccacccccgataagaagcaccagaaggagccccctttcctgtggatgggctacgagctgcaccctgacaagtggaccgtgcagcccatcaagctgcctgagaaggagagctggaccgtgaacgacatccagaaactggtgggcaagctgaattgggccagccagatctacgccggcattaaagtgagacagctgtgtaagctgctgagaggcgccaaagccctgaccgaagtggtgcctctgacagaggaggccgagctggagctggccgagaacagggagatcctgaaggagcccgtgcacggcgtgtactacgaccccagcaaggatctgatcgccgagatccagaagcagggccagggccagtggacctaccagatctaccaggagcctttcaagaacctgaaaaccggcaagtacgccagaatgaggggagcccacaccaacgatgtgaagcagctgaccgaggccgtgcagaaaatcgccatggagagcatcgtgatctggggcaagacacccaagttccggctgcccatccagaaggagacctgggaaacctggtggaccgagtactggcaggccacctggattcctgagtgggagttcgtgaacaccccccctctggtgaagctgtggtatcagctggagaaggaacctatcgccggagccgagaccttctacgtggacggagccgccaatagagagaccaagctgggcaaggccggctacgtgaccgacagaggcagacagaaggtggtgtccctgaccgacaccaccaaccagaaaaccctgcaggccatccacctggccctgcaggacagcggcctggaggtgaacatcgtgaccgactcccagtacgccctgggcatcatccaggcccagcccgacaagagcgagagcgagctggtgtcccagatcatcgagcagctgatcaagaaggagaaggtgtacctgagctgggtgcccgcccacaagggcattggcggcaatgagcaggtggacaagctggtgtctagcggcatccggaaggtgctgtacccctacgacgtgcccgattacgcctgagaattcgtaagtaagtgtcatatgggagagctcgactagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacatgtccggagccgtgctgcccggatgatgtcttggcctctgtttgctgcggccgc

SEQ ID NO: 48 - protein, 678 aa, synthetic construct
  REGION 1..678: note = pGX 1004 MPol Protein sequence
MDWTWILFLVAAATRVHSPQITLWQRPLVTIKIGGQLKEALLADDTVLEEINLPGKWKPKMIGGIGGFIKVRQYDQILIEICGHKAIGTVLVGPTPVNIIGRNMLTQIGCTLNFPISPIETVPVKLKPGMDGPKVKQWPLTEEKIKALTEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPEIVIYQLYVGSDLEIGQHRAKIEELREHLLRWGFTTPDKKHQKEPPFLWMGYELHPDKWTVQPIKLPEKESWTVNDIQKLVGKLNWASQIYAGIKVRQLCKLLRGAKALTEVVPLTEEAELELAENREILKEPVHGVYYDPSKDLIAEIQKQGQGQWTYQIYQEPFKNLKTGKYARMRGAHTNDVKQLTEAVQKIAMESIVIWGKTPKFRLPIQKETWETWWTEYWQATWIPEWEFVNTPPLVKLWYQLEKEPIAGAETFYVDGAANRETKLGKAGYVTDRGRQKVVSLTDTTNQKTLQAIHLALQDSGLEVNIVTDSQYALGIIQAQPDKSESELVSQIIEQLIKKEKVYLSWVPAHKGIGGNEQVDKLVSSGIRKVLYPYDVPDYA

SEQ ID NO: 49 - DNA, 2865 bp, synthetic construct
  misc_feature 1..2865: note = pGX1016 pPK2C1 (PrimaryPOL) DNA sequence
  source 1..2865: mol_type = other DNA
atggattggacttggatcttatttttagttgctgctactagagttcgctctcctcagatcacgctctggcagcggccgctcgtcacaataaagatcggggggcaactcaaggaggcgctgctcgcggacgacacggtcttggaggagatgtcgttgccggggcggtggaagccgaagatgatcggggggatcgggggcttcatcaaggtgcggcagtacgaccagatcctcatcgagatctgcgggcacaaggcgatcgggacggtcctcgtcggcccgacgccggtcaacatcatcgggcggaacctgttgacccagatcggctgcaccttgaacttccccatcagccctattgagacggtgcccgtgaagttgaagccggggatggacggccccaaggtcaagcaatggccattgacggaggagaagatcaaggccttagtcgaaatctgtacagagatggagaaggaagggaagatcagcaagatcgggcctgagaacccctacaacactccagtcttcgcaatcaagaagaaggacagtaccaagtggagaaagctggtggacttcagagagctgaacaagagaactcaggacttcggggaagttcagctgggcatcccacatcccgctgggttgaagaagaagaagtcagtgacagtgctggatgtgggtgatgcctacttctccgttcccttggacgaggacttcaggaagtacactgccttcacgatacctagcatcaacaacgagacaccaggcatccgctaccagtacaacgtgctgccacagggatggaagggatcaccagccatctttcaatcgtcgatgaccaagatcctggagcccttccgcaagcaaaacccagacatcgtgatctatcagctctacgtaggaagtgacctggagatcgggcagcacaggaccaagatcgaggagctgagacagcatctgttgaggtggggactgaccaccccagacaagaagcaccagaaggaacctcccttcctgtggatgggctacgaactgcatcctgacaagtggacagtgcagcccatcgtgctgcctgagaaggacagctggactgtgaacgacatacagaagctcgtgggcaagttgaactgggcaagccagatctacccaggcatcaaagttaggcagctgtgcaagctgcttcgaggaaccaaggcactgacagaagtgatcccactgacagaggaagcagagctagaactggcagagaaccgagagatcctgaaggagccagtacatggagtgtactacgacccaagcaaggacctgatcgcagagatccagaagcaggggcaaggccaatggacctaccaaatctaccaggagcccttcaagaacctgaagacaggcaagtacgcaaggatgaggggtgcccacaccaacgatgtgaagcagctgacagaggcagtgcagaagatcaccacagagagcatcgtgatctggggcaagactcccaagttcaagctgcccatacagaaggagacatgggagacatggtggaccgagtactggcaagccacctggatccctgagtgggagttcgtgaacaccccttccctggtgaaactgtggtatcagctggagaaggaacccatcgtgggagcagagaccttctacgtggatggggcagccaacagggagaccaagctgggcaaggcaggctacgtgaccaaccgaggacgacagaaagtggtgaccctgactgacaccaccaaccagaagactctgcaagccatctacctagctctgcaagacagcggactggaagtgaacatcgtgacagactcacagtacgcactgggcatcatccaagcacaaccagaccaatccgagtcagagctggtgaaccagatcatcgagcagctgatcaagaaggagaaagtgtacctggcatgggtcccggcgcacaaggggatcggggggaacgagcaggtcgacaagttggtctcggcggggatccggaaggtgctgttcctggacgggatcgataaggcccaagatgaacatgagaagtaccactccaactggcgcgctatggccagcgacttcaacctgccgccggtcgtcgcgaaggagatcgtcgccagctgcgacaagtgccagctcaagggggaggccatgcacgggcaagtcgactgcagtccggggatctggcagctgtgcacgcacctggaggggaaggtgatcctggtcgcggtccacgtcgccagcgggtatatcgaggcggaggtcatcccggctgagacggggcaggagacggcgtacttcctcttgaagctcgcggggcggtggccggtcaagacgatccacacgaacgggagcaacttcacgggggcgacggtcaaggccgcctgttggtgggcgggaatcaagcaggaatttggaattccctacaatccccaatcgcaaggagtcgtgagcatgaacaaggagctgaagaagatcatcggacaaagggatcaggctgagcacctgaagacagcagtgcagatggcagtgttcatccacaacttcaaaagaaaaggggggattggggggtacagtgcgggggaacggatcgtggacatcatcgccaccgacatccaaaccaaggagctgcagaagcagatcaccaagatccagaacttccgggtgtactaccgcgacagccgcaacccactgtggaagggaccagcaaagctcctctggaagggagagggggcagtggtgatccaggacaacagtgacatcaaagtggtgccaaggcgcaaggccaagatcatccgcgactatggaaaacagatggcaggggatgattgtgtggcaagtagacaggatgaggatggcgcctag

SEQ ID NO: 50 - protein, 954 aa, synthetic construct
  REGION 1..954: note = pGX1016 pPK2C1 (PrimaryPOL) Amino Acid sequence
MDWTWILFLVAATRVRSPQITLWQRPLVTIKIGGQLKEALLADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFGEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPSLVKLWYQLEKEPIVGAETFYVDGAANRETKLGKAGYVTNRGRQKVVTLTDTTNQKTLQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLCTHLEGKVILVAVHVASGYIEAEVIPAETGQETAYFLLKLAGRWPVKTIHTNGSNFTGATVKAACWWAGIKQEFGIPYNPQSQGVVSMNKELKKIIGQRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDEDGA

SEQ ID NO: 51 - DNA, 2565 bp, synthetic construct
  misc_feature 1..2565: note = pGX1053 Env Clade B tier 1B NL43 DNA Sequence
  source 1..2565: mol_type = other DNA
atgagagtgaaggaaaagtaccagcacctgtggagatggggatggaagtgggggactatgctgctggggattctgatgatttgtagcgccaccgaaaagctgtgggtgacagtctactatggcgtgccagtctggaaagaggcaaccacaactctgttctgcgcctccgacgccaaggcttacgatactgaggtgcacaatgtctgggcaactcatgcctgtgtgcccaccgacccaaatccccaggaagtggtcctggtgaacgtcaccgagaattttaacatgtggaagaacgatatggtggaacagatgcacgaggacatcatttcactgtgggatcagagcctgaagccctgcgtgaaactgacacctctgtgcgtcagcctgaagtgtactgacctgaaaaacgatactaataccaacagctcctctggccgcatgatcatggaaaagggagagatcaagaactgtagcttcaatatctctaccagtattagggacaaggtgcagaaagaatacgccttcttttataagctggacatcgtgccaattgataatacatcctacagactgatcagctgcaacacatccgtgattactcaggcatgtccaaaggtctcttttgagcctatcccaattcactattgcgcacccgccggcttcgctatcctgaagtgtaacaacaagacctttaacggaacagggccctgcactaatgtgtccaccgtccagtgtacacatgggatcagacctgtggtcagtacccagctgctgctgaacggctcactggccgaggaagacgtggtcatccggtctgccaacttcactgataatgctaaaaccatcattgtgcagctgaacacatctgtcgagatcaattgcacacgacctaacaataacactaggaagagtatcagaatccagcggggcccaggacgcgcttttgtgacaatcggaaaaattgggaacatgcgccaggcacactgtaatatcagccgagcaaagtggaacgccactctgaagcagattgcctccaaactgagggagcagttcggcaataacaagactatcatcttcaagcagagttcaggcggagaccctgaaatcgtgacccatagcttcaattgcgggggcgagttcttttactgtaacagtacccagctgttcaactcaacatggtttaattccacttggtctaccgaagggagcaataacaccgagggctccgatacaatcactctgccatgccgcatcaagcagttcattaatatgtggcaggaagtggggaaagctatgtatgcaccccctatcagcggccagattaggtgtagctccaacatcacaggactgctgctgactagagatggagggaataataataacggctcagagatctttagacctggcggaggggacatgagggataactggagaagcgaactgtacaagtataaagtggtcaaaatcgagcctctgggagtggctccaacaaaggcaaaaaggagagtggtccagcgagagaagcgagcagtgggaatcggagcactgttcctgggctttctgggagccgctgggtctactatgggggcagccagtatgaccctgacagtgcaggcccgacagctgctgtccgacatcgtccagcagcagaataacctgctgcgggccattgaagctcagcagcacctgctgcagctgaccgtgtggggaatcaagcagctgcaggccagaatcctggctgtggagcggtacctgaaagatcagcagctgctgggcatctggggatgcagtgggaagctgatttgtaccacagctgtgccctggaacgcatcatggagcaataagagcctggagcagatctggaataacatgacctggatggaatgggaccgggagattaataactacacatctctgatccatagtctgattgaggaatcccagaaccagcaggaaaagaatgaacaggagctgctggagctggataaatgggcctctctgtggaattggttcaacatcaccaattggctgtggtacattaagctgtttatcatgattgtgggcggactggtcggactgaggatcgtgttcgctgtcctgtctattgtgaaccgagtcaggcaggggtatagtcctctgtcatttcagacacacctgccaatccctcgaggaccagaccgacccgaagggattgaggaagagggaggagagagagaccgagatcgatccatccggctggtgaacggctctctggccctgatttgggacgatctgcgctccctgtgcctgttctcttaccatcgactgagggatctgctgctgatcgtgaccagaattgtcgaactgctgggacgacgaggatgggaggccctgaaatactggtggaatctgctgcagtattggtcacaggagctgaagaacagcgctgtgaacctgctgaatgctactgcaatcgccgtggctgaaggcaccgacagagtgatcgaggtcctgcaggctgcatatcgggctattaggcacatcccaagacgcattagacaggggctggaacgcatcctgctgtaa

SEQ ID NO: 52 - protein, 854 aa, synthetic construct
  REGION 1..854: note = pGX1053 Env Clade B tier 1B NL43 Amino Acid Sequence
MRVKEKYQHLWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRDKVQKEYAFFYKLDIVPIDNTSYRLISCNTSVITQACPKVSFEPIPIHYCAPAGFAILKCNNKTFNGTGPCTNVSTVQCTHGIRPVVSTQLLLNGSLAEEDVVIRSANFTDNAKTIIVQLNTSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGAASMTLTVQARQLLSDIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 53 - DNA, 2586 bp, synthetic construct
  misc_feature 1..2586: note = pGX1054 Env Clade B tier 2 AC10.0.29 DNA Sequence
  source 1..2586: mol_type = other DNA
atgagagtccgggagacacgaaaaaactatcagcacctgtggtggaaatggggaatgatgctgctgggaatgctgatgatctgttcagccgtggaacagacctgggtgacagtctactatggcgtgccagtctggaaggaggctaacaccacactgttctgcgcaagcgacgctaaagcatacaacacagaggtgcacaatgtctgggcaactcatgcctgtgtgcccaccgatccaaatccccaggaggtggaactggagaacgtcactgaaaacttcaacatgtggaagaacaacatggtggaccagatgcacgaggatatcattagtctgtgggaccagtcactgaagccttgcgtgaaactgaccccactgtgcgtcactctgtcatgtaccgacaacgtggggaatgatactagcaccaacaattcccgctgggataagatggaaaaaggagagatcaagaattgtagcttcaacattactaccaatatgcgggacaagatgcagaaacagtacgccctgttttataagctggatgtggtccccatcgaggaagggaaaaacaataacagctccttcaccgactaccgcctgatctcttgcaatacaagtgtgattactcaggcctgtcctaaggtcacatttgagcctatcccaattcactattgcgccccagctggattcgctctgctgaagtgtaaagataagaagttcaacggcactgggccctgcaagaacgtgagcaccgtccagtgtacacatggcatcaaacctgtggtcagtacccagctgctgctgaacgggtcactggctgaggaagaggtggtcatcagatcagaaaatttcagcaataacgcaaggaccatcattgtgcagctgaacacatccgtcgagatcaagtgcattcggccaaataacaataccagaaaaggcatccacattggacccggccgggcattttacacaactggggacatcattggagatatcaggcaggcccattgtaacatttctcgccagaattggaacaatacactgaagcagatcgccgaaaaactgagagagcagttcgggaataagactatcgtgtttaggaactctagtggcggggaccctgagattgtgatgcacactttcaactgcgcaggagaattcttttactgtaacaccgccgagctgtttaatagcacatggtatgctaacggcactatctccattggaggcgggaacaagaccaatatcattctgccatgcagaatcaaacagttcattaatatgtggcaggaagtgggaaaggctatgtatgcaccccctatcagtggccagattaggtgttcaagcaacatcacaggactgctgctgacccgggacggaggacgaggaaaccagactgataatcagaccgagatcttcagacccgtggggggagatatgaaaaacaattggcgcagcgaactgtacaagtataaagtggtccgaatcgagccactgggaattgcaccaacccgggccaagcgaagagtggtccagcgagagaaaagagccgtggggatcggagctctgttcctgggatttctgggagcagctgggtccacaatgggagcagcctctatgacactgactgtgcaggcccgcctgctgctgtctgggatcgtgcagcagcagaacaatctgctgcgggccattgaagctcagcagcatctgctgcagctgaccgtgtggggcatcaagcagctgcaggctagggtgctggcagtcgagaggtacctgcgcgaccagcagctgctgggaatctggggctgcagcgggaaactgatttgtaccacagccgtgccttggaacgtcagctggaacaatagatccgtggacgatatctgggaaaatatgacatggatgcagtgggacagggagatttccaactacacctctctgatctatacactgattgaagagtcccagaaccagcaggaaaagaatgaacaggagctgctggcactggataaatgggccaacctgtggaattggttcaacatcactgagtggctgtggtacatcaagatttttatcatgattgtgggcgggctggtcggcctgagaatcgtgttcgccgtcctgtccattgtgaatcgagtccggcagggatattcccccctgtcttttcagacacacctgcctgctcagagaggaccagacaggcctggaggaatcgaagaggaagggggagagtctgacagagataggagtggccgcctggtgaacgggttcctggccatcatttggatcgacctgcgatcactgtgcctgtttagctatcaccatctgcgagatctgctgctgattgtgacccggatcgtcgaaattctgggaaggcgcggctgggagatcctgaagtactggtggaacctgctgcagtattggattcaggagctgaaaaatagtgccgtgtcactgctgaacgcaatcgccattgctgtgggcgaagggaaggatcgcatcattgaggccttccgctctatctttcgagctatcctgcatattccaacccgcattcgacagggactggagcgaagtctgctgtgataa

SEQ ID NO: 54 - protein, 860 aa, synthetic construct
  REGION 1..860: note = pGX1054 Env Clade B tier 2 AC10.0.29 Amino Acid Sequence
MRVRETRKNYQHLWWKWGMMLLGMLMICSAVEQTWVTVYYGVPVWKEANTTLFCASDAKAYNTEVHNVWATHACVPTDPNPQEVELENVTENFNMWKNNMVDQMHEDIISLWDQSLKPCVKLTPLCVTLSCTDNVGNDTSTNNSRWDKMEKGEIKNCSFNITTNMRDKMQKQYALFYKLDVVPIEEGKNNNSSFTDYRLISCNTSVITQACPKVTFEPIPIHYCAPAGFALLKCKDKKFNGTGPCKNVSTVQCTHGIKPVVSTQLLLNGSLAEEEVVIRSENFSNNARTIIVQLNTSVEIKCIRPNNNTRKGIHIGPGRAFYTTGDIIGDIRQAHCNISRQNWNNTLKQIAEKLREQFGNKTIVFRNSSGGDPEIVMHTFNCAGEFFYCNTAELFNSTWYANGTISIGGGNKTNIILPCRIKQFINMWQEVGKAMYAPPISGQIRCSSNITGLLLTRDGGRGNQTDNQTEIFRPVGGDMKNNWRSELYKYKVVRIEPLGIAPTRAKRRVVQREKRAVGIGALFLGFLGAAGSTMGAASMTLTVQARLLLSGIVQQQNNLLRAIEAQQHLLQLTVWGIKQLQARVLAVERYLRDQQLLGIWGCSGKLICTTAVPWNVSWNNRSVDDIWENMTWMQWDREISNYTSLIYTLIEESQNQQEKNEQELLALDKWANLWNWFNITEWLWYIKIFIMIVGGLVGLRIVFAVLSIVNRVRQGYSPLSFQTHLPAQRGPDRPGGIEEEGGESDRDRSGRLVNGFLAIIWIDLRSLCLFSYHHLRDLLLIVTRIVEILGRRGWEILKYWWNLLQYWIQELKNSAVSLLNAIAIAVGEGKDRIIEAFRSIFRAILHIPTRIRQGLERSLL

SEQ ID NO: 55 - DNA, 2610 bp, synthetic construct
  misc_feature 1..2610: note = pGX1055 Env Clade B tier 2 QHO692.42 DNA Sequence
  source 1..2610: mol_type = other DNA
atgcgcgtcaagggaattagaaggaactggcaggggctgtggagatggggaactatgctgctgggaatgctgatgatttgtagggctgccgaaaatctgtgggtgactgtctactatggggtgcctgtctggaaagaggctaccacaactctgttctgcgcatctgatgctaaggcatacgaaacagagaaacacaacgtgtgggccactcatgcttgcgtgccaaccgacccaaatccccaggaagtggtcctgggcaacgtgaccgagaactttaatatgtggaagaacaatatggtggaacagatgcatgaggatatcattagcctgtgggacgagtccctgaagccctgcgtgaaactgacacctctgtgcgtcactctgaattgtaccgatgaagtgaagacatcctacgccaacaaaacttctaatgagacttataagacctctaatgaaaccttcggggagatcaaaaactgtagcttttccgtgccaacaggaattaaggataaagtgcagaacgtctacgccctgttctataagctggacgtgatccccattgacgataacaacaacagctccaagaacaacaacggaagctactctagttacagactgatcaactgcaatacatcagtgattactcaggcttgtcctaaggtcagctttgagcctatcccaattcattactgcgccccagctggcttcgcaatcctgaagtgtaacaacaagaccttcaacggaacaggcccctgcactaacgtgtctaccgtccagtgtacacacggcattagacctgtggtctctacccagctgctgctgaatgggagtctggcagaggaagaggtggtcatcaggagtgaaaacttcactaacaatgccaaaaccatcattgtgcacctgaagaaaagtgtcgagattaactgcacccggccaggcaacaatacaagaaagtcaatccatattggaccaggaagggccttctacgcaaccggggatatcattggagacatccgccaggcccactgtaatctgtcaagcgtgcagtggaacgatacactgaagcagatcgtgatcaagctgggcgagcagttcgggacaaataagactattgcttttaaccagtcctctggcggggaccccgaaatcgtgatgcatagcttcaattgcggaggcgagttcttttactgtaataccacacagctgttcaactccacatgggaatttcacggcaactggacaagatctaacttcaccgagtctaacagtactaccattactctgccttgcaggatcaagcagattgtgaacatgtggcaggaagtcgggaaagctatgtatgcaccccctatcaggggacagattcgctgtagttcaaatatcaccggcctgctgctgacaagagacgggggagtgaacggaacccgagagacattccggcccggcgggggagatatgagagacaactggaggagcgaactgtacaagtataaagtggtcaaaatcgagcctctgggggtggcaccaaccaaggccaaacggagagtggtccagcgcgagaagcgagcagtgggcactattggggccatgttcctgggatttctgggagcagctgggagtaccatgggagcagcctcaatcaccctgacagtgcaggcacgacagctgctgtccggaatcgtgcagcagcagaacaatctgctgcgggccattgaagctcagcagcacatgctgcagctgaccgtgtggggcatcaagcagctgcaggctagggtgctggcagtcgagcggtacctgagagatcagcagctgctgggaatctggggctgcagcgggaagctgatttgtacaactgccgtgccatggaatgcttcatggagcaacaaatcccaggattatatctggaacaatatgacatggatgcagtgggacaaggaaatcaacaactacactaatctgatctactctctgctggaagacagtcagaatcagcaggagaagaacgaacatgagctgctggagctggataaatgggccagcctgtggaactggttcgacatcacccgctggctgtggtacatcaagatcttcatcatgattgtgggcgggctgatcggactgcgaatcgtcattgccgtggtctccattgtgaacagagtcaggcagggatattcccctatctctctgcagacccacttcccagctcctcgcggaccagatcgaccagagggaatcgaagagggaggcggggaccgagatcgagaccggagcctgcgactggtgcacggctccctggccctgatctgggacgatctgaggtcactgtgcatcttcagctaccatagactgagggacctgctgctgatcgtggcccgcgtggtcgaaattctgggaaggcgcggctgggaggctctgaagtactggtggaatctgctgcagtattggtcccaggagctgaaaaacagtgcagtgtcactgctggatgcaactgccatcgctgtggcagaaggcaccgaccggatcattgagatcattcgacgggctttccgcgccatcctgcatattcctacccgcatccgacagggactggagagagcactgctgtgataa

SEQ ID NO: 56 - protein, 868 aa, synthetic construct
  REGION 1..868: note = pGX1055 Env Clade B tier 2 QHO682.42 Amino Acid Sequence
MRVKGIRRNWQGLWRWGTMLLGMLMICRAAENLWVTVYYGVPVWKEATTTLFCASDAKAYETEKHNVWATHACVPTDPNPQEVVLGNVTENFNMWKNNMVEQMHEDIISLWDESLKPCVKLTPLCVTLNCTDEVKTSYANKTSNETYKTSNETFGEIKNCSFSVPTGIKDKVQNVYALFYKLDVIPIDDNNNSSKNNNGSYSSYRLINCNTSVITQACPKVSFEPIPIHYCAPAGFAILKCNNKTFNGTGPCTNVSTVQCTHGIRPVVSTQLLLNGSLAEEEVVIRSENFTNNAKTIIVHLKKSVEINCTRPGNNTRKSIHIGPGRAFYATGDIIGDIRQAHCNLSSVQWNDTLKQIVIKLGEQFGTNKTIAFNQSSGGDPEIVMHSFNCGGEFFYCNTTQLFNSTWEFHGNWTRSNFTESNSTTITLPCRIKQIVNMWQEVGKAMYAPPIRGQIRCSSNITGLLLTRDGGVNGTRETFRPGGGDMRDNWRSELYKYKVVKIEPLGVAPTKAKRRVVQREKRAVGTIGAMFLGFLGAAGSTMGAASITLTVQARQLLSGIVQQQNNLLRAIEAQQHMLQLTVWGIKQLQARVLAVERYLRDQQLLGIWGCSGKLICTTAVPWNASWSNKSQDYIWNNMTWMQWDKEINNYTNLIYSLLEDSQNQQEKNEHELLELDKWASLWNWFDITRWLWYIKIFIMIVGGLIGLRIVIAVVSIVNRVRQGYSPISLQTHFPAPRGPDRPEGIEEGGGDRDRDRSLRLVHGSLALIWDDLRSLCIFSYHRLRDLLLIVARVVEILGRRGWEALKYWWNLLQYWSQELKNSAVSLLDATAIAVAEGTDRIIEIIRRAFRAILHIPTRIRQGLERALL

SEQ ID NO: 57 - DNA, 2562 bp, synthetic construct
  misc_feature 1..2562: note = pGX1034 - Env Clade B tier 2 CAAN5342.A2 DNA Sequence
  source 1..2562: mol_type = other DNA
atgagagtgaaagagattaggaagaactatcggcacctgtggaaatgggggattatgctgctgggaatgctgatgatttgtagcgccacagagaatctgtgggtgactgtctactatggggtgcccgtctggaaggaagccaccacaactctgttctgcgctagcgacgcaaagggatacgagaaagaagtgcacaacgtctgggccacccatgcttgcgtgcctacagatccaaatccccaggaagtggtcctggagaacgtgaccgaaaacttcaacatgtggaaaaacaatatggtggagcagatgcacgaagatatcatttcactgtgggaccagagcctgaagccttgcgtgaaactgactccactgtgcgtcaccctgaattgtagtgacgtgaacaccacatcagtcaatactaccgccagctccatggaaggcggggagatcaagaattgttccttcaacacaactaccagtatgtcagacaagatgcagaaagagtacgctctgttttataccctggatgtggtccccatcgtgaaggaaaacaatacataccggctgatcagttgcaacacatcagtgattactcaggcctgtccaaaagtcagcttcgagcctatcccaattcactattgcgctcccgcaggcttcgctatcctgatgtgcaacaataagacatttgatggcaaagggccttgcaacaacgtgagcaccgtccagtgtacacatggaatcaagccagtggtctcaacccagctgctgctgaatggcagcctggctgaggaagaggtggtcattaggtccgataatttcacagacaacgcaaagactatcattgtgcacctgaacgaatctatcgagattacttgcaccaggcccaacaataacaccagcaaatccatcacaattggacctggacgagccttctacgcaaccggacgaatcattggcgacatccggaaggcacactgtaatattagcggggagaaatggcataacgccctggaacagatcgtgaagaaactgggagaaaagttcgagaatgccacaactatcaggtttaaccagtctagtggaggcgatcaggagattgtgatgcataccttcaactgcgggggagaattcttttactgtaacagcactcagctgtttaattccacctggtggccaaacggcaccacaactgagtggagcaatgaaacctccaacgggacaatcactctgccctgccgcattaagcagatcattaatatgtggcaggaagtgggcaaagctatgtatgcaccccctatctctgggcctattagttgttcaagcaacatcacaggactgctgctggtgcgagatggcgggaatgacaacgagactaatggcaccgaaacattcagaccaggaggcggggatatgcgggacaactggagatccgagctgtacaagtataaagtggtcaagatcgaaccactgggggtggcacccacaaaggccaaacggagagtggtccagagagagaaaagggccgtggggctgggagctatgttcctgggctttctgggagcagctggatctaccatgggagcagccagtatcactctgaccgtgcaggccaggctgctgctgtctgggatcgtccagcagcagaataacctgctgcgcgccattgaggctcagcagcacctgctgcagctgaccgtgtggggcatcaagcagctgcaggctagagtcctggcaattgagaggtacctgaaggaccagcagctgctgggaatctggggatgctccggaaaactgatttgtaccacagccgtgccctggaactcctcttggtctaataagagtctgaaatggatctgggacaatatgacttggatggagtgggaaaaggagattgataattacaccggcatcatctacaacctgctggaagagagtcagaaccagcaggataagaatgaaaaagagctgctggagctggacaagtgggcctcactgtggacttggttcgatatcaccaattggctgtggtacatcaaaatcttcatcatgattgtgggaggcctggtcggactgcggatcgtgttcgcagtcctgtctattgtgaacagggtccgccagggctattcacccctgagctttcagacacgactgccagcacctagggggctggaccgacctgagggaaccgaagaggaagggggagacagagataaggaccgcagtatccgactggtggatggcttcctggctctgatttgggacgatctgagatccctgtgcctgttttcttatcaccgactgcgggacctgctgctgatcgtggcacgggtggtcgagattctgggccatagagggtgggaaatcctgaagtactggtggaacctgctgcagtattggagccaggagctgaaaaattccgccgtgtctctgctgaacgccacagctatcgcagtggccgagggcactgatcgcatcattgaagtgctgcagcgaattggacgagccatcctgcacatccccacccgaattagacagggcctggaaagagcactgctgtgataa

SEQ ID NO: 58 - protein, 852 aa, synthetic construct
  REGION 1..852: note = pGX1034 - Env Clade B tier 2 CAAN5342.A2 Amino Acid Sequence
MRVKEIRKNYRHLWKWGIMLLGMLMICSATENLWVTVYYGVPVWKEATTTLFCASDAKGYEKEVHNVWATHACVPTDPNPQEVVLENVTENFNMWKNNMVEQMHEDIISLWDQSLKPCVKLTPLCVTLNCSDVNTTSVNTTASSMEGGEIKNCSFNTTTSMSDKMQKEYALFYTLDVVPIVKENNTYRLISCNTSVITQACPKVSFEPIPIHYCAPAGFAILMCNNKTFDGKGPCNNVSTVQCTHGIKPVVSTQLLLNGSLAEEEVVIRSDNFTDNAKTIIVHLNESIEITCTRPNNNTSKSITIGPGRAFYATGRIIGDIRKAHCNISGEKWHNALEQIVKKLGEKFENATTIRFNQSSGGDQEIVMHTFNCGGEFFYCNSTQLFNSTWWPNGTTTEWSNETSNGTITLPCRIKQIINMWQEVGKAMYAPPISGPISCSSNITGLLLVRDGGNDNETNGTETFRPGGGDMRDNWRSELYKYKVVKIEPLGVAPTKAKRRVVQREKRAVGLGAMFLGFLGAAGSTMGAASITLTVQARLLLSGIVQQQNNLLRAIEAQQHLLQLTVWGIKQLQARVLAIERYLKDQQLLGIWGCSGKLICTTAVPWNSSWSNKSLKWIWDNMTWMEWEKEIDNYTGIIYNLLEESQNQQDKNEKELLELDKWASLWTWFDITNWLWYIKIFIMIVGGLVGLRIVFAVLSIVNRVRQGYSPLSFQTRLPAPRGLDRPEGTEEEGGDRDKDRSIRLVDGFLALIWDDLRSLCLFSYHRLRDLLLIVARVVEILGHRGWEILKYWWNLLQYWSQELKNSAVSLLNATAIAVAEGTDRIIEVLQRIGRAILHIPTRIRQGLERALL

SEQ ID NO: 59 - DNA, 2580 bp, synthetic construct
  misc_feature 1..2580: note = Env Clade B 6535.3 DNA Sequence
  source 1..2580: mol_type = other DNA
atgaaggtgaaggggacccgcaaaaactaccagagactgtggagatggggcaacatgctgacaatgctgctgggaatgctgatgatttgctccgccacagagaagctgtgggtgactgtctactatggcgtgcctgtctggaaagaagctaccacaactctgttctgcgcatctgaggctaaggcatacgacacagaagtgcacaacgtctgggcaacccatgcctgcgtgccaacagatccaaacccccaggaagtggagctggggaatgtcactgagaacttcaacatgtggaaaaatgacatggtggagcagatgcacgaagacatcattagtctgtgggatcagtcactgaagccttgcgtgcggctgaccccactgtgcgtcacactggactgtactgatctgaacaataccacaaacactaacaatactaccaataccaacagctccaagatcgagggcggggaaatgaagaactgttcattcaacatcacaactaatcgcggagacaagcgacagaaagagtacgccctgctgtataggactgatatcgtgagcattgaaaacacctctagttcataccgcctgatctcatgcaataccagcgtgattacacaggcctgtcctaaggtcacatttgagcctatcccaattcactattgcgccccagctggcttcgctatcctgaagtgtaacgaggataagttcaacggcaccgggccctgcaaaaacgtgtccactgtccagtgtacccatggcattcggcctactgtgagtacccagctgctgctgaatgggtcactggccaaggaggaagtgatcattagatccgccaacctgtctgacaatgctaagatcattatcgtgcagctgaaagatcccgtcgagatcaactgcacacgacctaacaacaacactcggaagagtattaatctgggacccggcagggctttctatgcaacaggagacattatcggcgatatccggcaggcccactgtaacattagcagagctaaatggaatgacactctgagggagatcgctaagaaactggcagaacagttcaataaccgcaccatcgtgtttaaccagagctccggaggcgatcctgagattgtgatgcattctttcaattgcgccggcgaattcttttactgtgacaccagccagctgtttaactccacatggaattcaaacagcacatggaatgatactaataacaataactccaccgagaagattatcctgtcttgccggatcagacagattatcaacaggtggcaggaagtgggcaaggccatgtatgctccccctatcagcgggcccatcaagtgttctagtaatatcacaggactgctgctggctagggacgggggaaatgagactaacgtgacagaaacttttcgcccagcaggaggggacatgcgagataactggagaagcgagctgtacaagtataaagtggtccagatcgaaccactgggcattgcccccacaaaggctaaacggagagtggtccagagagagaagagggcagtggggatgctgggagccatgttcctgggctttctgggggccgctggatcaaccatcggagcagccagcatgaccctgacagtgcaggccaggcagctgctgagcggcatcgtgcagcagcagaataacctgctgcgcgcaattgaggcccagcagcatatgctgcagctgaccgtgtggggcatcaaacagctgcaggcaagagtgctggccgtcgagaggtacctgaaagaccagcagctgctgggcatctgggggtgctctggaaagctgatttgtaccacagccgtgccctggaacacctcctggtctaacaagagtctgaattatatctgggacaacatgacatggatggaatgggagcgggaaattgataattacaccagcctgatctatacactgattgaggaatcccagaaccagcaggagaagaatgagctggaactgctggaactggataaatggggctccctgtggaactggttcagtatctcaaattggctgtggtacatccggatcttcatcatcattgtgggaggcctggtcgggctgagaatcgtgttcaccgtcctgtctattgtgaaccgagtccggcagggatatagcccactgtcctttcagactcgactgccagcaacccagaggggacagccagaccgccctgagggaatcgaggaagaggggggagaaagagacagggcacgctccattcggctggtggatgggttcctggccctgttttgggacgatctgagatctctgtgcctgttcagttaccaccgactgcgggatctgctgctgatcgtggctcgcattgtcgagctgctgggccatcgagggtgggaaatcctgaagtactggtggaacctgctgcagtattggagacaggagctgaagaaatctgcagtgagtctgctgaatactaccgctatcgtggtcgcagagggcaccgaccgcatcattgaagtggtccagcgagcttaccgagcttttctgcatattccccgccgcatccgacagggactggagagagcactgctg

SEQ ID NO: 60 - protein, 860 aa, synthetic construct
  REGION 1..860: note = Env Clade B 6535.3 Amino Acid Sequence
MKVKGTRKNYQRLWRWGNMLTMLLGMLMICSATEKLWVTVYYGVPVWKEATTTLFCASEAKAYDTEVHNVWATHACVPTDPNPQEVELGNVTENFNMWKNDMVEQMHEDIISLWDQSLKPCVRLTPLCVTLDCTDLNNTTNTNNTTNTNSSKIEGGEMKNCSFNITTNRGDKRQKEYALLYRTDIVSIENTSSSYRLISCNTSVITQACPKVTFEPIPIHYCAPAGFAILKCNEDKFNGTGPCKNVSTVQCTHGIRPTVSTQLLLNGSLAKEEVIIRSANLSDNAKIIIVQLKDPVEINCTRPNNNTRKSINLGPGRAFYATGDIIGDIRQAHCNISRAKWNDTLREIAKKLAEQFNNRTIVFNQSSGGDPEIVMHSFNCAGEFFYCDTSQLFNSTWNSNSTWNDTNNNNSTEKIILSCRIRQIINRWQEVGKAMYAPPISGPIKCSSNITGLLLARDGGNETNVTETFRPAGGDMRDNWRSELYKYKVVQIEPLGIAPTKAKRRVVQREKRAVGMLGAMFLGFLGAAGSTIGAASMTLTVQARQLLSGIVQQQNNLLRAIEAQQHMLQLTVWGIKQLQARVLAVERYLKDQQLLGIWGCSGKLICTTAVPWNTSWSNKSLNYIWDNMTWMEWEREIDNYTSLIYTLIEESQNQQEKNELELLELDKWGSLWNWFSISNWLWYIRIFIIIVGGLVGLRIVFTVLSIVNRVRQGYSPLSFQTRLPATQRGQPDRPEGIEEEGGERDRARSIRLVDGFLALFWDDLRSLCLFSYHRLRDLLLIVARIVELLGHRGWEILKYWWNLLQYWRQELKKSAVSLLNTTAIVVAEGTDRIIEVVQRAYRAFLHIPRRIRQGLERALL

SEQ ID NO: 61 - DNA, 2622 bp, synthetic construct
  misc_feature 1..2622: note = Env Clade B THRO.18 DNA Sequence
  source 1..2622: mol_type = other DNA
atgagagtcaaaggaatcaagaagagttttcagcactggtggaaatggggaacaatgctgctgggaatcctgatgatctgtagcgccactgacaagctgtgggtgaccgtctactatggcgtgcctgtctggaaagaagctgtgaccacactgttttgcgcaagcgacgctaaggcatacgatacagaggtgcacaatgtctgggccacacatgcttgcgtgccaactgacccagatccccaggaggtggtcctggaaaacgtgactgagaatttcaacatgtggaagaacaatatggtggaacagatgcacgaggacatcatttcactgtgggatcagagcctgaagccctgcgtgaaactgacacctctgtgcgtcaccctgaattgtacagattataacaatacagccactaacactaccagctccgctacaactaccgcatctagtgccaacaagaccgctaaagaggaagcagtgatgaagaactgttcctttaatatcacaactaacgtgcgggacaaggtcaaaagagaatacgccctgttctataatctggatgtggtcaaactggaggaaggggagacttcttacagactggtgagctgcaacacttccgtggtcacccaggcttgtcccaagatcacctttgagcctatcccaattcactattgcgcccctgctggcttcgcaattctgaagtgtaacaacaagaccttcaacgggactggaccatgcaccaacgtgagtacagtccagtgtactcatggcatcaaacccgtggtctctacccagctgctgctgaatgggagtctggccgagggcggggaagtgatgattcgcagcgcaaacttcactaacaatgccaagaccatcattgtgcagctgtcaaaaagcgtcgccatcaactgcacccggcctaacaataacacatccaagtctattcacatgggcccaggaggcgctttctttgcaaccgggaggatcattggagacatccgcaaagcctactgtaccgtgaatggcacagagtggaacaccacactgaggcagattgtggaaaagttcaagaaacagtttggggagaataagaccatcgtgttcaaaccatcagccgggggagatcccgaaattgtgacacatagctttaactgcggcggggagttcttttactgtaatactaccaacctgttcaattcaagctccacagagctgaatagcacttggtccggaaattctaacgacaccggcaagaacgataccatcacactgccatgccggatcaagcagatcattaatatgtggcagcaagtgggcaaggccatgtatgctccccctatcagcgggaaaattaattgtctgtccaacatcaccggactgctgctgacaagggacggaggctctgatgggggaagtaaaaattctagtaaaaacgaaactggaaccgagatcttccgccctggcgggggagacatgagagataactggaggtccgaactgtacaagtataaagtggtccggatcgagcctctgggagtggcaccaacaaaggctaaacggagagcagtccagcgagagaagcgagacctgggactgggggctctgttcctgggatttctgggagcagctgggagtaccatgggagcagcctcagtgacactgactgtccaggccagacagctgctgtctggcatcgtgcagcagcagaataacctgctgagggcaattgaagcccagcagcacctgctgcagctgaccgtgtggggcatcaagcagctgcaggcacgactgctggctgtggagcggtacctgaaagaccagcagctgctgggaatctggggctgcagcgggaagctgatttgtacaactaccgtgccctggaataacagttggtcaaagaacaaaacatacgagtatatctggaataacatgacttggatcgaatgggagcgcgaaattgataattacacaggcctgatctataacctgattgaaaaaagccagaatcagcaggagaagaacgagaaagaactgctggagctggacaagtgggatagtctgtggtcatggttcagcatcaccaattggctgtggtacatcaagatcttcatcatgattgtgggcgggctgatcgggctgagaatcgtgttcgctgtcctgtccatcgtgaacagggtccgccagggatattcccccctgtctttccagaccaggctgccagcacctcgcgggccagaccgacccgaaggaatcgaggaagagggaggcgagcgagaccgggatagatctggccctctggtgaatgggttcctggccctgatttgggtcgacctgcggtccctgtgcctgttttcttaccataggctgcgcgatctgctgctgatcgtggcacgcattgtcgaactgctgggactgcgaggatgggaggccctgaaatactggtggaacctgctgcagtattggtcccaggagctgaagaatagtgccgtgtcactgctgaacgcaactgccatcgctgtcgcagaaggcaccgatagaatcattgagattctgcagagggtgggacgcgccattctgcatatccccacccgcattcgccagggactggaaagagctctgctg

SEQ ID NO: 62 - protein, 874 aa, synthetic construct
  REGION 1..874: note = Env Clade B THRO.18 Amino Acid Sequence
MRVKGIKKSFQHWWKWGTMLLGILMICSATDKLWVTVYYGVPVWKEAVTTLFCASDAKAYDTEVHNVWATHACVPTDPDPQEVVLENVTENFNMWKNNMVEQMHEDIISLWDQSLKPCVKLTPLCVTLNCTDYNNTATNTTSSATTTASSANKTAKEEAVMKNCSFNITTNVRDKVKREYALFYNLDVVKLEEGETSYRLVSCNTSVVTQACPKITFEPIPIHYCAPAGFAILKCNNKTFNGTGPCTNVSTVQCTHGIKPVVSTQLLLNGSLAEGGEVMIRSANFTNNAKTIIVQLSKSVAINCTRPNNNTSKSIHMGPGGAFFATGRIIGDIRKAYCTVNGTEWNTTLRQIVEKFKKQFGENKTIVFKPSAGGDPEIVTHSFNCGGEFFYCNTTNLFNSSSTELNSTWSGNSNDTGKNDTITLPCRIKQIINMWQQVGKAMYAPPISGKINCLSNITGLLLTRDGGSDGGSKNSSKNETGTEIFRPGGGDMRDNWRSELYKYKVVRIEPLGVAPTKAKRRAVQREKRDLGLGALFLGFLGAAGSTMGAASVTLTVQARQLLSGIVQQQNNLLRAIEAQQHLLQLTVWGIKQLQARLLAVERYLKDQQLLGIWGCSGKLICTTTVPWNNSWSKNKTYEYIWNNMTWIEWEREIDNYTGLIYNLIEKSQNQQEKNEKELLELDKWDSLWSWFSITNWLWYIKIFIMIVGGLIGLRIVFAVLSIVNRVRQGYSPLSFQTRLPAPRGPDRPEGIEEEGGERDRDRSGPLVNGFLALIWVDLRSLCLFSYHRLRDLLLIVARIVELLGLRGWEALKYWWNLLQYWSQELKNSAVSLLNATAIAVAEGTDRIIEILQRVGRAILHIPTRIRQGLERALL